SEQUENCE LISTING (73 sequences)

SEQ ID NO: 1 - protein, 8 aa, mus musculus
GFTFTDNY

SEQ ID NO: 2 - protein, 10 aa, mus musculus
IRNKANGYTT

SEQ ID NO: 3 - protein, 11 aa, mus musculus
ARDVGSNYFDY

SEQ ID NO: 4 - protein, 12 aa, mus musculus
QSLFNSRTRKNY

SEQ ID NO: 5 - protein, 3 aa, mus musculus
WAS

SEQ ID NO: 6 - protein, 8 aa, mus musculus
MQSFNLRT

SEQ ID NO: 7 - protein, 120 aa, artificial sequence
  humanized antibody
EVQLVESGGGLVQPGRSLRLSCTASGFTFTDNYMSWVRQAPGKGLEWVGFIRNKANGYTTEYAASVKGRFTISRDDSKSIAYLQMNSLKTEDTAVYYCARDVGSNYFDYWGQGTLVTVSS

SEQ ID NO: 8 - protein, 120 aa, artificial sequence
  humanized antibody
EVQLVESGGGLVQPGRSLRLSCTASGFTFTDNYMSWVRQAPGKGLEWVGFIRNKANGYTTEYAASVKGRFTISRDNSKSIAYLQMNSLKTEDTAVYYCARDVGSNYFDYWGQGTLVTVSS

SEQ ID NO: 9 - protein, 120 aa, artificial sequence
  humanized antibody
EVQLVESGGGLVQPGRSLRLSCTASGFTFTDNYMSWVRQAPGKGLEWLGFIRNKANGYTTEYAASVKGRFTISRDNSKSIAYLQMNSLKTEDTAVYYCARDVGSNYFDYWGQGTLVTVSS

SEQ ID NO: 10 - protein, 120 aa, artificial sequence
  humanized antibody
EVNLVESGGGLVQPGRSLRLSCTASGFTFTDNYMSWVRQAPGKGLEWLGFIRNKANGYTTDYAASVRGRFTISRDNSKSILYLQMNALRTEDTAVYYCARDVGSNYFDYWGQGTLVTVSS

SEQ ID NO: 11 - protein, 112 aa, artificial sequence
  humanized antibody
DIVMTQSPDSLAVSLGERATINCKSSQSLFNSRTRKNYLAWYQQKPGQPPKLLIYWASTRESGVPDRFSGSGSGTDFTLTISSLQAEDVAVYYCMQSFNLRTFGQGTKVEIK

SEQ ID NO: 12 - protein, 112 aa, artificial sequence
  humanized antibody
DIVMTQSPDSLAVSLGERATINCKSSQSLFNSRTRKNYLAWYQQKPGQPPKLLIYWASARDSGVPDRFSGSGSGTDFTLTISSLQAEDVAVYYCMQSFNLRTFGQGTKVEIK

SEQ ID NO: 13 - protein, 112 aa, artificial sequence
  humanized antibody
DIVMTQSPSSLAVSLGERATMSCKSSQSLFNSRTRKNYLAWYQQKPGQSPKLLIYWASARDSGVPARFTGSGSETYFTLTISRVQAEDLAVYYCMQSFNLRTFGQGTKVEIK

SEQ ID NO: 14 - protein, 112 aa, artificial sequence
  humanized antibody
DIVMTQSPDSLAVSLGERATMSCKSSQSLFNSRTRKNYLAWYQQKPGQPPKLLIYWASARDSGVPDRFSGSGSETYFTLTISRVQAEDLAVYYCMQSFNLRTFGQGTKVEIK

SEQ ID NO: 15 - protein, 112 aa, artificial sequence
  humanized antibody
DIVMTQSPDSLAVSLGERATMSCKSSQSLFNSRTRKNYLAWYQQKPGQPPKLLIYWASARDSGVPDRFTGSGSETYFTLTISRVQAEDVAVYYCMQSFNLRTFGQGTKVEIK

SEQ ID NO: 16 - protein, 112 aa, artificial sequence
  humanized antibody
DIVMTQSPDSLAVSLGERATMSCKSSQSLFNSRTRKNYLAWYQQKPGQPPKLLIYWASARDSGVPDRFTGSGSETYFTLTISSLQAEDLAVYYCMQSFNLRTFGQGTKVEIK

SEQ ID NO: 17 - protein, 112 aa, artificial sequence
  humanized antibody
DIVMTQSPDSLAVSLGERATINCKSSQSLFNSRTRKNYLAWYQQKPGQPPKLLIYWASARDSGVPDRFTGSGSETYFTLTISSLQAEDVAVYYCMQSFNLRTFGQGTKVEIK

SEQ ID NO: 18 - protein, 449 aa, artificial sequence
  humanized antibody
EVQLVESGGGLVQPGRSLRLSCTASGFTFTDNYMSWVRQAPGKGLEWVGFIRNKANGYTTEYAASVKGRFTISRDDSKSIAYLQMNSLKTEDTAVYYCARDVGSNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 449 aa, artificial sequence
  humanized antibody
EVQLVESGGGLVQPGRSLRLSCTASGFTFTDNYMSWVRQAPGKGLEWVGFIRNKANGYTTEYAASVKGRFTISRDNSKSIAYLQMNSLKTEDTAVYYCARDVGSNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 449 aa, artificial sequence
  humanized antibody
EVQLVESGGGLVQPGRSLRLSCTASGFTFTDNYMSWVRQAPGKGLEWLGFIRNKANGYTTEYAASVKGRFTISRDNSKSIAYLQMNSLKTEDTAVYYCARDVGSNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 449 aa, artificial sequence
  humanized antibody
EVNLVESGGGLVQPGRSLRLSCTASGFTFTDNYMSWVRQAPGKGLEWLGFIRNKANGYTTDYAASVRGRFTISRDNSKSILYLQMNALRTEDTAVYYCARDVGSNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 219 aa, artificial sequence
  humanized antibody
DIVMTQSPDSLAVSLGERATINCKSSQSLFNSRTRKNYLAWYQQKPGQPPKLLIYWASTRESGVPDRFSGSGSGTDFTLTISSLQAEDVAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 219 aa, artificial sequence
  humanized antibody
DIVMTQSPDSLAVSLGERATINCKSSQSLFNSRTRKNYLAWYQQKPGQPPKLLIYWASARDSGVPDRFSGSGSGTDFTLTISSLQAEDVAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 219 aa, artificial sequence
  humanized antibody
DIVMTQSPSSLAVSLGERATMSCKSSQSLFNSRTRKNYLAWYQQKPGQSPKLLIYWASARDSGVPARFTGSGSETYFTLTISRVQAEDLAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 219 aa, artificial sequence
  humanized antibody
DIVMTQSPDSLAVSLGERATMSCKSSQSLFNSRTRKNYLAWYQQKPGQPPKLLIYWASARDSGVPDRFSGSGSETYFTLTISRVQAEDLAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 219 aa, artificial sequence
  humanized antibody
DIVMTQSPDSLAVSLGERATMSCKSSQSLFNSRTRKNYLAWYQQKPGQPPKLLIYWASARDSGVPDRFTGSGSETYFTLTISRVQAEDVAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 219 aa, artificial sequence
  humanized antibody
DIVMTQSPDSLAVSLGERATMSCKSSQSLFNSRTRKNYLAWYQQKPGQPPKLLIYWASARDSGVPDRFTGSGSETYFTLTISSLQAEDLAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 219 aa, artificial sequence
  humanizd antibody
DIVMTQSPDSLAVSLGERATINCKSSQSLFNSRTRKNYLAWYQQKPGQPPKLLIYWASARDSGVPDRFTGSGSETYFTLTISSLQAEDVAVYYCMQSFNLRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 24 bp, mus musculus
gggttcaccttcactgataactac

SEQ ID NO: 30 - DNA, 30 bp, mus musculus
attagaaacaaagctaatggttacacaaca

SEQ ID NO: 31 - DNA, 33 bp, mus musculus
gcaagagatgtcggttccaactactttgactac

SEQ ID NO: 32 - DNA, 36 bp, mus musculus
cagagtctgttcaacagtcgaacccgaaagaactac

SEQ ID NO: 33 - DNA, 9 bp, mus musculus
tgggcatcc

SEQ ID NO: 34 - DNA, 24 bp, mus musculus
atgcaatcttttaatcttcggacg

SEQ ID NO: 35 - DNA, 360 bp, artificial sequence
  humanized antibody
gaggtgcagctggtggagtctggcggaggactggtgcagcccggcagaagcctgagactgagctgcaccgccagcggcttcaccttcaccgacaactacatgtcctgggtgcgccaggcccctggaaagggcctggaatgggtgggcttcatccggaacaaggccaacggctacaccacagagtacgccgccagcgtgaagggccggttcaccatcagccgggacgacagcaagagcattgcctacctgcagatgaacagcctgaaaaccgaggacaccgccgtgtactactgcgccagggacgtgggcagcaactacttcgactactggggccagggcacactggtgaccgtgtctagc

SEQ ID NO: 36 - DNA, 360 bp, artificial sequence
  humanized antibody
gaggtgcagctggtggagtctggcggaggactggtgcagcccggcagaagcctgagactgagctgcaccgccagcggcttcaccttcaccgacaactacatgtcctgggtgcgccaggcccctggaaagggcctggaatgggtgggcttcatccggaacaaggccaacggctacaccacagagtacgccgccagcgtgaagggccggttcaccatcagccgggacaacagcaagagcattgcctacctgcagatgaacagcctgaaaaccgaggacaccgccgtgtactactgcgccagggacgtgggcagcaactacttcgactactggggccagggcacactggtgaccgtgtctagc

SEQ ID NO: 37 - DNA, 360 bp, artificial sequence
  humanized antibody
gaggtgcagctggtggagtctggcggaggactggtgcagcccggcagaagcctgagactgagctgcaccgccagcggcttcaccttcaccgacaactacatgtcctgggtgcgccaggcccctggaaagggcctggaatggctgggcttcatccggaacaaggccaacggctacaccacagagtacgccgccagcgtgaagggccggttcaccatcagccgggacaacagcaagagcattgcctacctgcagatgaacagcctgaaaaccgaggacaccgccgtgtactactgcgccagggacgtgggcagcaactacttcgactactggggccagggcacactggtgaccgtgtctagc

SEQ ID NO: 38 - DNA, 360 bp, artificial sequence
  humanized antibody
gaggtgaacctggtggagtctggcggaggactggtgcagcccggcagaagcctgagactgagctgcaccgccagcggcttcaccttcaccgacaactacatgtcctgggtgcgccaggcccctggaaagggcctggaatggctgggcttcatccggaacaaggccaacggctacaccacagactacgccgccagcgtgagaggccggttcaccatcagccgggacaacagcaagagcattctgtacctgcagatgaacgccctgcggaccgaggacaccgccgtgtactactgcgccagggacgtgggcagcaactacttcgactactggggccagggcacactggtgaccgtgtctagc

SEQ ID NO: 39 - DNA, 336 bp, artificial sequence
  humanized antibody
gacatcgtgatgacccagagccccgatagcctggccgtgtctctgggcgagcgggccaccatcaactgcaagagcagccagagcctgttcaacagccggacccggaagaactacctggcctggtatcagcagaagcccggccagccccccaagctgctgatctactgggccagcacaagagaaagcggcgtgcccgaccgcttttctggcagcggcagcggcaccgacttcaccctgaccatcagctccctgcaggccgaggacgtggccgtgtactactgcatgcagagcttcaacctgcggaccttcggccagggcaccaaggtggagatcaag

SEQ ID NO: 40 - DNA, 336 bp, artificial sequence
  humanized antibody
gacatcgtgatgacccagagccccgatagcctggccgtgtctctgggcgagcgggccaccatcaactgcaagagcagccagagcctgttcaacagccggacccggaagaactacctggcctggtatcagcagaagcccggccagccccccaagctgctgatctactgggccagcgccagagacagcggcgtgcccgaccgcttttctggcagcggcagcggcaccgacttcaccctgaccatcagctccctgcaggccgaggacgtggccgtgtactactgcatgcagagcttcaacctgcggaccttcggccagggcaccaaggtggagatcaag

SEQ ID NO: 41 - DNA, 336 bp, artificial sequence
  humanized antibody
gacatcgtgatgacccagagccccagcagcctggccgtgtctctgggcgagcgggccaccatgagctgcaagagcagccagagcctgttcaacagccggacccggaagaactacctggcctggtatcagcagaagcccggccagtcccccaagctgctgatctactgggccagcgccagagatagcggcgtgcccgctcgctttaccggcagcggcagcgagacctacttcaccctgaccatcagccgggtgcaggccgaggacctcgccgtgtactactgcatgcagagcttcaacctgcggaccttcggccagggcaccaaggtggagatcaag

SEQ ID NO: 42 - DNA, 336 bp, artificial sequence
  humanized antibody
gacatcgtgatgacccagtcccccgactccctggccgtgtctctgggcgagcgggccaccatgtcctgcaagtcctcccagtccctgttcaactcccggacccggaagaactacctggcctggtatcagcagaagcccggccagccccccaagctgctgatctactgggcctctgctagagactctggcgtgcccgacagattctccggctccggcagcgagacatacttcaccctgaccatctcccgggtgcaggccgaggatctggccgtgtactactgcatgcagtccttcaacctgcggaccttcggccagggcaccaaggtggaaatcaag

SEQ ID NO: 43 - DNA, 336 bp, artificial sequence
  humanized antibody
gacatcgtgatgacccagtcccccgactccctggccgtgtctctgggcgagcgggccaccatgtcctgcaagtcctcccagtccctgttcaactcccggacccggaagaactacctggcctggtatcagcagaagcccggccagccccccaagctgctgatctactgggcctctgctagagactctggcgtgcccgacagattcaccggctccggcagcgagacatacttcaccctgaccatctcccgggtgcaggccgaggatgtggccgtgtactactgcatgcagtccttcaacctgcggaccttcggccagggcaccaaggtggaaatcaag

SEQ ID NO: 44 - DNA, 336 bp, artificial sequence
  humanized antibody
gacatcgtgatgacccagtcccccgactccctggccgtgtctctgggcgagcgggccaccatgtcctgcaagtcctcccagtccctgttcaactcccggacccggaagaactacctggcctggtatcagcagaagcccggccagccccccaagctgctgatctactgggcctctgctagagactctggcgtgcccgacagattcaccggctccggcagcgagacatacttcaccctgaccatctccagcctgcaggccgaggatctggccgtgtactactgcatgcagtccttcaacctgcggaccttcggccagggcaccaaggtggaaatcaag

SEQ ID NO: 45 - DNA, 336 bp, artificial sequence
  humanized antibody
gacatcgtgatgacccagagccccgatagcctggccgtgtctctgggcgagcgggccaccatcaactgcaagagcagccagagcctgttcaacagccggacccggaagaactacctggcctggtatcagcagaagcccggccagccccccaagctgctgatctactgggccagcgccagagatagcggcgtgcccgaccgctttaccggcagcggcagcgagacctacttcaccctgaccatcagctccctgcaggccgaggacgtggccgtgtactactgcatgcagagcttcaacctgcggaccttcggccagggcaccaaggtggagatcaag

SEQ ID NO: 46 - DNA, 1347 bp, artificial sequence
  humanized antibody
gaggtgcagctggtggagtctggcggaggactggtgcagcccggcagaagcctgagactgagctgcaccgccagcggcttcaccttcaccgacaactacatgtcctgggtgcgccaggcccctggaaagggcctggaatgggtgggcttcatccggaacaaggccaacggctacaccacagagtacgccgccagcgtgaagggccggttcaccatcagccgggacgacagcaagagcattgcctacctgcagatgaacagcctgaaaaccgaggacaccgccgtgtactactgcgccagggacgtgggcagcaactacttcgactactggggccagggcacactggtgaccgtgtctagcgccagcacaaagggcccaagcgtgttcccgctagcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 47 - DNA, 1347 bp, artificial sequence
  humanized antibody
gaggtgcagctggtggagtctggcggaggactggtgcagcccggcagaagcctgagactgagctgcaccgccagcggcttcaccttcaccgacaactacatgtcctgggtgcgccaggcccctggaaagggcctggaatgggtgggcttcatccggaacaaggccaacggctacaccacagagtacgccgccagcgtgaagggccggttcaccatcagccgggacaacagcaagagcattgcctacctgcagatgaacagcctgaaaaccgaggacaccgccgtgtactactgcgccagggacgtgggcagcaactacttcgactactggggccagggcacactggtgaccgtgtctagcgccagcacaaagggcccaagcgtgttcccgctagcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 48 - DNA, 1347 bp, artificial sequence
  humanized antibody
gaggtgcagctggtggagtctggcggaggactggtgcagcccggcagaagcctgagactgagctgcaccgccagcggcttcaccttcaccgacaactacatgtcctgggtgcgccaggcccctggaaagggcctggaatggctgggcttcatccggaacaaggccaacggctacaccacagagtacgccgccagcgtgaagggccggttcaccatcagccgggacaacagcaagagcattgcctacctgcagatgaacagcctgaaaaccgaggacaccgccgtgtactactgcgccagggacgtgggcagcaactacttcgactactggggccagggcacactggtgaccgtgtctagcgccagcacaaagggcccaagcgtgttcccgctagcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 49 - DNA, 1347 bp, artificial sequence
  humanized antibody
gaggtgaacctggtggagtctggcggaggactggtgcagcccggcagaagcctgagactgagctgcaccgccagcggcttcaccttcaccgacaactacatgtcctgggtgcgccaggcccctggaaagggcctggaatggctgggcttcatccggaacaaggccaacggctacaccacagactacgccgccagcgtgagaggccggttcaccatcagccgggacaacagcaagagcattctgtacctgcagatgaacgccctgcggaccgaggacaccgccgtgtactactgcgccagggacgtgggcagcaactacttcgactactggggccagggcacactggtgaccgtgtctagcgccagcacaaagggcccaagcgtgttcccgctagcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 50 - DNA, 657 bp, artificial sequence
  humanized antibody
gacatcgtgatgacccagagccccgatagcctggccgtgtctctgggcgagcgggccaccatcaactgcaagagcagccagagcctgttcaacagccggacccggaagaactacctggcctggtatcagcagaagcccggccagccccccaagctgctgatctactgggccagcacaagagaaagcggcgtgcccgaccgcttttctggcagcggcagcggcaccgacttcaccctgaccatcagctccctgcaggccgaggacgtggccgtgtactactgcatgcagagcttcaacctgcggaccttcggccagggcaccaaggtggagatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 51 - DNA, 657 bp, artificial sequence
  humanized antibody
gacatcgtgatgacccagagccccgatagcctggccgtgtctctgggcgagcgggccaccatcaactgcaagagcagccagagcctgttcaacagccggacccggaagaactacctggcctggtatcagcagaagcccggccagccccccaagctgctgatctactgggccagcgccagagacagcggcgtgcccgaccgcttttctggcagcggcagcggcaccgacttcaccctgaccatcagctccctgcaggccgaggacgtggccgtgtactactgcatgcagagcttcaacctgcggaccttcggccagggcaccaaggtggagatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 52 - DNA, 657 bp, artificial sequence
  humanized antibody
gacatcgtgatgacccagagccccagcagcctggccgtgtctctgggcgagcgggccaccatgagctgcaagagcagccagagcctgttcaacagccggacccggaagaactacctggcctggtatcagcagaagcccggccagtcccccaagctgctgatctactgggccagcgccagagatagcggcgtgcccgctcgctttaccggcagcggcagcgagacctacttcaccctgaccatcagccgggtgcaggccgaggacctcgccgtgtactactgcatgcagagcttcaacctgcggaccttcggccagggcaccaaggtggagatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 53 - DNA, 657 bp, artificial sequence
  humanized antibody
gacatcgtgatgacccagtcccccgactccctggccgtgtctctgggcgagcgggccaccatgtcctgcaagtcctcccagtccctgttcaactcccggacccggaagaactacctggcctggtatcagcagaagcccggccagccccccaagctgctgatctactgggcctctgctagagactctggcgtgcccgacagattctccggctccggcagcgagacatacttcaccctgaccatctcccgggtgcaggccgaggatctggccgtgtactactgcatgcagtccttcaacctgcggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 54 - DNA, 657 bp, artificial sequence
  humanized antibody
gacatcgtgatgacccagtcccccgactccctggccgtgtctctgggcgagcgggccaccatgtcctgcaagtcctcccagtccctgttcaactcccggacccggaagaactacctggcctggtatcagcagaagcccggccagccccccaagctgctgatctactgggcctctgctagagactctggcgtgcccgacagattcaccggctccggcagcgagacatacttcaccctgaccatctcccgggtgcaggccgaggatgtggccgtgtactactgcatgcagtccttcaacctgcggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 55 - DNA, 657 bp, artificial sequence
  humanized antibody
gacatcgtgatgacccagtcccccgactccctggccgtgtctctgggcgagcgggccaccatgtcctgcaagtcctcccagtccctgttcaactcccggacccggaagaactacctggcctggtatcagcagaagcccggccagccccccaagctgctgatctactgggcctctgctagagactctggcgtgcccgacagattcaccggctccggcagcgagacatacttcaccctgaccatctccagcctgcaggccgaggatctggccgtgtactactgcatgcagtccttcaacctgcggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 56 - DNA, 657 bp, artificial sequence
  humanized antibody
gacatcgtgatgacccagagccccgatagcctggccgtgtctctgggcgagcgggccaccatcaactgcaagagcagccagagcctgttcaacagccggacccggaagaactacctggcctggtatcagcagaagcccggccagccccccaagctgctgatctactgggccagcgccagagatagcggcgtgcccgaccgctttaccggcagcggcagcgagacctacttcaccctgaccatcagctccctgcaggccgaggacgtggccgtgtactactgcatgcagagcttcaacctgcggaccttcggccagggcaccaaggtggagatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 57 - DNA, 36 bp, artificial sequence
  optimized codons
cagtccctgttcaactcccggacccggaagaactac

SEQ ID NO: 58 - DNA, 9 bp, artificial sequence
  optimized codons
tgggcctct

SEQ ID NO: 59 - DNA, 24 bp, artificial sequence
  humanized antibody
atgcagtccttcaacctgcggacc

SEQ ID NO: 60 - protein, 110 aa, human
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 61 - protein, 15 aa, human
EPKSCDKTHTCPPCP

SEQ ID NO: 62 - protein, 98 aa, human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 63 - protein, 106 aa, human
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 329 aa, human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - DNA, 330 bp, human
gcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaag

SEQ ID NO: 66 - DNA, 45 bp, human
gagcccaagagctgtgacaagacccacacctgccccccctgccca

SEQ ID NO: 67 - DNA, 294 bp, human
gccagcaccaagggcccaagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtg

SEQ ID NO: 68 - DNA, 318 bp, human
ggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 69 - DNA, 987 bp, human
gccagcaccaagggcccaagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 70 - protein, 469 aa, artificial sequence
  chimeric antibody
MKMWLNWVFLVTLLNGIQCEVNLVESGGGLVQPGGSLRLSCATSGFTFTDNYMSWVRQPPGKALEWLGFIRNKANGYTTDYSASVRGRFTISRDNSQSILYLQMNALRAEDSATYYCARDVGSNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 239 aa, artificial sequence
  chimeric antibody
MESQTLVFISILLWLYGTCGDIVMSQSPSSLAVSAGEKVTMSCKSSQSLFNSRTRKNYLAWYQQKPGQSPKLLIYWASARDSGVPARFTGSGSETYFTLTISRVQAEDLAVYYCMQSFNLRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - DNA, 1404 bp, artificial sequence
  chimeric antibody
atgaagatgtggctgaactgggtgttcctggtgaccctgctgaacggcatccagtgcgaagtgaacctggtggagtctggcggcggactggtgcagcctgggggcagcctgagactgagctgcgccacctccggcttcaccttcaccgacaactacatgagctgggtgcgccagccccctggcaaggccctggaatggctgggcttcatccggaacaaggccaacggctacaccaccgactacagcgccagcgtgcggggcagattcaccatcagccgggacaacagccagagcatcctgtacctgcagatgaacgccctgcgggccgaggacagcgccacctactactgtgcccgggacgtgggcagcaactacttcgactactggggccagggcaccacactgaccgtgtccagcgccagcaccaagggcccctccgtgttcccgctagcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 73 - DNA, 717 bp, artificial sequence
  chimeric antibody
atggagtcacagactctggtcttcatatccatactgctctggttatatggtacctgtggggacattgtgatgtcacagtctccatcctccctggctgtgtcagcaggagagaaggtcactatgagctgcaaatccagtcagagtctgttcaacagtcgaacccgaaagaactacttggcttggtaccagcagaagccagggcagtctcctaaactgctgatctactgggcatccgctagggattctggggtccctgctcgcttcacaggcagtggatctgagacatatttcactctcaccatcagccgtgtgcaggctgaagacctggcagtttattactgcatgcaatcttttaatcttcggacgttcggtggaggcaccaagctggaaatcaaacgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

(19) United States
(12) Patent Application Publication
(10) Pub. No.: US 2014/0120555 A1
(43) Pub. Date: May 1, 2014

(54) ANTI-CXCR4 ANTIBODY WITH EFFECTOR FUNCTIONS AND ITS USE FOR THE TREATMENT OF CANCER

(71) Applicant: PIERRE FABRE MEDICAMENT, Boulogne-Billancourt (FR)

(72) Inventor: Christine Klinguer-Hamour, Groisy (FR)

(21) Appl. No.: 14/126,944

(22) PCT Filed: Jun. 20, 2011
(86) PCT No.: PCT/EP12/61893

§ 371 Date: Dec. 17, 2013

Related U.S. Application Data
(60) Provisional application No. 61/499,004, filed on Jun. 20, 2011.

(30) Foreign Application Priority Data
Jun. 20, 2011 (EP) 11305773.1

(51) Int. Cl. C07K 16/30

(52) U.S. Cl. CPC C07K 16/30
USPC 435/7.23; 530/387.3

(57) ABSTRACT

The present application relates to a method of treating cancer by administering an anti-CXCR4 monoclonal antibody capable of inducing effector function(s).

The present application relates to a method of treating cancer by administering an anti-CXCR4 monoclonal antibody capable of inducing effector function(s).

Chemokines are small, secreted peptides that control the migration of leukocytes along a chemical gradient of ligand, known as chemokine gradient, especially during immune reactions (Zlotnick A. et al., 2000). They are divided into two major subfamilies, CC and CXC, based on the position of their NH₂-terminal cysteine residues, and bind to G protein coupled receptors, whose two major sub families are designated CCR and CXCR. More than 50 human chemokines and 18 chemokine receptors have been discovered so far.

Many cancers have a complex chemokine network that influences the immune-cell infiltration of tumor, as well as tumor cell growth, survival, migration and angiogenesis. Immune cells, endothelial cells and tumor cells themselves express chemokine receptors and can respond to chemokine gradients. Studies of human cancer biopsy samples and mouse cancer models show that cancer cell chemokine-receptor expression is associated with increase metastatic capacity. Malignant cells from different cancer types have different profiles of chemokine-receptor expression, but Chemokine receptor 4 (CXCR4) is most commonly found. Cells from at least 23 different types of human cancers of epithelial, mesenchymal and haematopoietic origin express CXCR4 receptor (Balkwill F. et al., 2004).

Chemokine receptor 4 (also known as fusin, CD184, LESTR or HUMSTR) exists as two isoforms comprising 352 or 360 amino acids. Residue Asn11 is glycosylated, residue Tyr21 is modified by the addition of a sulfate group and Cys 109 and 186 are bond with a disulfide bridge on the extracellular part of the receptor (Juarez J. et al., 2004).

This receptor is expressed by different kind of normal tissues, naïve, non-memory T-cells, regulatory T cells, B-cells, neutrophils, endothelial cells, primary monocytes, dendritic cells, Natural Killer (NK) cells, CD34+ hematopoietic stem cells and at a low level in heart, colon, liver, kidneys and brain. CXCR4 plays a key role in leukocytes trafficking, B cell lymphopoiesis and myelopoiesis.

CXCR4 receptor is over-expressed in a large number of cancers including but not limited to lymphoma, leukemia, multiple myeloma, colon (Ottaiano A. et al., 2004), breast (Kato M. et al., 2003), prostate (Sun Y. X. et al., 2003), lung [small-cell- and non-small-cell-carcinoma (Phillips R. J. et al., 2003)], ovary (Scotton C. J. et al., 2002), pancreas (Koshiba T. et al., 2000), kidneys, brain (Barbero S et al., 2002), glioblastoma and lymphomas.

The unique ligand of CXCR4 receptor described so far is the Stromal-cell-Derived Factor-1 (SDF-1) or CXCL12. SDF-1 is secreted in large amount in lymph nodes, bone marrow, liver, lungs and to a less extent by kidneys, brain and skin. CXCR4 is also recognized by an antagonistic chemokine, the viral macrophage inflammatory protein II (vMIP-II) encoded by human herpesvirus type III.

CXCR4/SDF-1 axis plays a key role in cancer and is implicated directly in migration, invasion leading to metastases. Indeed, cancer cells express CXCR4 receptor, they migrate and enter the systemic circulation. Then cancer cells are arrested in vascular beds in organs that produce high levels of SDF-1 where they proliferate, induce angiogenesis and form metastatic tumors (Murphy P M., 2001). This axis is also involved in cell proliferation via activation of Extracellular-signal-Regulated Kinase (ERK) pathway (Barbero S. et al., 2003) and angiogenesis (Romagnani P., 2004). Indeed, CXCR4 receptor and its ligand SDF-1 clearly promote angiogenesis by stimulating VEGF-A expression which in turns increases expression of CXCR4/SDF-1 (Bachelder R. E. et al., 2002). It is also known that tumor associated macrophages (TAM) accumulated in hypoxic areas of tumors and are stimulated to co-operate with tumor cells and promote angiogenesis. It was observed that hypoxia up-regulated selectively expression of CXCR4 in various cell types including TAM (Mantovani A. et al., 2004). It has been recently demonstrated that CXCR4/SDF-1 axis regulates the trafficking/homing of CXCR4+ hematopoietic stem/progenitor cells (HSC) and could play a role in neovascularization. Evidence indicates that besides HSC, functional CXCR4 is also expressed on stem cells from other tissues (tissue-committed stem cells=TCSCs) so SDF-1 may play a pivotal role in chemottracting CXCR4+TCSCs necessary for organ/tissue regeneration but these TCSC may also be a cellular origin of cancer development (cancer stem cells theory). A stem cell origin of cancer was demonstrated for human leukemia and recently for several solid tumors such as brain and breast. There are several examples of CXCR4+ tumors that may derive from the normal CXCR4+ tissue/organ-specific stem cells such as leukemias, brain tumors, small cell lung cancer, breast cancer, hepatoblastoma, ovarian and cervical cancers (Kucia M. et al., 2005).

Targeting cancer metastases by interfering with CXCR4 receptor was demonstrated in vivo using a monoclonal antibody directed against CXCR4 receptor (Muller A. et al., 2001). Briefly, it was shown that a monoclonal antibody directed against CXCR4 receptor (Mab 173 R&D Systems) decreased significantly the number of lymph node metastases in an orthotopic breast cancer model (MDA-MB231) in SCID mice. Another study (Phillips R. J et al., 2003) also showed the critical role of SDF-1/CXCR4 axis in metastases in an orthotopic lung carcinoma model (A549) using polyclonal antibodies against SDF-1 but in this study there was no effect neither on tumor growth nor on angiogenesis. Several other studies described also the inhibition of either metastasis in vivo using siRNAs duplexes of CXCR4 (Liang Z. et al., 2005) biostable CXCR4 peptide antagonists (Tamamura H. et al., 2003) or tumor growth in vivo using small molecule antagonist of CXCR4 like AMD 3100 (Rubin J. B. et al., 2003; De Falco V. et al., 2007) or Mab (patent WO2004/059285 A2). Thus, CXCR4 is a validated therapeutic target for cancers.

Murine monoclonal antibodies capable of direct interaction with CXCR4, and thus of inhibiting CXCR4 activation, have also been described. Such an inhibition can occur by interfering with: i) the specific binding at cellular membranes of the ligand SDF-1 to the receptor CXCR4, ii) the specific binding at cellular membranes of the GTPyS to the receptor CXCR4, iii) the CXCR4-mediated modulation of cAMP production, and iv) the CXCR4 receptor-mediated mobilization of intracellular calcium stores modulation (see WO 2010/037831).

However, none of these antibodies or siRNA lead to the killing of the CXCR4-expressing cancerous cells. There is therefore still a risk of resumption of the cancer, should the CXCR4-targeted treatment be stopped. There is thus a need for agents which directly target CXCR4-expressing cells, and which are capable of killing said CXCR4-expressing cells.

The present invention relates to a novel property which has never been identified in relation with an antibody targeting CXCR4.

Indeed, the inventors have found that human or humanized antibodies directed against CXCR4 are capable of inducing effector functions against a CXCR4-expressing cell, thus leading to cytotoxic effects against the said cells.

More particularly, the invention relates to a method for the induction of effector function(s) against a CXCR4 expressing cancer cell.

As described in the prior art, treatment of metastatic CXCR4-expressing tumors involved inhibition of migration, invasion, proliferation or angiogenesis, but no direct killing of the CXCR4-expressing cells. In striking contrast, the present invention relates to the induction of cytotoxic effects which lead to the death of the CXCR4-expressing target cell. In particular, the invention provides a human or humanized monoclonal antibody which is capable of inducing one or more effector function(s) against a CXCR4 expressing cancer cell, thus achieving the killing of the said cell. Thus the invention provides a method of treatment of cancer through induction of one or more effector function(s) against a CXCR4 expressing cancer cell by a human or humanized monoclonal antibody.

In a first aspect, the present invention relates to a method of killing a CXCR4-expressing cancer cell with a human or humanized antibody binding to CXCR4, or a CH2-containing binding fragment thereof; said human or humanized antibody comprising a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; wherein said method comprises the step of inducing at least one effector function of the said human or humanized antibody in the presence of effector cells or complement components.

In another aspect, the invention relates to the use of a human or humanized antibody binding to CXCR4, or a CH2-containing binding fragment thereof; wherein said human or humanized antibody comprises a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; for preparing a composition for killing a CXCR4 expressing cancer cell by induction of at least one effector function, in the presence of effector cells or complement components.

In still another aspect, the invention also relates to a human or humanized antibody binding to CXCR4, or a CH2-containing binding fragment thereof; said human or humanized antibody comprising a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; for use in killing a CXCR4 expressing cancer cell by induction of at least one effector function, in the presence of effector cells or complement components.

More specifically, the present invention relates to a method of treating cancer by killing a CXCR4-expressing cancer cell with a human or humanized antibody binding to CXCR4, or a CH2-containing binding fragment thereof; said human or humanized antibody comprising a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; wherein said method comprises the step of inducing at least one effector function of the said human or humanized antibody in the presence of effector cells or complement components.

In another aspect, the invention relates to the use of a human or humanized antibody binding to CXCR4, or a CH2-containing binding fragment thereof; wherein said human or humanized antibody comprises a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; for preparing a composition for treating cancer by killing a CXCR4 expressing cancer cell by induction of at least one effector function, in the presence of effector cells or complement components.

In still another aspect, the invention also relates to a human or humanized antibody binding to CXCR4, or a CH2-containing binding fragment thereof; said human or humanized antibody comprising a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; for use in treating cancer by killing a CXCR4 expressing cancer cell by induction of at least one effector function, in the presence of effector cells or complement components.

The term “antibody” is used herein in the broadest sense and specifically covers monoclonal antibodies (including full length monoclonal antibodies) of any isotype such as IgG, IgM, IgA, IgD, and IgE, polyclonal antibodies, multispecific antibodies, chimeric antibodies, and antibody fragments. An antibody reactive with a specific antigen can be generated by recombinant methods such as selection of libraries of recombinant antibodies in phage or similar vectors, or by immunizing an animal with the antigen or an antigen-encoding nucleic acid.

A “polyclonal antibody” is an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal.

A “monoclonal antibody”, as used herein, is an antibody obtained from a population of substantially homogeneous antibodies, i.e. the antibodies forming this population are essentially identical except for possible naturally occurring mutations which might be present in minor amounts. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.). These antibodies are directed against a single epitope and are therefore highly specific.

An “epitope” is the site on the antigen to which an antibody binds. It can be formed by contiguous residues or by non-contiguous residues brought into close proximity by the folding of an antigenic protein. Epitopes formed by contiguous amino acids are typically retained on exposure to denaturing solvents, whereas epitopes formed by non-contiguous amino acids are typically lost under said exposure.

Preferably, the antibody of the invention is a monoclonal antibody.

A typical antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus (see Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)). The more highly conserved portions of the variable regions are called the “framework regions”.

As used herein, “VH” or “VH” refers to the variable region of an immunoglobulin heavy chain of an antibody, including the heavy chain of an Fv, scFv, dsFv, Fab, Fab′, or F(ab′)2 fragment. Reference to “VL” or “VL” refers to the variable region of the immunoglobulin light chain of an antibody, including the light chain of an Fv, scFv, dsFv, Fab, Fab′, or F(ab′)2 fragment.

Antibody constant domains are not involved directly in binding an antibody to an antigen, but exhibit various effector functions. The heavy chain constant regions that correspond to the different classes of immunoglobulins are called α, δ, ε, γ, and μ, respectively. Depending on the amino acid sequence of the constant region of their heavy chains, antibodies or immunoglobulins can be assigned to different classes, i.e., IgA, IgD, IgE, IgG, and IgM, and several of these may be further divided into subclasses (isotypes), e.g., IgG1, IgG2, IgG3, and IgG4; IgA1 and IgA2 (see, W. E. Paul, ed., 1993, Fundamental Immunology, Raven Press, New York, New York). Papain digestion of antibodies produces two identical antigen binding fragments, called Fab fragments, each with a single antigen binding site, and a residual “Fc” fragment. Although the boundaries of the Fc domain of an immunoglobulin heavy chain might vary, the human IgG heavy chain Fc domain is usually defined to stretch from an amino acid residue at position, according to the EU index, Cys226 or Pro230 in the hinge region, to the carboxyl-terminus thereof containing the CH2 and CH3 domain of the heavy chain (Edelman et al., The covalent structure of an entire gammaG immunoglobulin molecule, PNAS 1969; 63:78-85). For the sake of clarity, it should be stated here that the Cys226/Pro230 residues according to the EU index correspond to the Cys239/Pro243 residues in the Kabat numbering system and to the hinge residues Cys11/Pro15 according to IMGT.

The term “hinge region” is generally defined as stretching from Glu216 to Pro230 of human IgG1 (Burton, Mol Immunol, 22: 161-206, 1985). Hinge regions of other IgG isotypes may be aligned with the IgG1 sequence by placing the first and last cysteine residues forming inter-heavy chain S—S bonds in the same positions. The “CH2 domain” of a human IgG Fc portion (also referred to as “Cγ2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton, Mol Immunol, 22: 161-206, 1985). The “CH3 domain” comprises the stretch of residues C-terminal to a CH2 domain in an Fc portion (i.e., from about amino acid residue 341 to about amino acid residue 447 of an IgG).

IgG immunoglobulins, including monoclonal antibodies have been shown to be N-glycosylated in the constant region of each heavy chain. They contain a single, N-linked glycan at Asn 297 in the CH2 domain on each of its two heavy chains. As used herein, the term “N-glycan” refers to an N-linked oligosaccharide, e.g., one that is attached by an asparagine-N-acetylglucosamine linkage to an asparagine residue of a polypeptide. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine).

N-glycans differ with respect to the number and the nature of branches (antennae) comprising peripheral sugars (e.g., GlcNAc, galactose, fucose, and sialic acid) that are attached to the Man3 core structure. N-glycans are classified according to their branched constituents (e.g., high mannose, complex or hybrid). A “complex, bi-antennary” type N-glycan typically has at least one GlcNAc attached to the 1,3 mannose branch and at least one GlcNAc attached to the 1,6 mannose branch of the trimannose core. Complex bi-antennary N-glycans may also have intrachain substitutions comprising “bisecting” GlcNAc and core fucose (“Fuc”). A “bisecting GlcNAc” is a GlcNAc residue attached to the β-1,4-mannose of the mature core carbohydrate structure.

Complex bi-antennary N-glycans may also have galactose (“Gal”) residues that are optionally modified with sialic acid. Sialic acid addition to the oligosaccharide chain is catalysed by a sialyltransferase, but requires previous attachment of one or more galactose residues by a galactosyltransferase to terminal N-acetylglucosamines. “Sialic acids” according to the invention encompass both 5-N-acetylneuraminic acid (NeuNAc) and 5-glycolylneuraminic acid (NeuNGc).

Oligosaccharides may contain zero (G0), one (G1) or two (G2) galactose residues, as well as one fucose attached to the first GlcNac or not. These forms are noted as G0/G0F, G2/G2F, G1/G1F, respectively (see FIG. 1 of Theillaud, Expert Opin Biol Ther., Suppl 1: S15-S27, 2005). In other words, when both arms of the oligosaccharide chain comprise galactose residues, the maximum moles galactose per mole heavy chain is two and the structure is referred to as G2F when the core is fucosylated and G2 when it is not. When one arm has terminal galactose, the structure is referred to as G1F or G1, depending on whether it is fucosylated or not, while the structure is referred to as G0F or G0, respectively, when there is no terminal galactose.

A secreted IgG immunoglobulin is thus a heterogeneous mixture of glycoforms exhibiting variable addition of the sugar residues fucose, galactose, sialic acid, and bisecting N-acetylglucosamine.

The Fc domains are central in determining the biological functions of the immunoglobulin and these biological functions are termed “effector functions”. These Fc domain-mediated activities are mediated via immunological effector cells, such as killer cells, natural killer cells, and activated macrophages, or various complement components. These effector functions involve activation of receptors on the surface of said effector cells, through the binding of the Fc domain of an antibody to the said receptor or to complement component(s).

The expression “Antibody-dependent cell-mediated cytotoxicity”, “Antibody-dependent cellular cytotoxicity” or “ADCC” refers to a form of cytotoxicity in which Ig bound onto Fc receptors (FcRs) present on certain cytotoxic effector cells enables these cytotoxic effector cells to bind specifically to an antigen-bearing target cell and subsequently kill the target cell with cytotoxins. Lysis of the target cell is extracellular, requires direct cell-to-cell contact, and does not involve complement.

Cell destruction can occur, for example, by lysis or phagocytosis. “Cytotoxic effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs. Cytotoxic effector cells which are capable of cell destruction by lytic means include for, example, natural killer (NK) cells, eosinophils, macrophages and neutrophils.

Of the various human immunoglobulin classes, human IgG1 and IgG3 mediate ADCC more effectively than IgG2 and IgG4.

Advantageously, the human or humanized antibody of the invention, or the CH2-containing fragment thereof, is capable of killing a CXCR4-expressing cancer cell by inducing antibody-dependent cell cytotoxicity (ADCC).

Therefore, in a first preferred embodiment of the method of the invention, the said effector function consists of the antibody-dependent cell cytotoxicity (ADCC).

In other words, the use according the invention is characterized in that said effector function consists of the antibody-dependent cell cytotoxicity (ADCC).

Still in other words, the human or humanized antibody according to the invention is characterized in that said effector function consists of the antibody-dependent cell cytotoxicity (ADCC).

As non limitative examples, the following methods for assessing or quantifying in vitro ADCC can be mentioned: Cytometry using propidium iodide (PI) or calcein, ⁵¹Cr or fluorescent dyes such as calcein-AM, carboxyfluorescein succinimidyl ester (CFSE), 2′,7′-bis-(2carboxyethyl)-5-(and-6)-carboxyfluorescein (BCECF) or Europium, or by measuring the release of cytosolic enzymes such as lactate dehydrogenase (LDH) or ATP. These methods are well-known to the person of skills in the art [see e.g. Jiang et al., “Advances in the assessment and control of the effector functions of therapeutic antibodies”, Nat Rev Drug Discov., 10: 101-110, 2011, and references therein] and need not be further detailed here.

By “complement-dependent cytotoxicity” or “CDC”, it is herein referred a mechanism whereby complement activation triggered by specific antibody binding to an antigen on a cell surface causes the lysis of the target cell, through a series of cascades (complement activation pathways) containing complement-related protein groups in blood. In addition, protein fragments generated by the activation of a complement can induce the migration and activation of immune cells. The first step of complement-dependent cytotoxicity (CDC) activation consists in the binding of C1q protein to at least two Fc domains of the antibody. “C1q” is a polypeptide that includes a binding site for the Fc region of an immunoglobulin. C1q together with two serine proteases, C1r and C1s, forms the complex C1, the first component of the complement-dependent cytotoxicity (CDC) pathway.

In another advantageous embodiment of the invention, the human or humanized antibody, or the CH2-containing fragment thereof, is capable of killing a CXCR4-expressing cancer cell by inducing complement dependent cytotoxicity (CDC).

Therefore, the present invention also relates to a method as described above, wherein the effector function consists of the complement dependent cytotoxicity (CDC).

In other words, the use according to the invention is characterized in that said effector function consists of the complement dependent cytotoxicity (CDC).

Still in other words, the human or humanized antibody according to the invention is characterized in that said effector function consists of the complement dependent cytotoxicity (CDC).

The cytotoxicity of nucleated cells by CDC can be quantitated in vitro by several methods, such as: Trypan blue exclusion, flow cytometry using propidium iodide (PI), ⁵¹Cr release, reduction of tetrazolium salt MTT, redox dye Alamar blue, loss of intracellular ATP, CellTiter-Glo, LDH release or calcein-AM release. These methods are well know to the person of skills in the art [see e.g. Jiang et al., “Advances in the assessment and control of the effector functions of therapeutic antibodies”, Nat Rev Drug Discov., 10: 101-110, 2011, and references therein] and need not be further detailed here.

In a further advantageous embodiment of the invention, both antibody-dependent cell cytotoxicity (ADCC) and the complement dependent cytotoxicity (CDC) are induced, i.e. the human or humanized antibody, or the CH2-containing fragment thereof, is capable of killing a CXCR4-expressing cancer cell by inducing both antibody-dependent cell cytotoxicity (ADCC) and the complement dependent cytotoxicity (CDC).

In a preferred embodiment, the effector functions of the method of the invention consist of the antibody-dependent cell cytotoxicity (ADCC) and the complement dependent cytotoxicity (CDC).

In other words, the use according to the invention is characterized in that said effector functions consist of the antibody-dependent cell cytotoxicity (ADCC) and the complement dependent cytotoxicity (CDC).

Still in other words, the human or humanized antibody according to the invention is characterized in that said effector functions consist of the antibody-dependent cell cytotoxicity (ADCC) and the antibody-dependent cell cytotoxicity (ADCC).

These two effector functions of an antibody are directly associated with the binding of the antibody Fc portion to specific receptors on the surface of immune cells—essentially FcγRIIIa (also referred as FcγRIIIA) and FcγRIIa (also referred as FcγRIIA) expressed on NK cells, macrophages, monocytes for ADCC and the complement cascade protein C1q for CDC.

The precise interactions between the Fc portion of an antibody and FcγRs and C1q have been mapped precisely and the major Fc domain involved in this interaction corresponds to the CH2 domain. The affinity between the Fc portion and the Fc receptors is directly linked to the extent of immune responses that are triggered.

As described above, the human or humanized antibodies directed against CXCR4 are capable of inducing effector functions against a CXCR4-expressing cell, thus leading to cytotoxic effects against the said cells. It is clear that the higher the effector function induction, the greater the cytotoxic effects against the CXCR4-expressing cells. Although some antibodies may display naturally elevated ADCC and/or CDC activity, it may be necessary in other cases to engineer a human or humanized antibody directed against CXCR4 in order to enhance the antibody immune responses and, more particularly, ADCC and/or CDC. Such engineered antibodies are also encompassed by the scope of the present invention.

There are several ways to engineer and to enhance antibody immune responses and, more particularly, ADCC and/or CDC, most of them being based on the direct increase of binding of the Fc portion to the cognate FcγR for ADCC. The major goal is to increase binding to human FcγRa and human FcγRIIa and decrease binding to human FcγRIIb (an inhibitory receptor decreasing immune responses). This can be achieved either by mutating individual amino acid residues within the Fc portion or by modifying the glycan moiety linked to asparagine 297 of the CH2 domain to increase the afucosylated glycan portion. Glycoengineering can be achieved, for example, either by shutting-down (siRNA, KO, etc; Toyohide Shinkawa et al., The absence of fucose but not the presence of galactose or bisecting N-acetylglucosamine of human IgG1 complex-type oligosaccharides shows the critical role of enhancing antibody-dependent cellular cytotoxicity. J. Biol. Chem 2003; 278: 3466-3473) the FUT8 gene in the host cell producing the antibody, or overexpressing a GlcNAc III transferase in the antibody-producing cell (see e.g. Pablo Umana et al. Engineered glycoforms of an antineuroblastoma IgG1 with optimized antibody-dependent cellular cytotoxicity activity. Nat Biotechnol 1999; 17:176-180).

Such antibodies may be obtained by making single or multiple substitutions in the constant domain of the antibody, thus increasing its interaction with the Fc receptors. Methods for designing such mutants can be found for example in Lazar et al. (2006, PNAS, 103(11): 4005-4010) and Okazaki et al. (2004, J. Mol. Biol. 336(5): 1239-49).

It is also possible to use cell lines specifically engineered for production of improved antibodies. In particular, these lines have altered regulation of the glycosylation pathway, resulting in antibodies which are poorly fucosylated or even totally defucosylated. Such cell lines and methods for engineering them are disclosed in e.g. Shinkawa et al. (2003, J. Biol. Chem. 278(5): 3466-3473), Ferrara et al. (Biotechnol. Bioeng. 93(5): 851-61).

Other methods of increasing ADCC have also been described by Li et al. (2006, Nat Biotechnol. 24(2):210-5), Stavenhagen et al. (2008, Advan. Enzyme Regul. 48:152-164), Shields et al. (2001, J. Biol. Chem., 276(9):6591-6604); and WO 2008/006554.

Methods of increasing CDC have been described by Idusogie et al. (2001, J Immunol. 166(4):2571-5), Dall'Acqua et al. (2006, J Immunol, 177(2):1129-38), Michaelsen et al. (1990, Scand J Immunol, 32(5):517-28), Brekke et al. (1993, Mol Immunol, 30(16):1419-25), Tan et al. (1990, Proc; Natl. Acad. Sci. USA, 87:162-166) and Norderhaug et al. (1991, Eur J Immunol, 21(10):2379-84).

A well described technology is the Complement technology developed by Kyowa which consists in making a chimeric human IgG1/IgG3 Fc portion with enhanced CDC (see e.g. WO 2007/011041).

References describing methods of increasing ADCC and CDC include Natsume et al. (2008, Cancer Res. 68(10): 3863-3872). The disclosure of each of these references is included herein by cross reference.

It will be clear for the man skilled in the art that, enhancing ADCC and/or CDC is of a particular interest in the field of the treatment of cancers as it will lead to the killing of the CXCR4-expressing cancerous cells and, as such, will clearly limit the risk of resumption of the cancer, should the CXCR4-targeted treatment be stopped.

By the expression “CH2-containing binding fragment”, it must be understood any fragment or part of an antibody comprising the 6 CDRs of the parental antibody and at least the CH2 domain, which is known as being responsible of inducing an effector function. In a most preferred embodiment, the CH2 must be dimeric, that is to say that it comprises two copies of the CH2.

In another embodiment, the CH2-containing binding fragment comprises the 6 CDRs of the parental antibody and at least the CH2 and the hinge domains.

In another embodiment, the CH2-containing binding fragment comprises the 6 CDRs of the parental antibody and at least the CH1, the hinge and the CH2 domains.

In another embodiment, the CH2-containing binding fragment comprises the 6 CDRs of the parental antibody and at least the CH1 and the CH2 domains.

In another embodiment, the CH2-containing binding fragment comprises the 6 CDRs of the parental antibody and at least the CH1, the CH2 and the CH3 domains.

In still another embodiment, the CH2-containing binding fragment comprises the 6 CDRs of the parental antibody and at least the CH1, the hinge, the CH2 and the CH3 domains, i.e. the full length Fc.

More preferably, the invention comprises the humanized antibodies, their CH2-containing binding fragments, obtained by genetic recombination or chemical synthesis.

According to a preferred embodiment, the human or humanized antibody according to the invention is characterized in that it consists of a monoclonal antibody.

In other words, the method of the invention comprises the use of a human or humanized antibodies, or a CH2-containing binding fragment, which comprises, according to IMGT, a heavy chain comprising the following three CDRs, respectively CDR-H1, CDR-H2 and CDR-H3, wherein:
CDR-H1 comprises the sequence SEQ ID No. 1, or a sequence with at least 80%, preferably 85%, 90%, 95% and 98%, identity after optimal alignment with sequence SEQ ID No. 1;
CDR-H2 comprises the sequence SEQ ID No. 2, or a sequence with at least 80%, preferably 85%, 90%, 95% and 98%, identity after optimal alignment with sequence SEQ ID No. 2; and
CDR-H3 comprises the sequence SEQ ID No. 3, or a sequence with at least 80%, preferably 85%, 90%, 95% and 98%, identity after optimal alignment with sequence SEQ ID No. 3.

Even more preferably, the method of the invention comprises the use of a human or humanized antibodies, or a CH2-containing binding fragment, which comprises, according to IMGT, a light chain comprising the following three CDRs, respectively CDR-L1, CDR-L2 and CDR-L3, wherein:
CDR-L1 comprises the sequence SEQ ID No. 4, or a sequence with at least 80%, preferably 85%, 90%, 95% and 98%, identity after optimal alignment with sequence SEQ ID No. 4;
CDR-L2 comprises the sequence SEQ ID No. 5, or a sequence with at least 80%, preferably 85%, 90%, 95% and 98%, identity after optimal alignment with sequence SEQ ID No. 5; and
CDR-L3 comprises the sequence SEQ ID No. 6, or a sequence with at least 80%, preferably 85%, 90%, 95% and 98%, identity after optimal alignment with sequence SEQ ID No. 6.

In the present description, the terms “polypeptides”, “polypeptide sequences”, “peptides” are interchangeable.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cystein 23 (1st-CYS), tryptophan 41 (CONSERVED-TRP), hydrophobic amino acid 89, cystein 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3Dstructure-DB [Kaas, Q., Ruiz, M. and Lefranc, M.-P., T cell receptor and MHC structural data. Nucl. Acids. Res., 32, D208-D210 (2004)].

In the sense of the present invention, the “percentage identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981) [Ad. App. Math. 2:482], by means of the local homology algorithm of Neddleman and Wunsch (1970) [J. Mol. Biol. 48:443], by means of the similarity search method of Pearson and Lipman (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P).

The percentage identity between two nucleic acid or amino acid sequences is determined by comparing the two optimally-aligned sequences in which the nucleic acid or amino acid sequence to compare can have additions or deletions compared to the reference sequence for optimal alignment between the two sequences. Percentage identity is calculated by determining the number of positions at which the amino acid nucleotide or residue is identical between the two sequences, preferably between the two complete sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/b12.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

For the amino acid sequence exhibiting at least 80%, preferably 85%, 90%, 95% and 98% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below.

Equivalent amino acids can be determined either on their structural homology with the amino acids for which they are substituted or on the results of comparative tests of biological activity between the various antibodies likely to be generated.

As a non-limiting example, table 1 below summarizes the possible substitutions likely to be carried out without resulting in a significant modification of the biological activity of the corresponding modified antibody; inverse substitutions are naturally possible under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

It is known by those skilled in the art that in the current state of the art the greatest variability (length and composition) between the six CDRs is found at the three heavy-chain CDRs and, more particularly, at CDR-H3 of this heavy chain. Consequently, it will be evident that the preferred characteristic CDRs of the antibodies of the invention, or of one of their derived compounds or functional fragments, will be the three CDRs of the heavy chain.

Another embodiment of the invention discloses the use of a human or humanised antibody, or a CH2-containing binding fragment, which comprises:

a heavy chain comprising the following three CDRs:
CDR-H1 of the sequence SEQ ID No. 1 or of a sequence with at least 80%, preferably 85%, 90%, 95% and 98% identity after optimal alignment with sequence SEQ ID No. 1;
CDR-H2 of the sequence SEQ ID No. 2 or of a sequence with at least 80%, preferably 85%, 90%, 95% and 98% identity after optimal alignment with sequence SEQ ID No. 2;
CDR-H3 of the sequence SEQ ID No. 3 or of a sequence with at least 80%, preferably 85%, 90%, 95% and 98% identity after optimal alignment with sequence SEQ ID No 3; and a light chain comprising the following three CDRs:
CDR-L1 of the sequence SEQ ID No. 4 or of a sequence with at least 80%, preferably 85%, 90%, 95% and 98% identity after optimal alignment with sequence SEQ ID No. 4;
CDR-L2 of the sequence SEQ ID No. 5 or of a sequence with at least 80%, preferably 85%, 90%, 95% and 98% identity after optimal alignment with sequence SEQ ID No. 5;
CDR-L3 of the sequence SEQ ID No. 6 or of a sequence with at least 80%, preferably 85%, 90%, 95% and 98% identity after optimal alignment with sequence SEQ ID No. 6.

For more clarity, table 2a below summarizes the various amino acid sequences corresponding to the CDRs of the antibody hz515H7 of the invention; whereas table 2b summarizes the various amino acid sequences corresponding to the variable domains and the full length sequences of the various variants of the humanized antibody of the invention.

TABLE 2a

| Antibody | | | |
|---|---|---|---|
| Hz515H7 | Heavy chain | Light chain | SEQ ID NO. |
| CDR(s) | CDR-H1 | — | 1 |
| | CDR-H2 | — | 2 |
| | CDR-H3 | — | 3 |
| | — | CDR-L1 | 4 |
| | — | CDR-L2 | 5 |
| | — | CDR-L3 | 6 |

TABLE 2b

| Antibody | | | SEQ |
|---|---|---|---|
| Hz515H7 | Heavy chain | Light chain | ID NO. |
| Variable Domains | VH1 | — | 7 |
| | VH1 D76N | — | 8 |
| | VH1 V48L D76N | — | 9 |
| | VH2 | — | 10 |
| | — | VL1 | 11 |
| | — | VL1 T59A E61D | 12 |
| | — | VL2 | 13 |
| | — | VL2.1 | 14 |
| | — | VL2.2 | 15 |
| | — | VL2.3 | 16 |
| | — | VL3 | 17 |
| Complete Sequences | VH1 | — | 18 |
| (without signal peptide) | VH1 D76N | — | 19 |
| | VH1 V48L D76N | — | 20 |
| | VH2 | — | 21 |
| | — | VL1 | 22 |
| | — | VL1 T59A E61D | 23 |
| | — | VL2 | 24 |
| | — | VL2.1 | 25 |
| | — | VL2.2 | 26 |
| | — | VL2.3 | 27 |
| | — | VL3 | 28 |

As an example, for the avoidance of doubt, the expression “VH1” is similar to the expressions “VH Variant 1”, “VH variant 1”, “VH Var 1” or “VH var 1).

It can be mentioned here that the antibody used for the invention was obtained from the humanization of the murine antibody produced by the murine hybridoma filed with the French collection for microorganism cultures (CNCM, Institut Pasteur, Paris, France) on Jun. 25, 2008, under number 1-4019. Said hybridoma was obtained by the fusion of Balb/C immunized mice splenocytes and cells of the myeloma Sp 2/O—Ag 14 lines.

The murine monoclonal antibody, here referred to as 515H7 is secreted by the hybridoma filed with the CNCM on Jun. 25, 2008, under number 1-4019.

In a preferred embodiment, the antibody used in the method of the invention is a humanized antibody.

As used herein, the term “humanized antibody” refers to a chimeric antibody which contain minimal sequence derived from non-human immunoglobulin. A “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass.

In certain embodiments both the variable and constant regions of the antibodies, or antigen-binding fragments, variants, or derivatives thereof are fully human. Fully human antibodies can be made using techniques that are known in the art. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

A “humanized antibody” as used herein refers to an antibody that contains CDR regions derived from an antibody of nonhuman origin, the other parts of the antibody molecule being derived from one (or several) human antibodies. In addition, some of the skeleton segment residues (called FR) can be modified to preserve binding affinity (Jones et al., Nature, 321:522-525, 1986; Verhoeyen et al., Science, 239:1534-1536, 1988; Riechmann et al., Nature, 332:323-327, 1988).

The goal of humanization is a reduction in the immunogenicity of a xenogenic antibody, such as a murine antibody, for introduction into a human, while maintaining the full antigen binding affinity and specificity of the antibody. The humanized antibodies of the invention or fragments of same can be prepared by techniques known to a person skilled in the art (such as, for example, those described in Singer et al., J. Immun., 150:2844-2857, 1992; Mountain et al., Biotechnol. Genet. Eng. Rev., 10:1-142, 1992; and Bebbington et al., Bio/Technology, 10:169-175, 1992). Such humanized antibodies are preferred for their use in methods involving in vitro diagnoses or preventive and/or therapeutic treatment in vivo. Other humanization techniques, also known to a person skilled in the art, such as, for example, the “CDR grafting” technique described by PDL in patents EP 0 451 261, EP 0 682 040, EP 0 939 127, EP 0 566 647 or U.S. Pat. No. 5,530,101, U.S. Pat. No. 6,180,370, U.S. Pat. No. 5,585,089 and U.S. Pat. No. 5,693,761. U.S. Pat. Nos. 5,639,641 or 6,054,297, 5,886,152 and 5,877,293 can also be cited.

By extension, in the case of this present specification, the chimeric antibody c151H7 will be comprised in the expression “humanized antibody”. More particularly, the c515H7 is characterized in that it comprises a heavy chain of sequence SEQ ID No. 70 (corresponding to the nucleotide SEQ ID No. 72) and a light chain of sequence SEQ ID No. 71 (corresponding to the nucleotide SEQ ID No. 73).

The invention relates to the humanized antibodies arising from the murine antibody 515H7 described above, said antibodies being defined by the sequences of their heavy and/or light chains variable domains. Indeed, all the humanized antibodies described herein can be used in the methods of the invention, i.e. they can be used for killing CXCR4-expressing cancer cells by induction of at least one effector function, in the presence of effector cells or complement components, or they can be used for treating cancer by killing CXCR4-expressing cancer cells by induction of at least one effector function, in the presence of effector cells or complement components.

In a preferred embodiment of the methods of the invention, the human or humanized antibody consists of a humanized antibody comprising a heavy chain variable domain selected from the sequences SEQ ID No. 7 to 10 and a light chain variable domain selected from the sequences SEQ ID No. 11 to 17.

In other words, the use according to the invention is characterized in that said human or humanized antibody consists of a humanized antibody comprising a heavy chain variable domain selected from the sequences SEQ ID No. 7 to 10 and a light chain variable domain selected from the sequences SEQ ID No. 11 to 17.

Still in other words, the human or humanized antibody according to the invention is characterized in that it consists of a humanized antibody comprising a heavy chain variable domain selected from the sequences SEQ ID No. 7 to 10 and a light chain variable domain selected from the sequences SEQ ID No. 11 to 17.

A preferred humanized antibody according to the invention consists of a humanized antibody comprising a heavy chain variable domain of sequence SEQ ID No. 8 and a light chain variable domain selected from the sequences SEQ ID No. 11 to 17.

Still a preferred humanized antibody according to the invention consists of a humanized antibody comprising a heavy chain variable domain selected from the sequences SEQ ID No. 7 to 10 and a light chain variable domain of sequence SEQ ID No. 13.

The invention also relates to the humanized antibodies arising from the murine antibody 515H7 described above, said antibodies being defined by the sequences of their full length heavy and/or light chains.

In a preferred embodiment of the method of the invention, the human or humanized antibody consists of a humanized antibody comprising a heavy chain selected from the sequences SEQ ID No. 18 to 21 and a light chain selected from the sequences SEQ ID No. 22 to 28.

In other words, the use according to the invention is characterized in that said human or humanized antibody consists of a humanized antibody comprising a heavy chain selected from the sequences SEQ ID No. 18 to 21 and a light chain selected from the sequences SEQ ID No. 22 to 28.

Still in other words, the human or humanized antibody according to the invention is characterized in that it consists of a humanized antibody comprising a heavy chain selected from the sequences SEQ ID No. 18 to 21 and a light chain selected from the sequences SEQ ID No. 22 to 28.

A preferred humanized antibody according to the invention consists of a humanized antibody comprising a heavy chain of sequence SEQ ID No. 19 and/or a light chain selected from the sequences SEQ ID No. 22 to 28.

Another preferred humanized antibody according to the invention consists of a humanized antibody comprising a heavy chain selected from the sequences SEQ ID No. 18 to 21 and/or a light chain of sequence SEQ ID No. 24.

In a preferred embodiment, the invention relates to the humanized antibody Hz515H7 VH1 D76N VL2, or a derived compound or functional fragment of same, comprising a heavy chain variable region of sequence SEQ ID No. 8, and a light chain variable region of sequence SEQ ID No. 13.

In another preferred embodiment, the invention relates to the humanized antibody Hz515H7 VH1 D76N VL2, or a derived compound or functional fragment of same, comprising a heavy chain of sequence SEQ ID No. 19, and a light chain of sequence SEQ ID No. 24.

In another preferred embodiment, the invention relates to the humanized antibody Hz515H7 VH1 D76N VL2.1, or a derived compound or functional fragment of same, comprising a heavy chain variable region of sequence SEQ ID No. 8, and a light chain variable region of sequence SEQ ID No. 14.

In another preferred embodiment, the invention relates to the humanized antibody Hz515H7 VH1 D76N VL2.1, or a derived compound or functional fragment of same, comprising a heavy chain of sequence SEQ ID No. 19, and a light chain of sequence SEQ ID No. 25.

In another preferred embodiment, the invention relates to the humanized antibody Hz515H7 VH1 D76N VL2.2, or a derived compound or functional fragment of same, comprising a heavy chain variable region of sequence SEQ ID No. 8, and a light chain variable region of sequence SEQ ID No. 15.

In another preferred embodiment, the invention relates to the humanized antibody Hz515H7 VH1 D76N VL2.2, or a derived compound or functional fragment of same, comprising a heavy chain of sequence SEQ ID No. 19, and a light chain of sequence SEQ ID No. 26.

In another preferred embodiment, the invention relates to the humanized antibody Hz515H7 VH1 D76N VL2.3, or a derived compound or functional fragment of same, comprising a heavy chain variable region of sequence SEQ ID No. 8, and a light chain variable region of sequence SEQ ID No. 16.

In another preferred embodiment, the invention relates to the humanized antibody Hz515H7 VH1 D76N VL2.3, or a derived compound or functional fragment of same, comprising a heavy chain of sequence SEQ ID No. 19, and a light chain of sequence SEQ ID No. 27.

In another preferred embodiment, the invention relates to the humanized antibody Hz515H7 VH1 V48L D76N VL1, or a derived compound or functional fragment of same, comprising a heavy chain variable region of sequence SEQ ID No. 9, and a light chain variable region of sequence SEQ ID No. 11.

In another preferred embodiment, the invention relates to the humanized antibody Hz515H7 VH1 V48L D76N VL1, or a derived compound or functional fragment of same, comprising a heavy chain of sequence SEQ ID No. 20, and a light chain of sequence SEQ ID No. 22.

In another preferred embodiment, the invention relates to the humanized antibody Hz515H7 VH1 V48L D76N VL1 T59A E61D, or a derived compound or functional fragment of same, comprising a heavy chain variable region of sequence SEQ ID No. 9, and a light chain variable region of sequence SEQ ID No. 12.

In another preferred embodiment, the invention relates to the humanized antibody Hz515H7 VH1 V48L D76N VL1 T59A E61D, or a derived compound or functional fragment of same, comprising a heavy chain of sequence SEQ ID No. 20, and a light chain of sequence SEQ ID No. 23.

In another preferred embodiment, the invention relates to the humanized antibody Hz515H7 VH1 VL1, or a derived compound or functional fragment of same, comprising a heavy chain variable region of sequence SEQ ID No. 7, and a light chain variable region of sequence SEQ ID No. 11.

In another preferred embodiment, the invention relates to the humanized antibody Hz515H7 VH1 VL1, or a derived compound or functional fragment of same, comprising a heavy chain of sequence SEQ ID No. 18, and a light chain of sequence SEQ ID No. 22.

It must be understood that the above exemplified VH/VL combinations are not limitative. The man skilled in the art could of course, without undue burden and without applying inventive skill, rearrange all the VH and VL disclosed in the present specification.

In a more preferred embodiment of the method of the invention, said human or humanized antibody consists of a humanized antibody comprising a heavy chain variable domain of sequence SEQ ID No. 8 and a light chain variable domain of sequence SEQ ID No. 13.

In other words, the use according to the invention is characterized in that said human or humanized antibody consists of a humanized antibody comprising a heavy chain variable domain of sequence SEQ ID No. 8 and a light chain variable domain of sequence SEQ ID No. 13.

Still in other words, the human or humanized antibody according to the invention is characterized in that it consists of a humanized antibody comprising a heavy chain variable domain of sequence SEQ ID No. 8 and a light chain variable domain of sequence SEQ ID No. 13.

As for the different sequences above described, the preferred antibody (but not exclusive one) will also be described by the sequences of its full length heavy and light chain sequences.

In a more preferred embodiment of the method of the invention, the human or humanized antibody consists of a humanized antibody comprising a heavy chain of sequence SEQ ID No. 19 and a light chain of sequence SEQ ID No. 24.

In other words, the use according to the invention is characterized in that said human or humanized antibody consists of a humanized antibody comprising a heavy chain of sequence SEQ ID No. 19 and a light chain of sequence SEQ ID No. 24.

Still in other words, the human or humanized antibody according to the invention is, characterized in that it consists of a humanized antibody comprising a heavy chain of sequence SEQ ID No. 19 and a light chain of sequence SEQ ID No. 24.

It will be obvious for the man skilled in the art that the antibody of the invention must present structural elements necessary for presenting effector functions. More particularly, the antibody must be of a suitable isotype to allow ADCC and/or CDC. For example, it is known that, of the various human immunoglobulin classes, human IgG1 and IgG3 mediate ADCC more effectively than IgG2 and IgG4. On the other hand, the order of potency for CDC is IgG3≧IgG1>>IgG2≈IgG4 (Niwa et al., J Immunol Methods, 306: 151-160, 2005).

In a preferred embodiment of the method of the invention, the said human or humanized antibody is of the IgG1 isotype.

In other words, the use according to the invention is characterized in that said human or humanized antibody is of the IgG1 isotype.

Still in other words, the human or humanized antibody according to the invention is characterized in that it is of the IgG1 isotype.

In a particular embodiment, the invention relates to a CH2-containing binding fragment of a preferred antibody of the invention consisting of the IgG1 Hz515H7 VH1 D76N VL2.

More particularly, a preferred CH2-containing binding fragment consists of a fragment comprising i) a heavy chain variable domain comprising CDR regions CDR-H1, CDR-H2 and CDR-H3 comprising sequences SEQ ID Nos. 1, 2 and 3, respectively; ii) a light chain variable domain comprising CDR regions CDR-L1, CDR-L2 and CDR-L3 comprising sequences SEQ ID Nos. 4, 5 and 6, respectively; and iii) a CH2 domain comprising at least the sequence SEQ ID No. 60.

In a preferred embodiment of the method of the invention, the CH2-containing binding fragment consists of a fragment comprising the 3 heavy chain CDR-H1, CDR-H2 and CDR-H3 comprising SEQ ID Nos. 1, 2 and 3, respectively; the 3 light chain CDR-L1, CDR-L2 and CDR-L3 comprising SEQ ID Nos. 4, 5 and 6, respectively; and at least the CH2 domain comprising SEQ ID No. 60.

In other words, the use according to the invention is characterized in that said CH2-containing binding fragment consists of a fragment comprising the 3 heavy chain CDR-H1, CDR-H2 and CDR-H3 comprising SEQ ID Nos. 1, 2 and 3, respectively; the 3 light chain CDR-L1, CDR-L2 and CDR-L3 comprising SEQ ID Nos. 4, 5 and 6, respectively; and at least the CH2 domain comprising SEQ ID No. 60.

Still in other words, the human or humanized antibody according to the invention is characterized in that said CH2-containing binding fragment consists of a fragment comprising the 3 heavy chain CDR-H1, CDR-H2 and CDR-H3 comprising SEQ ID Nos. 1, 2 and 3, respectively; the 3 light chain CDR-L1, CDR-L2 and CDR-L3 comprising SEQ ID Nos. 4, 5 and 6, respectively; and at least the CH2 domain comprising SEQ ID No. 60.

Another preferred CH2-containing binding fragment consists of a fragment comprising i) a heavy chain variable domain comprising CDR regions CDR-H1, CDR-H2 and CDR-H3 comprising sequences SEQ ID Nos. 1, 2 and 3, respectively; ii) a light chain variable domain comprising CDR regions CDR-L1, CDR-L2 and CDR-L3 comprising sequences SEQ ID Nos. 4, 5 and 6, respectively; iii) a CH2 domain comprising at least the sequence SEQ ID No. 60; and iv) a hinge domain comprising at least the sequence SEQ ID No. 61.

Still another preferred CH2-containing binding fragment consists of a fragment comprising i) a heavy chain variable domain comprising CDR regions CDR-H1, CDR-H2 and CDR-H3 comprising sequences SEQ ID Nos. 1, 2 and 3, respectively; ii) a light chain variable domain comprising CDR regions CDR-L1, CDR-L2 and CDR-L3 comprising sequences SEQ ID Nos. 4, 5 and 6, respectively; iii) a CH2 domain comprising at least the sequence SEQ ID No. 60; iv) a hinge domain comprising at least the sequence SEQ ID No. 61; and v) a CH1 domain comprising at least the sequence SEQ ID No. 62.

Still another preferred CH2-containing binding fragment consists of a fragment comprising i) a heavy chain variable domain comprising CDR regions CDR-H1, CDR-H2 and CDR-H3 comprising sequences SEQ ID Nos. 1, 2 and 3, respectively; ii) a light chain variable domain comprising CDR regions CDR-L1, CDR-L2 and CDR-L3 comprising sequences SEQ ID Nos. 4, 5 and 6, respectively; iii) a CH2 domain comprising at least the sequence SEQ ID No. 60; iv) a hinge domain comprising at least the sequence SEQ ID No. 61; v) a CH1 domain comprising at least the sequence SEQ ID No. 62; and vi) a CH3 domain comprising at least the sequence SEQ ID No. 63.

In still another preferred embodiment of the invention, a preferred CH2-containing binding fragment consists of a fragment comprising i) a heavy chain variable domain comprising CDR regions CDR-H1, CDR-H2 and CDR-H3 comprising sequences SEQ ID Nos. 1, 2 and 3, respectively; ii) a light chain variable domain comprising CDR regions CDR-L1, CDR-L2 and CDR-L3 comprising sequences SEQ ID Nos. 4, 5 and 6, respectively; and iii) a full length Fc domain comprising at least the sequence SEQ ID No. 64.

For more clarity, the following table 3 illustrates the sequences for each domain for the antibody Hz515H7 VH1 D76N VL2.

TABLE 3

SEQ ID No.

| “domains” | Amino acids | nucleotides |
|---|---|---|
| CH2 | 60 | 65 |
| Hinge | 61 | 66 |
| CH1 | 62 | 67 |
| CH3 | 63 | 68 |
| Full Fc | 64 | 69 |

Based on these elements, it will be clear for the man skilled in the art to generate any CH2-containing binding fragment, derived from any sequence described in the present application, without any undue experiment. As a consequence, any other CH2-containing binding fragment must be considered as part of the scope of the present application.

Table 4a below summarizes the optimized nucleotide sequences corresponding to the CDRs of the antibody hz515H7 of the invention; whereas table 4b summarizes the various optimized nucleotide sequences corresponding to the variable domains and the full length sequences of the various variants of the humanized antibody of the invention.

TABLE 4a

| Antibody | | | |
|---|---|---|---|
| Hz515H7 | Heavy chain | Light chain | SEQ ID NO. |
| optimized | CDR-H1 | — | 29 |
| CDR(s) | CDR-H2 | — | 30 |
| | CDR-H3 | — | 31 |
| | — | CDR-L1 | 32 |
| | | CDR-L1 (bis) | 57 |
| | — | CDR-L2 | 33 |
| | | CDR-L2 (bis) | 58 |
| | — | CDR-L3 | 34 |
| | | CDR-L3 (bis) | 59 |

TABLE 4b

| Antibody | | | |
|---|---|---|---|
| Hz515H7 | Heavy chain | Light chain | SEQ ID NO. |
| Variable | VH1 | — | 35 |
| Domains | VH1 D76N | — | 36 |
| | VH1 V48L D76N | — | 37 |
| | VH2 | — | 38 |
| | — | VL1 | 39 |
| | — | VL1 T59A E61D | 40 |
| | — | VL2 | 41 |
| | — | VL2.1 | 42 |
| | — | VL2.2 | 43 |
| | — | VL2.3 | 44 |
| | — | VL3 | 45 |
| Complete | VH1 | — | 46 |
| Sequences | VH1 D76N | — | 47 |
| (without signal | VH1 V48L D76N | — | 48 |
| peptide) | VH2 | — | 49 |
| | — | VL1 | 50 |
| | — | VL1 T59A E61D | 51 |
| | — | VL2 | 52 |
| | — | VL2.1 | 53 |
| | — | VL2.2 | 54 |
| | — | VL2.3 | 55 |
| | — | VL3 | 56 |

The expression “optimized sequence” means that the codons encoding the amino acids constitutive of the protein of interest (herein the antibody variable domains) have been modified for a better recognition by the translation machinery in a dedicated cell type, herewith mammalian cells. Indeed, it is known to the person of skills in the art that, depending on the source of the gene and of the cell used for expression, a codon optimization may be helpful to increase the expression of the encoded polypeptides of the invention. By “codon optimization”, it is referred to the alterations to the coding sequences for the polypeptides of the invention which improve the sequences for codon usage in the host cell. Codon usage tables are known in the art for mammalian cells, such as e.g. CHO cells, as well as for a variety of other organisms. In addition, optimization can also be achieved by alterations of the polynucleotide sequences which include G/C content adaptation and prevention of stable RNA secondary structure (see as example Kim et al., 1997 Gene 199(1-2):293-301).

The terms “nucleic acid”, “nucleic sequence”, “nucleic acid sequence”, “polynucleotide”, “polynucleotide sequence” and “nucleotide sequence”, used interchangeably in the present description, mean a precise sequence of nucleotides, modified or not, defining a fragment or a region of a nucleic acid, containing unnatural nucleotides or not, and being either a double-strand DNA, a single-strand DNA or transcription products of said DNAs.

The nucleic sequences of the present invention have all been isolated and/or purified, i.e., they were sampled directly or indirectly, for example by a copy, their environment having been at least partially modified. “Nucleic sequences exhibiting a percentage identity of at least 80%, preferably 85%, 90%, 95% and 98%, after optimal alignment with a preferred sequence” means nucleic sequences exhibiting, with respect to the reference nucleic sequence, certain modifications such as, in particular, a deletion, a truncation, an extension, a chimeric fusion and/or a substitution, notably punctual. Preferably, these are sequences which code for the same amino acid sequences as the reference sequence, this being related to the degeneration of the genetic code, or complementarity sequences that are likely to hybridize specifically with the reference sequences, preferably under highly stringent conditions, notably those defined below.

Hybridization under highly stringent conditions means that conditions related to temperature and ionic strength are selected in such a way that they allow hybridization to be maintained between two complementarity DNA fragments. On a purely illustrative basis, the highly stringent conditions of the hybridization step for the purpose of defining the polynucleotide fragments described above are advantageously as follows.

DNA-DNA or DNA-RNA hybridization is carried out in two steps: (1) prehybridization at 42° C. for three hours in phosphate buffer (20 mM, pH 7.5) containing 5×SSC (1×SSC corresponds to a solution of 0.15 M NaCl+0.015 M sodium citrate), 50% formamide, 7% sodium dodecyl sulfate (SDS), 10×Denhardt's, 5% dextran sulfate and 1% salmon sperm DNA; (2) primary hybridization for 20 hours at a temperature depending on the length of the probe (i.e.: 42° C. for a probe>100 nucleotides in length) followed by two 20-minute washings at 20° C. in 2×SSC+2% SDS, one 20-minute washing at 20° C. in 0.1×SSC+0.1% SDS. The last washing is carried out in 0.1×SSC+0.1% SDS for 30 minutes at 60° C. for a probe>100 nucleotides in length. The highly stringent hybridization conditions described above for a polynucleotide of defined size can be adapted by a person skilled in the art for longer or shorter oligonucleotides, according to the procedures described in Sambrook, et al. (Molecular cloning: a laboratory manual, Cold Spring Harbor Laboratory; 3rd edition, 2001).

In order to express the heavy and/or light chain of the human or humanized antibody, or CH2-containing binding fragment thereof, of the invention, the polynucleotides encoding said heavy and/or light chains are inserted into expression vectors such that the genes are operatively linked to transcriptional and translational control sequences.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome.

Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector. However, the invention is intended to include such forms of expression vectors, such as bacterial plasmids, YACs, cosmids, retrovirus, EBV-derived episomes, and all the other vectors that the skilled man will know to be convenient for ensuring the expression of the heavy and/or light chains of the antibodies of the invention. The skilled man will realize that the polynucleotides encoding the heavy and the light chains can be cloned into different vectors or in the same vector. In a preferred embodiment, said polynucleotides are cloned in the same vector.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G4 18 selection). A number of selection systems may be used according to the invention, including but not limited to the Herpes simplex virus thymidine kinase (Wigler et al., Cell 11:223, 1977), hypoxanthine-guanine phosphoribosyltransferase (Szybalska et al., Proc Natl Acad Sci USA 48: 202, 1992), glutamate synthase selection in the presence of methionine sulfoximide (Adv Drug Del Rev, 58: 671, 2006, and website or literature of Lonza Group Ltd.) and adenine phosphoribosyltransferase (Lowy et al., Cell 22: 817, 1980) genes in tk, hgprt or aprt cells, respectively. Also, antimetabolite resistance can be used as the basis of selection for the following genes: dhfr, which confers resistance to methotrexate (Wigler et al., Proc Natl Acad Sci USA 77: 357, 1980); gpt, which confers resistance to mycophenolic acid (Mulligan et al., Proc Natl Acad Sci USA 78: 2072, 1981); neo, which confers resistance to the aminoglycoside, G-418 (Wu et al., Biotherapy 3: 87, 1991); and hygro, which confers resistance to hygromycin (Santerre et al., Gene 30: 147, 1984). Methods known in the art of recombinant DNA technology may be routinely applied to select the desired recombinant clone, and such methods are described, for example, in Ausubel et al., eds., Current Protocols in Molecular Biology, John Wiley & Sons (1993). The expression levels of an antibody can be increased by vector amplification. When a marker in the vector system expressing an antibody is amplifiable, an increase in the level of inhibitor present in the culture will increase the number of copies of the marker gene. Since the amplified region is associated with the gene encoding the IgG antibody of the invention, production of said antibody will also increase (Crouse et al., Mol Cell Biol 3: 257, 1983).

Polynucleotides of the invention and vectors comprising these molecules can be used for the transformation of a suitable mammalian host cell, or any other type of host cell known to the skilled person. The term “recombinant host cell” (or simply “host cell”), as used herein, is intended to refer to a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

Transformation can be by any known method for introducing polynucleotides into a host cell. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei. Preferred mammalian host cells for expressing the recombinant antibodies of the invention include Chinese Hamster Ovary (CHO cells), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Soluble forms of the antibody of the invention can be recovered from the culture supernatant. It may then be purified by any method known in the art for purification of an immunoglobulin molecule, for example, by chromatography (e.g., ion exchange, affinity, particularly by Protein A affinity for Fc, and so on), centrifugation, differential solubility or by any other standard technique for the purification of proteins. Suitable methods of purification will be apparent to a person of ordinary skills in the art.

The present inventors have shown that a human or humanized antibody directed against CXCR4 is capable of killing a CXCR4-expressing cancer cell through induction of at least one effector function of the said antibody.

By “CXCR4-expressing cancer cell”, it is herein referred to a cell of a cancer showing high CXCR4 expression, relative to the CXCR 4 expression level on a normal adult cell. Such cancers include (but are not limited to) the following: carcinomas and adenocarcinomas, including that of the bladder, breast, colon, head-and-neck, prostate, kidney, liver, lung, ovary, pancreas, stomach, cervix, thyroid and skin, and including squamous cell carcinoma; hematopoietic tumors of lymphoid lineage, including multiple myeloma, leukemia, acute and chronic lymphocytic (or lymphoid) leukemia, acute and chronic lymphoblastic leukemia, B-cell lymphoma, T-cell lymphoma, non-Hodgkin lymphoma (e.g. Burkitt's lymphoma); hematopoietic tumors of myeloid lineage, including acute and chronic myelogenous (myeloid or myelocytic) leukemias, and promyelocytic leukemia; tumors of mesenchymal origin, including fibrosarcoma, osteosarcoma and rhabdomyosarcoma; tumors of the central and peripheral nervous system, including astrocytoma, neuroblastoma, glioma, and schwannomas; and other tumors, including melanoma, teratocarcinoma, xeroderma pigmentosum, keratoacanthoma, and seminoma, and other cancers yet to be determined in which CXCR4 is expressed.

In a preferred embodiment of the method of claim the invention, said CXCR4 expressing cancer cell consists of a malignant hematological cell.

In other words, the use according to the invention is characterized in that said CXCR4 expressing cancer cell consists of a malignant hematological cell.

Still in other words, the human or humanized antibody according to the invention is characterized in that said CXCR4 expressing cancer cell consists of a malignant hematological cell.

More particularly, said CXCR4 malignant hematological cell is selected from the group comprising lymphoma cell, leukemia cell or multiple myeloma cell.

In other words, the use according to the invention is characterized in that said CXCR4 malignant hematological cell is selected from the group comprising lymphoma cell, leukemia cell or multiple myeloma cell.

Still in other words, the human or humanized antibody according to the invention is characterized in that said CXCR4 malignant hematological cell is selected from the group comprising lymphoma cell, leukemia cell or multiple myeloma cell.

In anther preferred embodiment, said malignant hematological cell consists of a lymphoma cell.

In other word, the use according to the invention is characterized in that said malignant hematological cell consists of a lymphoma cell.

Still in other words, the human or humanized antibody according to the invention is characterized in that said malignant hematological cell consists of a lymphoma cell.

As above mentioned, effector cells and/or complement components are of particular interest for the invention.

In a more preferred embodiment of the method of the invention, said effector cells comprise NK cells, macrophages, monocytes, neutrophils or eosinophils.

In other words, the use according to the invention is characterized in that said effector cells comprise NK cells, macrophages, monocytes, neutrophils or eosinophils.

Still in other words, the human or humanized antibody according to the invention is characterized in that said effector cells comprise NK cells, macrophages, monocytes, neutrophils or eosinophils.

Based on the following examples, particular interesting properties of the antibodies used in the inventions are described.

In a preferred embodiment of the method of the invention, the induced ADCC level on RAMOS lymphoma cells, after an incubation period of 4 hours, is at least 40%.

In other words, the use according to the invention is characterized in that the induced ADCC level on RAMOS lymphoma cells, after an incubation period of 4 hours, is at least 40%.

Still in other words, the human or humanized antibody according to the invention is characterized in that the induced ADCC level on RAMOS lymphoma cells, after an incubation period of 4 hours, is at least 40%.

In a preferred embodiment of the method of the invention, the induced ADCC level on DAUDI lymphoma cells, after an incubation period of 4 hours, is at least 30%, preferably at least 40%.

In other words, the use according to the invention is characterized in that the induced ADCC level on DAUDI lymphoma cells, after an incubation period of 4 hours, is at least 30%; preferably at least 40%.

Still in other words, the human or humanized antibody according to the invention is characterized in that the induced ADCC level on DAUDI lymphoma cells, after an incubation period of 4 hours, is at least 30%, preferably at least 40%.

In a preferred embodiment of the method of the invention, the induced ADCC level on HeLa cervix cancer cells, after an incubation period of 4 hours, is at least 30%, preferably at least 40%.

In other words, the use according to the invention is characterized in that the induced ADCC level on HaLa cervix cancer cells, after an incubation period of 4 hours, is at least 30%, preferably at least 40%.

Still in other words, the human or humanized antibody according to the invention is characterized in that the induced ADCC level on HeLa cervix cancer cells, after an incubation period of 4 hours, is at least 30%, preferably at least 40%.

Another particular important aspect of the invention relies on the specificity of induced the ADCC and CDC.

In another preferred embodiment of the method of the invention, no significant ADCC is induced on NK cells.

In other words, the use according to the invention is characterized in that no significant ADCC is induced on NK cells.

Still in other words, the human or humanized antibody according to the invention is characterized in that no significant ADCC is induced on NK cells.

The complement components comprise at least the C1q.

In other words, the use according to the invention is characterized in that said complement components comprise at least the C1q.

Still in other words, the human or humanized antibody according to the invention is characterized in that said complement components comprise at least the C1q.

In another preferred embodiment of the method of the invention, the induced CDC level on RAMOS lymphoma cells, after an incubation period of 1 hour, is at least 30%, preferentially at least 50% and most preferably at least 70%.

In other words, the use according to the invention is characterized in that the induced CDC level on RAMOS lymphoma cells, after an incubation period of 1 hour, is at least 30%, preferentially at least 50% and most preferably at least 70%.

Still in other words, the human or humanized antibody according to the invention is characterized in that the induced CDC level on RAMOS lymphoma cells, after an incubation period of 1 hour, is at least 30%, preferentially at least 50% and most preferably at least 70%.

Still another preferred embodiment of the method of the invention, the induced CDC level on NIH3T3 CXCR4 cells, after an incubation period of 1 hour, is at least 30%, preferentially at least 50% and most preferably at least 70%.

In other words, the use according to the invention is characterized in that the induced CDC level on NIH3T3 CXCR4 cells, after an incubation period of 1 hour, is at least 30%, preferentially at least 50% and most preferably at least 70%.

Still in other words, the human or humanized antibody according to the invention is characterized in that the induced CDC level on NIH3T3 CXCR4 cells, after an incubation period of 1 hour, is at least 30%, preferentially at least 50% and most preferably at least 70%.

Still another preferred embodiment of the method of the invention, the induced CDC level on DAUDI lymphoma cells, after an incubation period of 1 hour, is at least 30%, preferentially at least 40%.

In other words, the use according to the invention is characterized in that the induced CDC level on DAUDI lymphoma cells, after an incubation period of 1 hour, is at least 30%, preferentially at least 40%.

Still in other words, the human or humanized antibody according to the invention is characterized in that the induced CDC level on DAUDI lymphoma cells, after an incubation period of 1 hour, is at least 30%, preferentially at least 40%.

In the sense of the ADCC properties of the antibody of the inventions, results have been generated regarding the binding of said antibody with FcγR.

Another particular aspect of the invention relates on that the antibody of the invention, or one of its CH2-containing binding fragment, is capable of binding at least one FcγRs.

In a preferred embodiment of the method of the invention, said at least one FcγRs is FcγRI.

In other words, the use according to the invention is characterized in that said at least one FcγRs is FcγRI. Still in other words, the human or humanized antibody according to the invention is characterized in that said at least one FcγRs is FcγRI.

In another preferred embodiment of the method of the invention, the constant of dissociation (K_{D}) characterizing the binding of the antibody of the invention with the human Fc[gamma]RI, according to the Langmuïr model, is between 1 and 10 nM.

In other words, the use according to the invention is characterized in that the constant of dissociation (KD) characterizing the binding of the antibody of the invention with the human FcγRI, according to the Langmuïr model, is between 1 and 10 nM.

Still in other words, the human or humanized antibody according to the invention is characterized in that the constant of dissociation (KD) characterizing the binding of the antibody of the invention with the human FcγRI, according to the Langmuïr model, is between 1 and 10 nM.

In another preferred embodiment of the method of the invention, said at least one FcγRs is human FcγRIIIa.

In other words, the use according to the invention is characterized in that said at least one FcγRs is human FcγRIIIa.

Still in other words, the human or humanized antibody according to the invention is characterized in that said at least one FcγRs is human FcγRIIIa.

In a more preferred embodiment of the method of the invention, the constant of dissociation (K_{D}) characterizing the binding of the antibody of the invention with the human FcγRIIIa, according to the heterogeneous ligand model, is between 200 and 1000 nM.

In other words, the use according to the invention is characterized in that the constant of dissociation (K_{D}) characterizing the binding of the antibody of the invention with the human FcγRIIIa, according to the heterogeneous ligand model, is between 200 and 1000 nM.

Still in other words, the human or humanized antibody according to the invention is characterized in that the constant of dissociation (K_{D}) characterizing the binding of the antibody of the invention with the human FcγRIIIa, according to the heterogeneous ligand model, is between 200 and 1000 nM.

As used herein, the term “K_{D}” refers to the dissociation constant of a particular antibody/antigen interaction. “Binding affinity” generally refers to the strength of the sum total of non-covalent interactions between a single binding site of a molecule {e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the K_{D}. Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

Preferably, the constant of dissociation is calculated according to the Langmuïr model.

The Langmuïr model is classically described as:

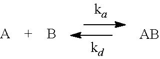

Where A is the analyte, B is the ligand, AB is the non covalent complex between the analyte and the ligand and kₐ and k_{d} are the association and dissociation rates, respectively of this interaction.

In the same way the heterogeneous ligand model where the ligand is considered as a mixture of two components is described by the next two equation systems:

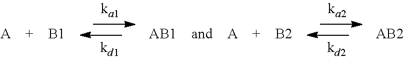

where A is the analyte, B1 is the first component of the ligand, AB1 is the non covalent complex between the analyte and the first component of the ligand, kₐ₁ and k_{d1} are the association and dissociation rates, respectively of this interaction, B2 is the second component of the ligand, AB2 is the non covalent complex between the analyte and the second component of the ligand, kₐ₂ and k_{d2} are the association and dissociation rates respectively, of this interaction.

The BIAevaluation version 3.1 (Biacore AB) has been used for the treatment of BIACORE data.

At last, the invention concerns also a method of treating or preventing a pathological condition associated with the presence of CXCR4 expressing cancer cells comprising the step of administering an effective amount of a human or humanized antibody, or CH2-containing binding fragment thereof; said human or humanized antibody comprising a heavy chain variable domain having the 3 CDRs sequences SEQ ID Nos. 1, 2 and 3 and a light chain variable domain having the 3 CDRs sequences SEQ ID Nos. 4, 5 and 6; wherein at least one effector function of the said human or humanized antibody is induced, in the presence of effector cells or complement components.

In other words, the invention relates to the use of a human or humanized antibody, or a CH2-containing binding fragment thereof, for preparing a composition for the treatment of a pathological condition associated with the presence of CXCR4 expressing cancer cells; wherein said human or humanized antibody comprises a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; wherein at least one effector function of the said human or humanized antibody is induced, in the presence of effector cells or complement components.

Still in other words, the invention relates to a human or humanized antibody specifically recognizing CXCR4, CH2-containing binding fragment, for use in treating a pathological condition associated with the presence of CXCR4 expressing cancer cells; said human or humanized antibody comprising a heavy chain variable domain comprising CDR regions CDR-H1, CDR-H2 and CDR-H3 comprising sequences SEQ ID Nos. 1, 2 and 3, respectively; and a light chain variable domain comprising CDR regions CDR-L1, CDR-L2 and CDR-L3 comprising sequences SEQ ID Nos. 4, 5 and 6, respectively; wherein at least one effector function of the said human or humanized antibody is induced, in the presence of effector cells or complement components.

Preferably, the invention concerns also a method of treating or preventing a pathological condition associated with the presence of CXCR4 expressing cancer cells comprising the step of administering an effective amount of a human or humanized antibody, or CH2-containing binding fragment thereof; said human or humanized antibody comprising a heavy chain variable domain selected from the sequences SEQ ID No. 7 to 10 and a light chain variable domain selected from the sequences SEQ ID No. 11 to 17; wherein at least one effector function of the said human or humanized antibody is induced, in the presence of effector cells or complement components.

In other words, the invention relates to the use of a human or humanized antibody, or a CH2-containing binding fragment thereof, for preparing a composition for the treatment of a pathological condition associated with the presence of CXCR4 expressing cancer cells; said human or humanized antibody comprising a heavy chain variable domain selected from the sequences SEQ ID No. 7 to 10 and a light chain variable domain selected from the sequences SEQ ID No. 11 to 17; wherein at least one effector function of the said human or humanized antibody is induced, in the presence of effector cells or complement components.

Still in other words, the invention relates to a human or humanized antibody specifically recognizing CXCR4, CH2-containing binding fragment, for use in treating a pathological condition associated with the presence of CXCR4 expressing cancer cells; said human or humanized antibody comprising a heavy chain variable domain selected from the sequences SEQ ID No. 7 to 10 and a light chain variable domain selected from the sequences SEQ ID No. 11 to 17; wherein at least one effector function of the said human or humanized antibody is induced, in the presence of effector cells or complement components.

Monoclonal antibodies are known to be N-glycosylated in the constant region of each heavy chain. Specific glycosylation variants have been shown to affect ADCC. For example, lower fucosylation of IgG1s correlates with increased ADCC (Shields et al., J Biol Chem., 277(30): 26733-2640, 2002; Shinkawa et al., J Biol Chem., 278(5): 3466-3473, 2003).

A significant correlation between level of galactose and CDC activity was observed. For example, the CDC activity of rituximab is decreasing with decreasing galactose content (Hodoniczky et al, Biotechnol Prog., 21(6): 1644-1652, 2005)

A representative glycosylation profile of hz515H7 Mab used for ADCC and CDC experiments is shown in the following Table 5.

TABLE 5

| Glycosylation profile (HPLC) | Hz515H7 Mab |
|---|---|
| % G0 or G0FDGlcNac | 5.0 |
| G0F | 82.5 |
| G1F | 9.1 |
| G2F | 0.5 |
| Man5 | 1.8 |

Surprisingly, the Hz515H7 Mab according to the invention induces a high percentage of ADCC and CDC on cells expressing CXCR4, even though around 92% of its carbohydrate chains comprise a fucose residue.

Thus, the present invention also relates to a method of treating cancer by killing CXCR4 expressing cancer cells, comprising the step of administering an effective amount of a human or humanized antibody, or CH2-containing binding fragment thereof; said human or humanized antibody comprising a glycan profile as follows:

| Glycosylation profile (HPLC) | Hz515H7 Mab |
|---|---|
| % G0 or G0FDGlcNac | 5.0 |
| G0F | 82.5 |
| G1F | 9.1 |
| G2F | 0.5 |
| Man5 | 1.8 |

wherein at least one effector function of the said human or humanized antibody is induced, in the presence of effector cells or complement components.

The invention also relates to a humanized antibody binding CXCR4, or a CH2-containing binding fragment thereof, for use in a method of treatment of cancer by killing CXCR4 expressing cancer cells, said human or humanized antibody comprising a glycan profile as follows:

| Glycosylation profile (HPLC) | Hz515H7 Mab |
|---|---|
| % G0 or G0FDGlcNac | 5.0 |
| G0F | 82.5 |
| G1F | 9.1 |
| G2F | 0.5 |
| Man5 | 1.8 |

wherein at least one effector function of the said human or humanized antibody is induced, in the presence of effector cells or complement components.

The invention also relates to the use of a humanized antibody binding CXCR4, or a CH2-containing binding fragment thereof, said human or humanized antibody comprising a glycan profile as follows:

| Glycosylation profile (HPLC) | Hz515H7 Mab |
|---|---|
| % G0 or G0FDGlcNac | 5.0 |
| G0F | 82.5 |
| G1F | 9.1 |
| G2F | 0.5 |
| Man5 | 1.8 |

for preparing a medicament for treating cancer by killing CXCR4 expressing cancer cells, wherein at least one effector function of the said human or humanized antibody is induced, in the presence of effector cells or complement components.

As shown in the Examples herein, the human or humanized antibody, or CH2-containing binding fragment thereof, of the present invention have anti-tumoral activity, at least through induction of ADCC and/or CDC responses, and are thus useful in the treatment of metastatic tumors and diseases such as cancer.

The terms “treating” or “treatment” refer to administering or the administration of a composition described herein in an amount, manner, and/or mode effective to improve a condition, symptom, or parameter associated with a disorder or to prevent progression or exacerbation of the disorder (including secondary damage caused by the disorder) to either a statistically significant degree or to a degree detectable to one skilled in the art.

Another aspect of the invention relates to pharmaceutical compositions of the human or humanized antibody, or CH2-containing binding fragment thereof.

The pharmaceutical composition of the invention may contain, in addition to the carrier and the human or humanized antibody, or CH2-containing binding fragment thereof, various diluents, fillers, salts, buffers, stabilizers, solubilizers, and other materials well known in the art.

As used herein, “pharmaceutically acceptable carrier” includes any and all solvents, buffers, salt solutions, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of media and agents for pharmaceutically active substances is well known in the art. As detailed herebelow, additional active compounds can also be incorporated into the compositions, such as anti-cancer and/or anti-angiogenesis agents; in particular, the additional active compound can be an anti-angiogenic agent, a chemotherapeutic agent, or a low-molecular weight agent. A typical pharmaceutical composition for intravenous infusion could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17th ed., Mack Publishing Company, Easton, Pa. (1985), and the 18^{th} and 19^{th} editions thereof, which are incorporated herein by reference.

The human or humanized antibody, or CH2-containing binding fragment thereof in the composition preferably is formulated in an effective amount. An “effective amount” refers to an amount effective, at dosages and for periods of time necessary, to achieve the desired result, such as induction of apoptosis in tumor cells. A “therapeutically effective amount” means an amount sufficient to influence the therapeutic course of a particular disease state. A therapeutically effective amount is also one in which any toxic or detrimental effects of the agent are outweighed by the therapeutically beneficial effects.

For therapeutic applications, the human or humanized antibody, or CH2-containing binding fragment thereof, of the invention is administered to a mammal, preferably a human, in a pharmaceutically acceptable dosage form such as those discussed above, including those that may be administered to a human intravenously as a bolus or by continuous infusion over a period of time, by intramuscular, intraperitoneal, intracerebrospinal, subcutaneous, intraarticular, intrasynovial, intrathecal, oral, topical, or inhalation routes. The said human or humanized antibody, or CH2-containing binding fragment thereof, is also suitably administered by intratumoral, peritumoral, intralesional, or perilesional routes, to exert local as well as systemic therapeutic effects. The intraperitoneal route is expected to be particularly useful, for example, in the treatment of ovarian tumors.

Dosage regimens may be adjusted to provide the optimum response. For example, a single bolus may be administered, several divided doses may be administered over time, or the dose may be proportionally reduced or increased. The compositions of the invention can be administered to a subject to effect cell growth activity in a subject. As used herein, the term “subject” is intended to include living organisms in which apoptosis can be induced, and specifically includes mammals, such as rabbits, dogs, cats, mice, rats, monkey transgenic species thereof, and preferably humans.

The human or humanized antibody of the invention, or CH2-containing binding fragment thereof, and the pharmaceutical compositions of the invention are especially useful in the treatment or prevention of several types of cancers including (but not limited to) the following: carcinomas and adenocarcinomas, including that of the bladder, breast, colon, head-and-neck, prostate, kidney, liver, lung, ovary, pancreas, stomach, cervix, thyroid and skin, and including squamous cell carcinoma; hematopoietic tumors of lymphoid lineage, including multiple myeloma, leukemia, acute and chronic lymphocytic (or lymphoid) leukemia, acute and chronic lymphoblastic leukemia, B-cell lymphoma, T-cell lymphoma, non-Hodgkin lymphoma (e.g. Burkitt's lymphoma); hematopoietic tumors of myeloid lineage, including acute and chronic myelogenous (myeloid or myelocytic) leukemias, and promyelocytic leukemia; tumors of mesenchymal origin, including fibrosarcoma, osteosarcoma and rhabdomyosarcoma; tumors of the central and peripheral nervous system, including astrocytoma, neuroblastoma, glioma, and schwannomas; and other tumors, including melanoma, teratocarcinoma, xeroderma pigmentosum, keratoacanthoma, and seminoma, and other cancers yet to be determined in which CXCR4 is expressed. By cancers having CXCR4 expression, it is herein referred to cancers displaying high CXCR4 expression, relative to the CXCR 4 expression level on a normal adult cell.

The human or humanized antibody of the invention, or CH2-containing binding fragment thereof, and the pharmaceutical compositions of the invention are mainly useful for treating leukemia, lymphoma and cancers resistant to the commonly used anticancer agents as the anti-CXCR4 antibodies of the invention have a unique mechanism of action.

In a preferred embodiment of the method of the invention, said pathological condition associated with the presence of CXCR4 expressing cancer cells consists of lymphoma, leukemia or multiple myeloma, preferentially lymphoma.

In other words, the use according to the invention is characterized in that said pathological condition associated with the presence of CXCR4 expressing cancer cells consists of lymphoma, leukemia or multiple myeloma, preferentially lymphoma.

Still in other words, the human or humanized antibody according to the invention is characterized in that said pathological condition associated with the presence of CXCR4 expressing cancer cells consists of lymphoma, leukemia or multiple myeloma, preferentially lymphoma.

As a non limitative example, a process of detecting in vitro the presence and/or the location of a CXCR4 expressing tumor in a subject, comprises the steps of:

(a) contacting a sample from the subject with a humanized antibody heavy chain and/or a humanized antibody light chain and/or a humanized antibody, or a derived compound or functional fragment of same, according capable of binding specifically with CXCR4; and
(b) detecting the binding of said antibody with the sample.

Particularly, a process of determining in vitro or ex vivo the expression level of CXCR4 in a CXCR4 expressing tumor from a subject comprises the steps of:

(a′) contacting a sample from the subject with a humanized antibody heavy chain and/or a humanized antibody light chain and/or a humanized antibody, or a derived compound or functional fragment of same, capable of binding specifically to CXCR4; and
(b′) quantifying the level of antibody binding to CXCR4 in said sample.

In a preferred embodiment, the CXCR4 expression level can be measured by immunohistochemistry (IHC) or FACS analysis.

As used herein, the term “an oncogenic disorder associated with expression of CXCR4” or “CXCR4-expressing cancer cell” is intended to include diseases and other disorders in which the presence of high levels or abnormally low levels of CXCR4 (aberrant) in a subject suffering from the disorder has been shown to be or is suspected of being either responsible for the pathophysiology of the disorder or a factor that contributes to a worsening of the disorder. Alternatively, such disorders may be evidenced, for example, by an increase in the levels of CXCR4 on the cell surface in the affected cells or tissues of a subject suffering from the disorder. The increase in CXCR4 levels may be detected, for example, using the antibody 515H7 or hz515H7 of the invention. More, it refers to cells which exhibit relatively autonomous growth, so that they exhibit an aberrant growth phenotype characterized by a significant loss of control of cell proliferation. Alternatively, the cells may express normal levels of CXCR4 but are marked by abnormal proliferation.

The invention also describes a method for the screening of humanized antibodies binding CXCR4, or CH2-containing binding fragments thereof, for use in killing a CXCR4 expressing cancer cell by induction of at least one effector function, in the presence of effector cells or complement components, wherein said method comprises at least one selection step selected from:
selecting antibodies inducing an ADCC level on RAMOS lymphoma cells, after an incubation period of 4 hours, of at least 40%;
selecting antibodies inducing a CDC level on RAMOS lymphoma cells, after an incubation period of 1 hour, of at least 30%, preferentially of at least 50% and most preferably of at least 70%;
selecting antibodies inducing a CDC level on NIH3T3 CXCR4 cells, after an incubation period of 1 hour, of at least 30%, preferentially of at least 50% and most preferably of at least 70%;
selecting antibodies binding FcγRI with a constant of dissociation (KD), according to the Langmuïr model, between 1 and 10 nM;
selecting antibodies binding FcγRIIIA with a constant of dissociation (KD), according to the heterogeneous ligand model, between 200 and 1000 nM.

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See Ausubel et al., Current Protocols in Molecular Biology, Eds., John Wiley & Sons, Inc. New York, 1995; Remington's Pharmaceutical Sciences, 17th ed., Mack Publishing Co., Easton, Pa., 1985; and Sambrook et al., Molecular cloning: A laboratory manual 2nd edition, Cold Spring Harbor Laboratory Press—Cold Spring Harbor, N.Y., USA, 1989; Introduction to Glycobiology, Maureen E. Taylor, Kurt Drickamer, Oxford Univ. Press (2003); Worthington Enzyme Manual, Worthington Biochemical Corp. Freehold, N.J.; Handbook of Biochemistry: Section A Proteins, Vol I 1976 CRC Press; Handbook of Biochemistry: Section A Proteins, Vol II 1976 CRC Press; Essentials of Glycobiology, Cold Spring Harbor Laboratory Press (1999)). The nomenclatures used in connection with, and the laboratory procedures and techniques of, molecular and cellular biology, protein biochemistry, enzymology and medicinal and pharmaceutical chemistry described herein are those well known and commonly used in the art.

Unless defined otherwise, all technical and scientific terms used herein have the same meaning as is commonly understood by one of the skill in the art to which this invention belongs.

Other characteristics and advantages of the invention appear further in the description with the examples and figures whose legends are presented below.

### FIGURE LEGENDS

FIG. 1 shows the amino acid sequences alignment of 515H7 heavy chain variable domain with the human germline IGHV3-49*04 and IGHJ4*01. The 515H7 VH amino acid sequence is aligned with the selected human acceptor framework sequences. VH1 and VH2 (VH3 is not represented) sequences correspond to implemented humanized variants of the 515H7 VH domain, with back mutated residues in bold. Variant 1 VH1 carries no back mutated residue and represents a fully human variant. Variant VH2 has 8 back mutations and is the most murine variant. Variant VH3 carries 5 back mutations (not represented).

FIG. 2 shows the amino acid sequences alignment of 515H7 light chain with the human germline IGKV4-1*01 and IGKJ1*01. The 515H7 VL amino acid sequence is aligned with the selected human acceptor framework sequences. VL1 to VL3 sequences correspond to implemented humanized variants of the 515H7 VL domain, with back mutated residues in bold. Variant VL1 carries no back mutated residue and represents the most human variant. Variant VL2 has 13 back mutations and is the most murine variant. Variant VL3 carries 5 back mutations.

FIGS. 3A-3F show cross blocking of the biotinylated murine antibody 515H7 by the chimeric 515H7 and different variants of the humanized 515H7. The activity of the humanized variants of 515H7 (hz515H7) to cross block the parental murine antibody 515H7 was evaluated by flow cytometry using CXCR4 transfected NIH3T3 cells. The activity of the humanized variants was compared to the chimeric 515H7. The cross blocking activity of the three different variants of VH (VH1-VH3) combined with the chimeric VL (cVL) were very similar (FIG. 3A-FIG. 3C). No reduction in the activity of VH variant 1 (VH1, the variant with no back mutations) was determined when combined with variant 1 and 2 of VL. A significant reduction of the activity was detected for the construct hz515H7 VH1 VL3 (FIGS. 3D-3F).

FIG. 4 shows the BRET assay for testing the activity of the humanized antibody 515H7 variant VH1 VL1. The activity of the humanized variant 515H7 VH variant 1 VL variant 1 (hz515H7 VH1 VL1) was evaluated by its capacity to inhibit SDF-1 mediated signal transduction. This variant showed only a minor inhibition of the SDF-1 mediated signal transduction as determined by BRET. SDF-1 was used at a concentration of 100 nM.

FIGS. 5A-5D show comparisons of different mutants of the VH1 with single or double back mutations and combinations of different VL variants with hz515H7 VH1D76N. Single and double back mutations were made in the VH1 and combined with the VL1. These constructs were evaluated in BRET assays (FIGS. 5A-5C). Of these single back mutants only the construct with the back mutation D76N showed an increased inhibition of the SDF-1 mediated signal transduction. None of the double back mutant in VH had strong inhibitory activity (FIG. 5C). The single back mutant D76N of the VH1 was combined with different variants of VL. The SDF-1 concentration was 100 nM.

FIG. 6 shows ranking of different mutants of the VH1 and VL1 with single or double back mutations in comparison to the construct VH1 D76N VL2. Single and double back mutations were made in the VH1 and combined with the VL1. All constructs were evaluated in BRET assays and their percent inhibition calculated. The SDF-1 concentration was 100 nM.

FIGS. 7A-7B show inhibition of SDF-1 binding by different constructs of the humanized 515H7 and correlation between result obtained by FACS and BRET. The different variants of the humanized antibody 515H7 with a strong activity in blocking the recruitment of β-arrestin were tested in their capacity to inhibit the binding of biotinylated SDF-1 in flow cytometry (FACS) (A). These were compared with VH1 and VL1. Results from the FACS-based assay are correlated with the results obtained by BRET (B).

FIG. 8 shows the amino acid sequences alignment of hz515H7 VL2 and further humanized versions 515H7 VL2.1, 515H7 VL2.2 and 515H7 VL2.3. The 515H7 VL amino acid sequence is aligned with the selected human acceptor framework sequences. VL2.1, VL2.2 and VL2.3 sequences correspond to implemented humanized variants of the humanized 515H7 VL2, with mutated residues in bold. VL2.1 and VL2.2 carry 4 more humanized residues whereas VL2.3 contains 5 more human residues.

FIGS. 9A-9C show the 515H7 humanized Mabs (hz515H7 VH1 D76N VL2, hz515H7 VH1 D76N VL2.1, hz515H7 VH1 D76N VL2.2 and hz515H7 VH1 D76N VL2.3) specific binding to CXCR4 on NIH3T3-CXCR4 (FIG. 9A) U937 (FIG. 9B) and Ramos cells (FIG. 9C).

FIG. 10 show antibody dependent cellular cytotoxicity (ADCC) effect of hz515H7VH1D76NVL2 Mab on cells expressing CXCR4, Ramos cells (FIG. 10A) and Natural killer cells (NK) (FIG. 10B)

FIG. 11 show antibody dependent cellular cytotoxicity (ADCC) effect of c515H7 Mab on cells expressing CXCR4, Ramos cells (FIG. 11A) and Natural killer cells (NK) (FIG. 11B)

FIG. 12 shows complement dependent cytotoxicity (CDC) effect of hz515H7VH1D76NVL2 Mab on NIH3T3-CXCR4 cell line and Ramos cells expressing CXCR4

FIG. 13 shows complement dependent cytotoxicity (CDC) effect of c515H7 Mab on Ramos cells expressing CXCR4

FIG. 14 show complement dependent cytotoxicity (CDC) dose effect of hz515H7VH1D76NVL2 (FIG. 14A) and c515H7 (FIG. 14B) Mabs on Ramos cells expressing CXCR4

FIG. 15: Binding of the recombinant human FcγRI with hz515H7VH1D76NVL2 Mab immobilized on a CM4 sensorchip. 6 different concentrations of h-FcγRI were tested (200, 100, 50, 25, 12.5 and 6.25 nM).

FIG. 16: Binding of the recombinant human FcγRIIIA with hz515H7VH1D76NVL2 Mab immobilized on a CM4 sensorchip. 5 different concentrations of h-FcγRIIIA were tested (1000, 500, 250, 125 and 62.5 nM).

FIG. 17: Constant of Dissociation determination of the h-FcγRIIIA/hz515H7VH1D76NVL2 complex by steady-state analysis using the response at the end of the association phase versus the human FcγRIIIA concentrations (1000, 500, 250, 125 and 62.5 nM) plot.

FIG. 18: Binding of the recombinant mouse FcγRI with hz515H7VH1D76NVL2 Mab immobilized on a CM4 sensorchip. 5 different concentrations of m-FcγRI were tested (400, 200, 100, 50 and 25 nM).

FIG. 19: Constant of Dissociation determination of the m-FcγRI/hz515H7VH1D76NVL2 complex by steady-state analysis using the response at the end of the association phase versus the mouse FcγRI concentrations (400, 200, 100, 50 and 25 nM) plot.

FIG. 20: Binding of the recombinant mouse FcγRIII with hz515H7VH1D76NVL2 Mab immobilized on a CM4 sensorchip. 5 different concentrations of m-FcγRIII were tested (400, 200, 100, 50 and 25 nM).

FIG. 21: Ranking of the four Fc gamma receptors binding with hz-515H7VH1D76NVL2 Mab (one component with h-FcγRI and m-FcγRIII and two components with h-FcγRIIIA and m-FcγRI) on a constant of dissociation (in nMolar) plot in function of the half-life (in minute) of the hz515H7VH1D76NVL2 Mab/Fc gamma receptor complexes.

FIG. 22: Binding of the recombinant human FcγRI with c515H7 Mab immobilized on a CM4 sensorchip. 6 different concentrations of h-FcγRI were tested (200, 100, 50, 25, 12.5 and 6.25 nM).

FIG. 23: Binding of the recombinant human FcγRIIIA with c515H7 Mab immobilized on a CM4 sensorchip. 5 different concentrations of h-FcγRIIIA were tested (1000, 500, 250, 125 and 62.5 nM).

FIG. 24: Constant of Dissociation determination of the h-FcγRIIIA/c515H7 complex by steady-state analysis using the response at the end of the association phase versus the human FcγRIIIA concentrations (1000, 500, 250, 125 and 62.5 nM) plot.

FIG. 25: Binding of the recombinant mouse FcγRI with c515H7 Mab immobilized on a CM4 sensorchip. 5 different concentrations of m-FcγRI were tested (400, 200, 100, 50 and 25 nM).

FIG. 26: Constant of Dissociation determination of the m-FcγRI/c515H7complex by steady-state analysis using the response at the end of the association phase versus the mouse FcγRI concentrations (400, 200, 100, 50 and 25 nM) plot.

FIG. 27: Binding of the recombinant mouse FcγRIII with c515H7 Mab immobilized on a CM4 sensorchip. 5 different concentrations of m-FcγRIII were tested (400, 200, 100, 50 and 25 nM).

FIG. 28: Ranking of the four Fc gamma receptors binding with c515H7 Mab (one component with h-FcγRI and m-FcγRIII and two components with h-FcγRIIIA and m-FcγRI) on a constant of dissociation (in nMolar) plot in function of the half-life (in minute) of the c515H7 Mab/Fc gamma receptor complexes.

FIG. 29 shows antibody dependant cellular cytotoxicity (ADCC) effect of hz515H7VH1D76NVL2 (hz515H7) Mab on cells expressing CXCR4: RAMOS, DAUDI and HeLa cells.

FIG. 30 shows complement dependant cytotoxicity (CDC) effect of hz515H7VH1D76NVL2 (hz515H7) Mab on cells expressing CXCR4: DAUDI and RAMOS cells.

### EXAMPLES

### Example 1

### Generation of Monoclonal Antibodies (Mabs) Against Human CXCR4

To generate monoclonal antibodies to CXCR4, Balb/c mice were immunized with recombinant NIH3T3-CXCR4 cells and/or peptides corresponding to CXCR4 extracellular N-term and loops. The mice 6-16 weeks of age upon the first immunization, were immunized once with the antigen in complete Freund's adjuvant subcutaneously (s.c.) followed by 2 to 6 immunizations with antigen in incomplete Freund's adjuvant s.c. The immune response was monitored by retroorbital bleeds. The serum was screened by ELISA (as described bellow) and mice with the higher titers of anti-CXCR4 antibodies were used for fusions. Mice were boost intravenously with antigen two days before sacrifice and removal of the spleen.

ELISA

To select the mice producing anti-CXCR4 antibodies, sera from immunized mice was tested by ELISA. Briefly, microtiter plates were coated with purified [1-41] N-terminal peptide conjugated to BSA at 5 μg equivalent peptide/mL, 100 μL/well incubated at 4° C. overnight, then blocked with 250 μL/well of 0.5% gelatin in PBS. Dilutions of plasma from CXCR4-immunized mice were added to each well and incubated 2 hours at 37° C. The plates were washed with PBS and then incubated with a goat anti-mouse IgG antibody conjugated to HRP (Jackson Laboratories) for 1 hour at 37° C. After washing, plates were developed with TMB substrate, the reaction was stopped 5 min later by addition of 100 μL/well 1M H₂SO₄. Mice that developed the highest titers of anti-CXCR4 antibodies were used for antibody generation.

Generation of Hybridomas Producing Mabs to CXCR4

The mouse splenocytes, isolated from a Balb/c mice that developed the highest titers of anti-CXCR4 antibodies were fused with PEG to a mouse myeloma cell line Sp2/O. Cells were plated at approximately 1×10⁵/well in microtiter plates followed by two weeks incubation in selective medium containing ultra culture medium+2 mM L-glutamine+1 mM sodium pyruvate+1×HAT. Wells were then screened by ELISA for anti-CXCR4 monoclonal IgG antibodies. The antibody secreting hybridomas were then subcloned at least twice by limiting dilution, cultured in vitro to generate antibody for further analysis.

### Example 2

### Characterization by FACS Analysis of Anti-CXCR4 Mab 515117 Binding Specificity and Cancer Cell Lines Recognition

In this experiment, specific binding to human CXCR4 of anti-CXCR4 Mab 515H7 was examined by FACS analysis.

NIH3T3, NIH3T3-hCXCR4 transfected cells, MDA-MB-231, Hela and U937 cancer cell lines were incubated with 10 μg/mL of monoclonal antibody 515H7. The cells were then washed with 1% BSA/PBS/0.01% NaN3. Next, Alexa-labeled secondary antibodies were added to the cells and were allowed to incubate at 4° C. for 20 min. The cells were then washed again two times. Following the second wash, FACS analysis was performed. Results of these binding studies are provided in the following Table 6 which shows [Mean Fluorescence Intensity (WI) obtained by FACS] that anti-CXCR4 Mab 515H7 bound specifically to human CXCR4-NIH3T3 transfected cell line whereas there was no recognition on the parent NIH3T3 cells. This Mab was also able to recognize human cancer cell lines, for examples MDA-MB-231 breast cancer cells, U937 promyelocytic cancer cells and Hela cervix cancer cells.

Anti-CXCR4 Mab 515H7 recognized NIH3T3-hCXCR4 transfectant while there was no recognition of the parent NIH3T3 wild type cells. Mab 515H7 was also able to recognize cancer cell lines.

TABLE 6

| Clone | MFI on cell lines | | | | |
|---|---|---|---|---|---|
| (10 μg/ml) | NIH3T3 | NIH3T3-CXCR4 | MDA-MB-231 | Hela | U937 |
| 515H7 | 16 | 2752 | 239 | 1851 | 645 |

### Example 3

### Humanization of 515H7 Anti-CXCR4 Murine Antibody

General Procedure

Humanization of 515H7 anti-CXCR4 antibody was performed by applying the global rules of CDR-grafting. Immunogenetic analysis and definition of CDR and framework (FR) regions were performed by applying the IMGT unique numbering scheme as well as the IMGT libraries and tools (Lefranc, 1997—www.imgt.org).

The efficiency of the humanization process was evaluated by testing the functional activity of the engineered antibodies for their ability to inhibit the SDF-1-mediated recruitment of β-arrestin by a Bioluminescence Resonance Energy Transfer (BRET) assay. In this assay CXCR4 was tagged with luciferase and β-arrestin with YFP. The SDF-1 mediated recruitment of β-arrestin to CXCR4 is an important step in the signal transduction of CXCR4. Binding of humanized variants of 515H7 was also determined on a NIH3T3 cell line stably transfected with human CXCR4. The binding activity was evaluated by a competition assay with the biotinylated mouse antibody. In a second attempt, humanized antibodies were evaluated for their ability to inhibit binding of biotinylated SDF-1 to RAMOS cells. RAMOS cells were chosen because of their high expression of CXCR4 and low expression of CXCR7 and SDF-1.

These assays were used to characterize the recombinant humanized versions of anti-CXCR4 antibodies. Variable domains were formatted with human IgG1/k constant domains and cloned into the mammalian expression vector pCEP. Recombinant IgG₁/κ-derived antibodies were transiently expressed in HEK293 cells. Expression culture supernatants were filtered and antibodies were purified using protein A sepharose. Purified antibodies were re-buffered in PBS and antibodies concentrations determined by ELISA.

Humanization of 515H7 Variable Domains

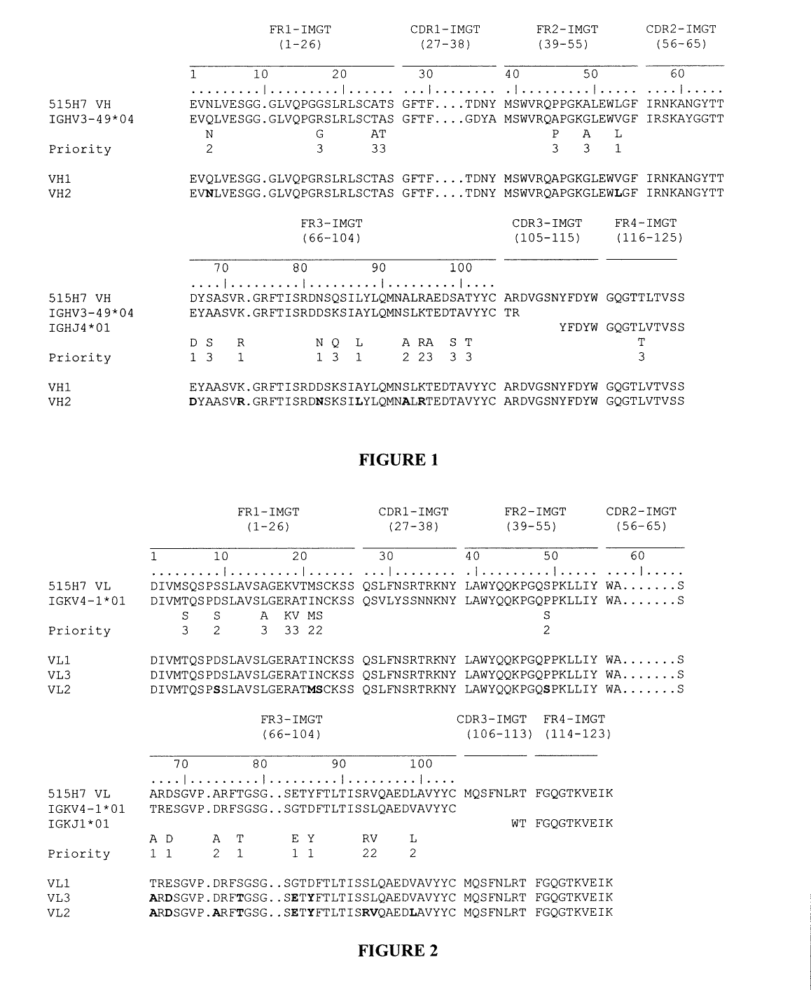

In order to select an appropriate human germline for the CDR grafting, the human germline gene with the highest homology to the 515H7 VH murine sequence was identified. With the help of IMGT databases and tools, the human IGHV3-49*04 germline gene and human IGHJ4*01 J germline gene were selected as human acceptor sequences for the murine 515H7 VH CDRs. The human V-gene IGHV3-49*04 has a homology of 80.27% to the V-gene of the variable domain of the mouse 515H7 heavy chain. The homology for the human J-gene IGHJ4*01 J is 87.50%. Nineteen residues are different between the chosen human germline genes and the VH domain of the mouse antibody 515H7. The alignment between the VH domain of the parental antibody and the germline sequences is depicted in FIG. 1.

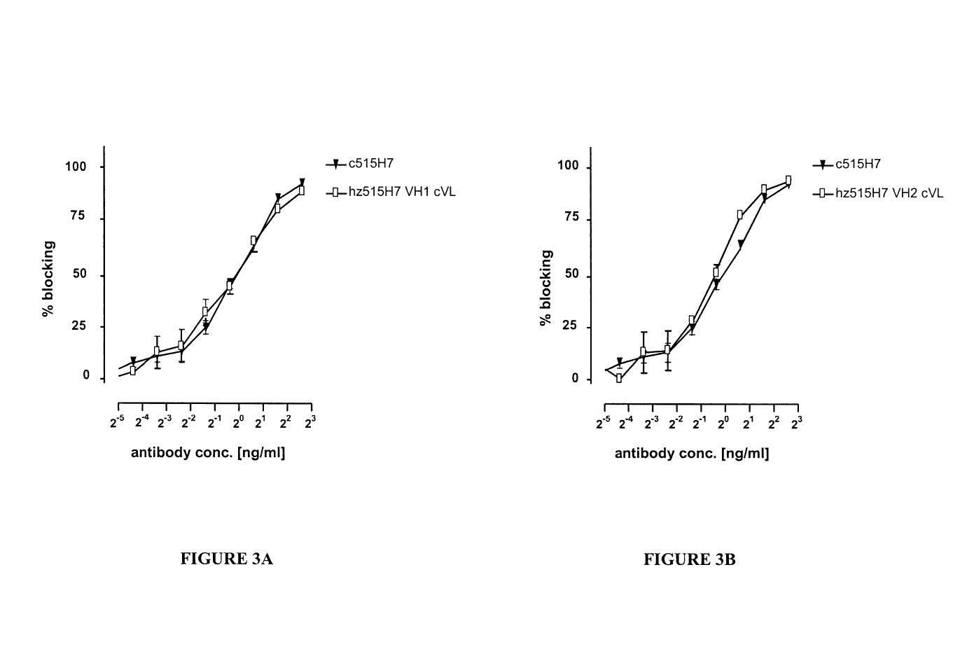

Concerning the variable domain of the light chain, the human germline genes IGKV4-1*01 and IGKJ1*01 were selected (FIG. 2). The homology with human V-gene IGKV4-1*01 is 79.12%. The 515H7 J-gene of the light chain has a homology of 84.85% to the human J-gene IGKJ1*01.

The amino acid sequence of the translated human germline genes IGHV3-49*04 and IGKV4-1*01 was used to identify homologous antibodies that have been crystallized. For the heavy chain the antibody with the accession number IMAM at the RCSB Protein Data Bank was chosen as a model, while for the light chain the antibody 1SBS was chosen. The two domains were assembled using the computer program DS visual and used as a model for the humanized antibody 515H7.

Based on the position of each residue that is different between the parental antibody and the corresponding human germline sequence, a priority rank order was given for each residue differing between the human and mouse sequences (FIGS. 1 and 2). These priorities were used to create three different variants of each humanized variable domain named VH1, VH2 and VH3, respectively.

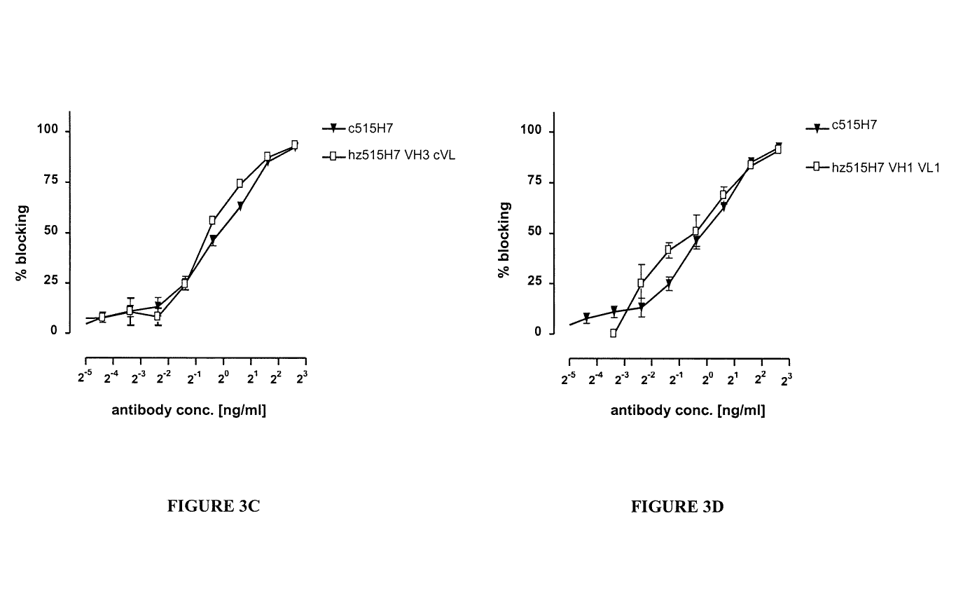

In a first series of experiments, we constructed and analysed the anti-CXCR4 binding activities of the three first humanized variants. The VH variant 1 (VH1) was combined with the murine VL and these constructs were evaluated in their capacity to inhibit the binding of a biotinylated murine 515H7 parental antibody. All constructs showed similar capacity to compete with the murine antibody (FIG. 3A-C). This indicates that the most human VH variant has the same binding capacity as the lesser human variants. Therefore, VH1 was combined with the three different variants of VL (FIG. 3D-F). Only the combination of VH1 and VL3 showed a reduced capacity to compete with the biotinylated murine antibody, while the most human variant VH1 VL1 that carries no back mutations in the frameworks showed the same cross blocking activity as the chimeric antibody.

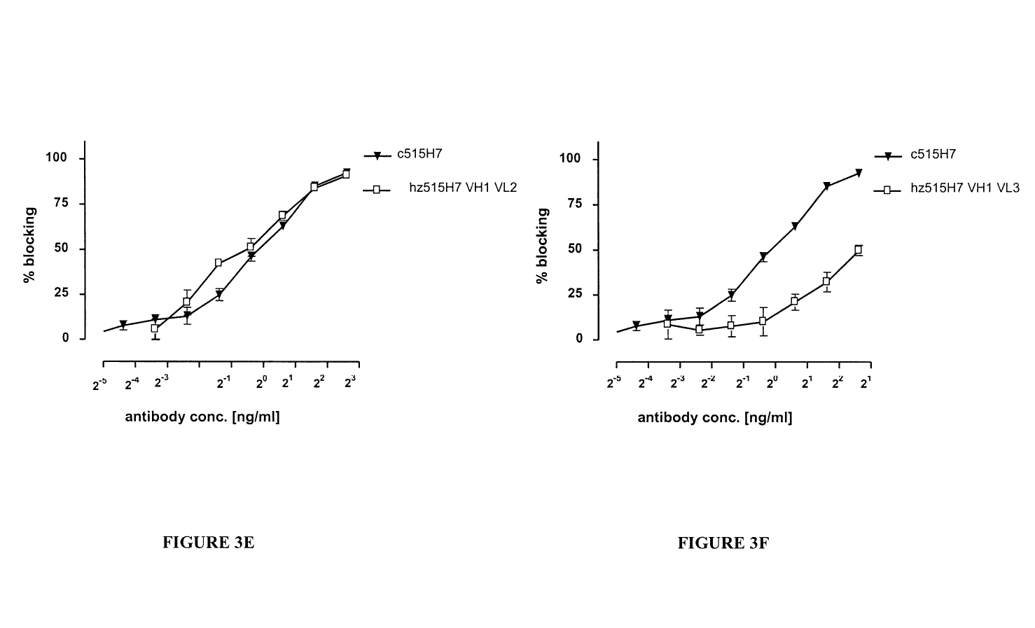

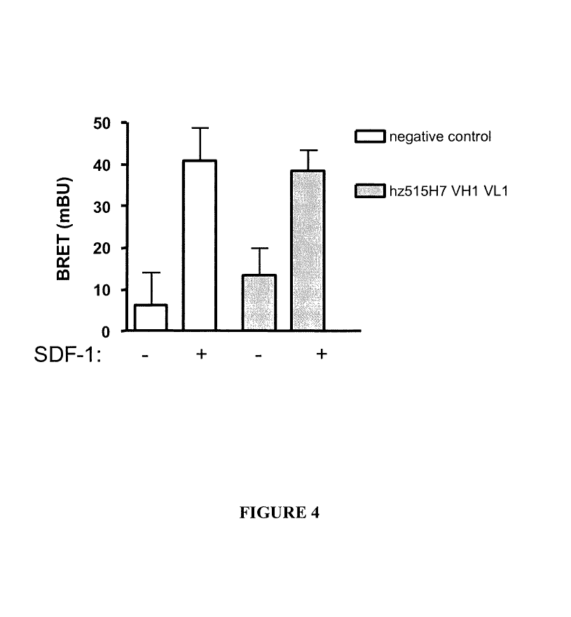

This variant VH1 VL1 was further tested for its capacity to inhibit SDF-1 mediated recruitment of β-arrestin in BRET assays (FIG. 4). Despite desirable binding activity to the receptor as determined by cross blocking of the parental antibody, the construct VH1 VL1 showed only a weak inhibition of the recruitment of β-arrestin. This lack of strong inhibitory activity makes substitution of human framework residues with murine residues necessary. Single back mutations were constructed for the VH 1. The following residues were substituted: V48L, E61D, D76N and A81L (numbering according to the primary amino acid sequence). These single back mutants of the variant VH1 were combined with the variant VL1. Of these only the back mutation D76N led to an increased inhibition of the signal transduction as evaluated by BRET assay (FIG. 5B).

To increase the activity of this construct and further evaluate the importance of other residues different double back mutants were constructed for the VH 1. The inhibitory activity of these constructs was slightly improved (average inhibition of about 45-50%), but not satisfactory (FIG. 5C). The single back mutant D76N was then combined with the three different VL variants (FIG. 5D). The construct hz515H7 VH D76N VL2 showed an activity of 88.2% on average which is in the same range as the chimeric antibody.

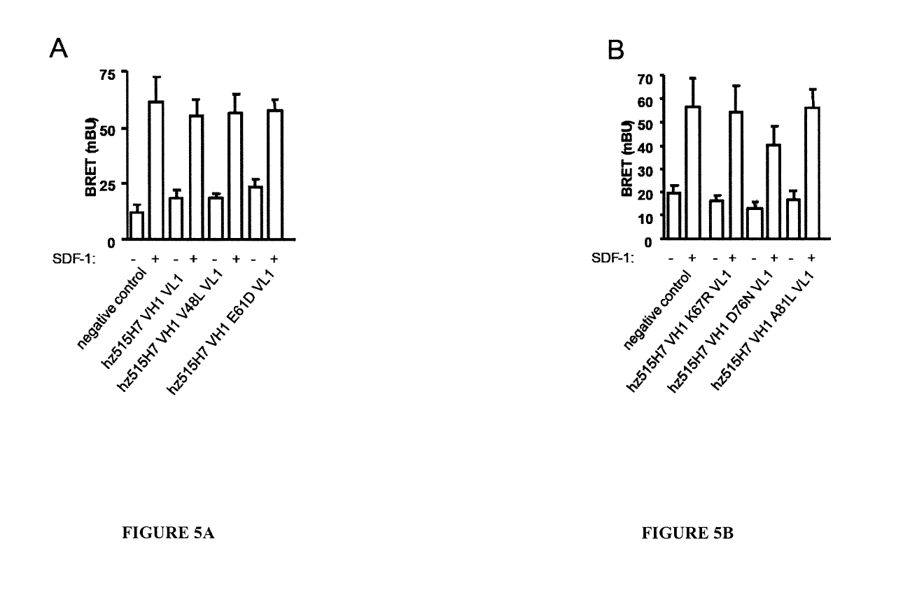

Single and double back mutations were constructed in the variant VL1 domain and compared to the activity of the construct hz515H7 VH1 D76N VL2 (FIG. 6). None of the tested combinations had a similar or better activity as this construct.

The percentage of human residues in the framework was calculated for hz515H7 VH1 D76N VL2: it contains 14 non-human residues out of 180 residues, which equals a <<germinality index>> of 92.2%. By way of comparison, the humanized and marketed antibodies bevacizumab and trastuzumab contain respectively 30 and 14 non-human residues in their variable domains.

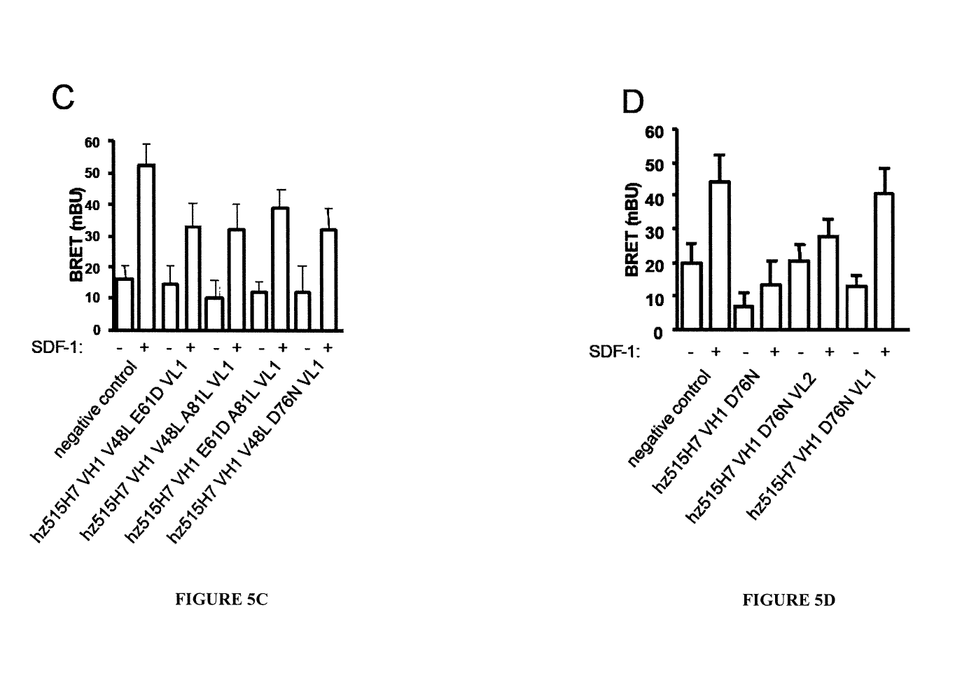

The four best humanized forms, showing the strongest efficacy to inhibit SDF-1-mediated β-arrestin recruitment were also tested for their capacity to inhibit the binding of biotinylated SDF-1 (FIG. 7A). A close correlation of inhibition of SDF-1 binding and β-arrestin recruitment was determined. This correlation indicates that the inhibition of SDF-1 binding is most likely the main mechanism of the inhibition of the signal transduction.

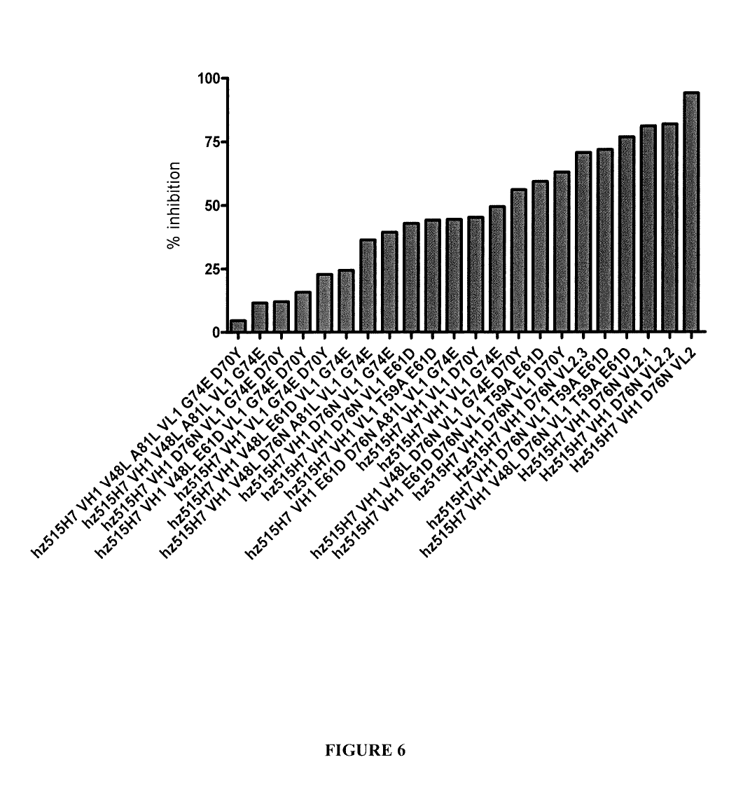

In order to further humanize the hz515H7 VL2 variant, three additional variants were designed, by using the information gained with the double and triple mutants evaluated in FIG. 6. Four and five additional residues were humanized in respectively variant VL2.1, VL2.2 and VL2.3 (also referred as VL2-1, VL2-2 and VL2-3). They correspond to the residues D9, P49, D66, S69, S83, L84; V89. An alignment of these three variants in comparison with VL2 is shown FIG. 8.

The capacity of these VL2 variants to inhibit the SDF-1 mediated recruitment of β-arrestin was evaluated. The humanized hz515H7 VH D76N VL2, VL2.1, VL2.2 and VL2.3 variants showed an activity similar to the chimeric antibody c515H7 (FIG. 6).

### Example 4

### Characterization by FACS Analysis of Anti-CXCR4 Humanized Mabs 515117 Binding Specificity and Cancer Cell Line Recognition

In this experiment, specific binding to human CXCR4 of anti-CXCR4 humanized Mabs 515H7 was examined by FACS analysis.

NIH3T3, NIH3T3-hCXCR4 transfected cells and Ramos, U937 cancer cell lines were incubated with 0 to 10 μg/mL of humanized Mabs 515H7 (hz515H7 VH1 D76N VL2, hz515H7 VH1 D76N VL2.1, hz515H7 VH1 D76N VL2.2 and hz515H7 VH1 D76N VL2.3) for 20 min at 4° C. in the dark in 100 μl Facs buffer. After 3 washing in Facs buffer, cells were incubated with the secondary antibody, a goat anti-human Alexa 488 (dilution 1/500), for 20 minutes at 4° C. in the dark. After 3 washing in Facs buffer, propidium iodide was added in each well and only viable cells were analyzed by Facs. At least 5000 viable cells were assessed to evaluate the mean value of fluorescence intensity for each condition.

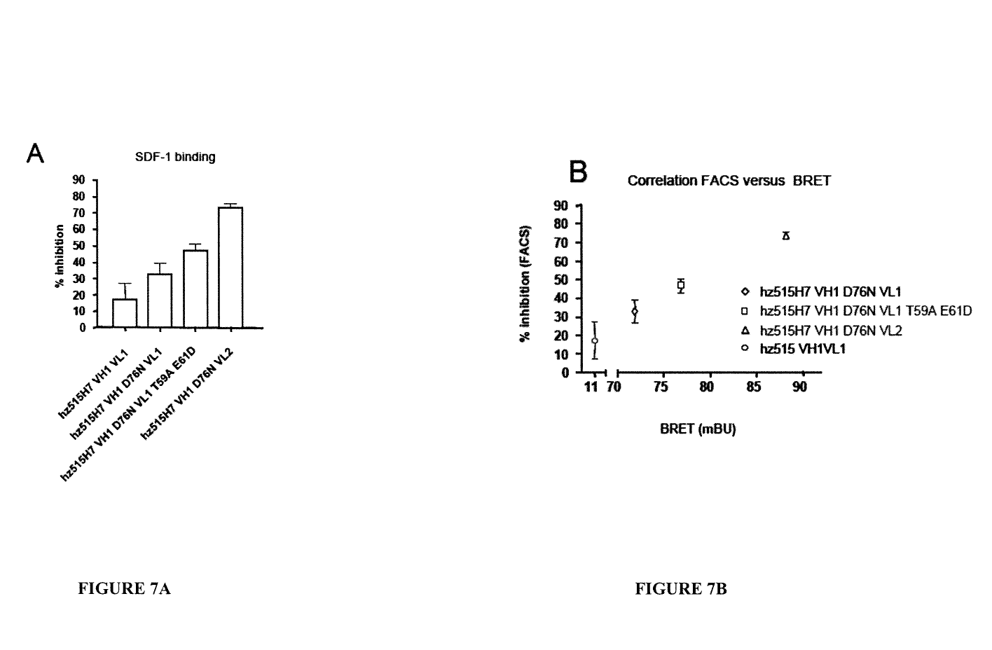

Results of these binding studies are provided in FIGS. 9A-9C which show [Mean Fluorescence Intensity (MFI) obtained by FACS] that anti-CXCR4 humanized Mabs hz515H7 bound specifically to human CXCR4-NIH3T3 transfected cell line (FIG. 9A) (MFI=2.2 with NIH3T3 parent cells) and also recognize human cancer cell lines, for example U937 (FIG. 9B) and Ramos (FIG. 9C).

### Example 5

### Antibody Dependent Cellular Cytotoxicity (ADCC) Effect of hz515H7VH1D76NVL2 Mab on Cells Expressing CXCR4

ADCC was measured by a lactate dehydrogenase (LDH) releasing assay using the Cytotoxicity Detection Kit^{PLUS} (Roche Applied Science, Indianapolis, Ind., USA) according to the manufacturer's instructions. Lactate dehydrogenase is a soluble cytosolic enzyme that is released into the culture medium following loss of membrane integrity resulting from either apoptosis or necrosis. LDH activity, therefore, can be used as an indicator of cell membrane integrity and serves as a general means to assess cytotoxicity, including ADCC.

Peripheral blood mononuclear cells (PBMC) were isolated from human buffy coats obtained from healthy donors, using a Ficoll density gradient (Ficoll-Paque PLUS, GE Healthcare, Amersham, UK). Natural Killer (NK) cells were separated from the PBMC fraction according to the RoboSep® Human NK Cell Enrichment Kit manufacturer's protocol (StemCell Technologies). NK cells were plated in 96-well flat bottom plates at an effector-to-target ratio of 50:1 at 50 μL per well. 10000 Target cells (Ramos), pre-incubated with antibodies at room temperature for 10 min, were added on effector cells at 50 μL/well. After incubation for 4 h at 37° C., the cytotoxicity was determined by measuring the amount of LDH released. Percent of cytotoxicity was calculated as follows: % lysis=[experimental release−effector and target spontaneous release]/[target maximum release−target spontaneous release]×100.

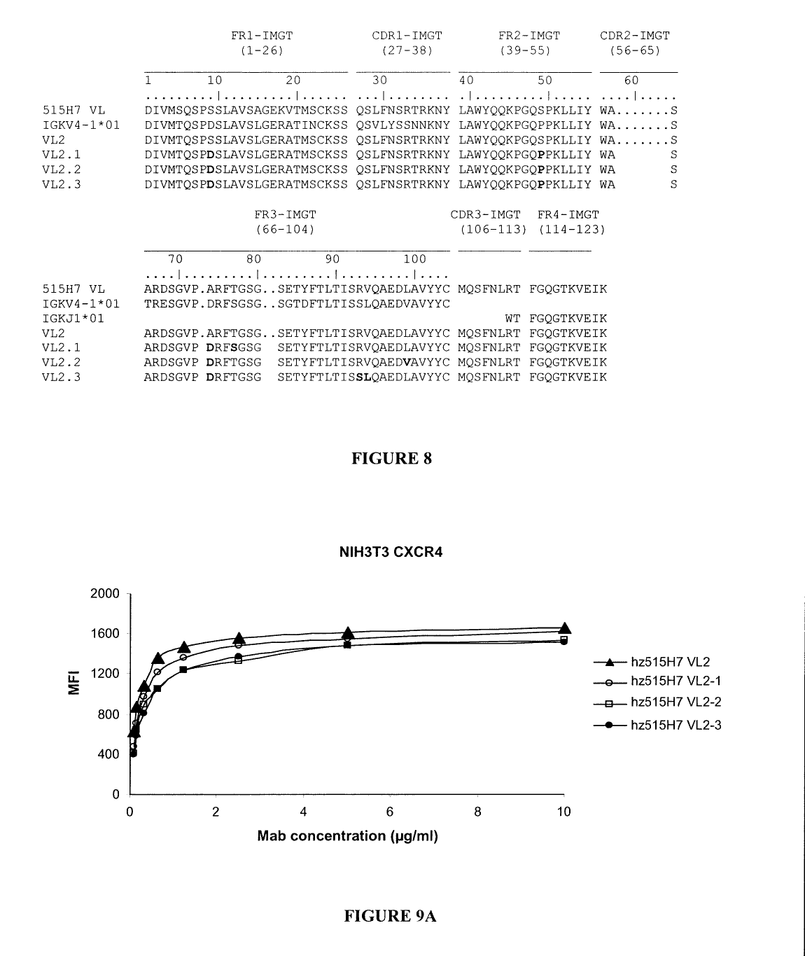

FIG. 10 shows ADCC on Ramos cells expressing high level of CXCR4 and on NK cells alone [CXCR4 levels (MFI): Ramos>NK cells]. Black columns: Hz515H7VH1D76NVL2 (hz515H7VL2) (10 μg/mL), white columns: isotype control hIgG1 (10 μg/mL). No effect was observed when cells were incubated with the hIgG1 isotype control (FIGS. 10A and 10B). In contrast, hz515H7VH1D76NVL2 Mab was able to induce significant ADCC (47.9%+/−8.9) on Ramos cells (FIG. 10A) whereas there was no significant ADCC (3%+/−3) on NK cells expressing low level of CXCR4 (FIG. 10B).

### Example 6

### Antibody Dependent Cellular Cytotoxicity (ADCC) Effect of c515H7 Mab on Cells Expressing CXCR4

ADCC was measured by a lactate dehydrogenase (LDH) releasing assay using the Cytotoxicity Detection Kit^{PLUS} (Roche Applied Science, Indianapolis, Ind., USA) according to the manufacturer's instructions.

Peripheral blood mononuclear cells (PBMC) were isolated from human buffy coats obtained from healthy donors, using a Ficoll density gradient (Ficoll-Paque PLUS, GE Healthcare, Amersham, UK). Natural Killer (NK) cells were separated from the PBMC fraction according to the RoboSep® Human NK Cell Enrichment Kit manufacturer's protocol (StemCell Technologies). NK cells were plated in 96-well flat bottom plates at an effector-to-target ratio of 50:1 at 50 μL per well. 10000 Target cells (Ramos), pre-incubated with antibodies at room temperature for 10 min, were added on effector cells at 50 μL/well. After incubation for 4 h at 37° C., the cytotoxicity was determined by measuring the amount of LDH released. Percent of cytotoxicity was calculated as follows: % lysis=[experimental release−effector and target spontaneous release]/[target maximum release−target spontaneous release]×100.

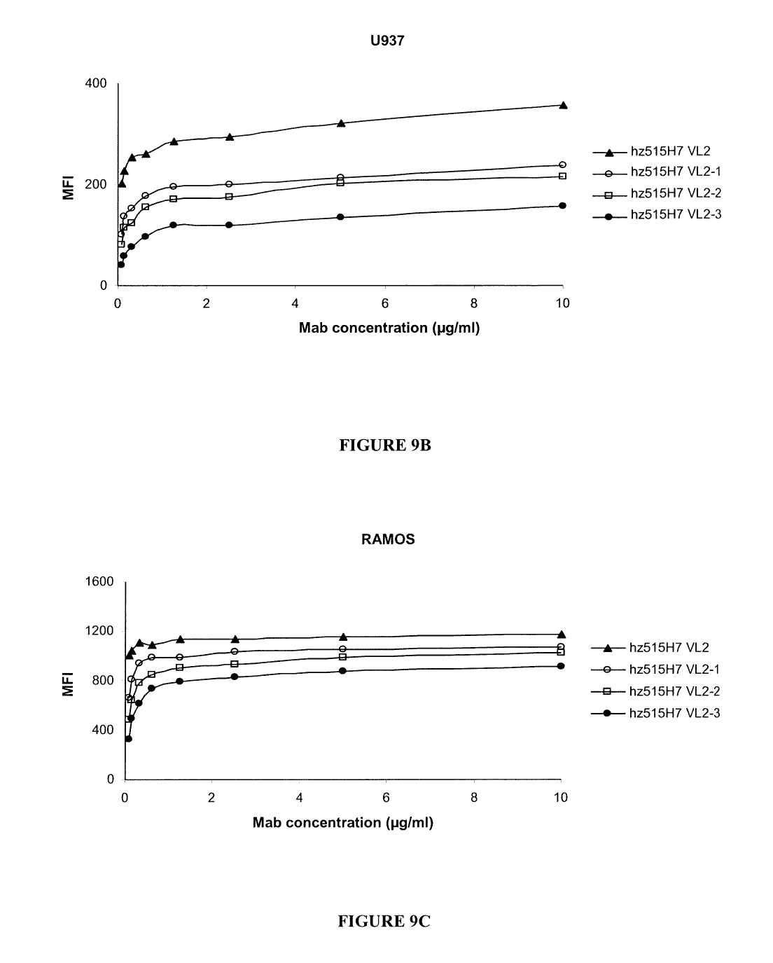

FIG. 11 shows ADCC on Ramos cells expressing high level of CXCR4 and on NK cells alone [CXCR4 levels (MFI): Ramos>NK cells]. Black columns: c515H7 (10 μg/mL), white columns: isotype control hIgG1 (10 μg/mL). No effect was observed when cells were incubated with the hIgG1 isotype control (FIGS. 11A and 11B). In contrast, c515H7 Mab was able to induce significant ADCC (61.4%+/−8.1) on Ramos cells (FIG. 11A) whereas there was no significant ADCC (5.4%+/−4.6) on NK cells expressing low level of CXCR4 (FIG. 11B).

### Example 7

Complement Dependent Cytotoxicity (CDC) Effect of hz515H7VH1D76NVL2 Mab on Cells Expressing CXCR4

CDC assay was based on ATP measurement using CellTiter Glo reagent (Promega, Madison, Wis., USA).

Briefly, 10000 target cells were plated in 96-well flat bottom plates in presence of hz515H7VH1D76NVL2. Following incubation at room temperature for 10 minutes, pooled human serum from healthy donors was added at a final concentration of 10%. After 1 h at 37° C., viability was determined by measuring the amount of ATP. Percent of cytotoxicity was calculated as follows: % Cytotoxicity=100−[[experimental/target cell without antibody]×100].

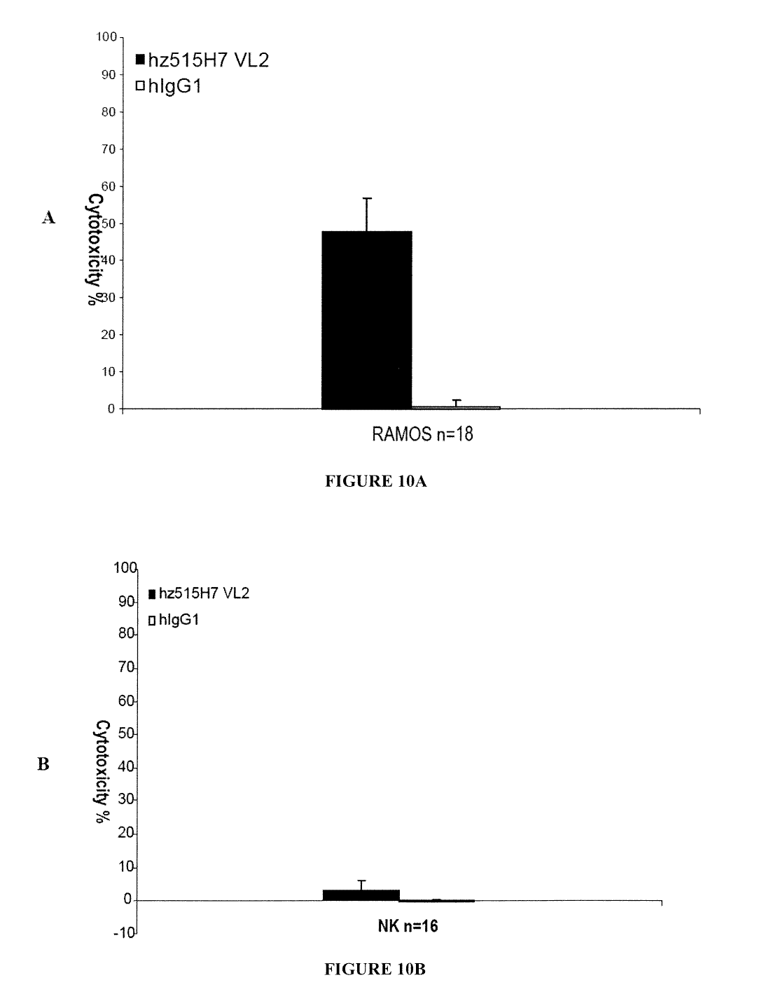

FIG. 12 shows CDC on Ramos and NIH3T3-CXCR4 cell lines expressing high levels of CXCR4. Black columns: Hz515H7VH1D76NVL2 (hz515H7VL2) (10 μg/mL), white columns: isotype control hIgG1 (10 μg/mL). No effect was observed when cells were incubated with the hIgG1 isotype control (FIG. 12). In contrast, hz515H7VH1D76NVL2 Mab was able to induce significant CDC (around 80%) on both NIH/3T3CXCR4 and RAMOS cell lines (FIG. 12).

### Example 8

Complement Dependent Cytotoxicity (CDC) Effect of c515H7 Mab on Ramos Cells Expressing High Level of CXCR4

CDC assay was based on ATP measurement using CellTiter Glo reagent (Promega, Madison, Wis., USA).

Briefly, 10 000 Ramos cells were plated in 96-well flat bottom plates in presence of Mabs. Following incubation at room temperature for 10 minutes, pooled human serum from healthy donors was added at a final concentration of 10%. After 1 h at 37° C., viability was determined by measuring the amount of ATP. Percent of cytotoxicity was calculated as follows: % Cytotoxicity=100−[[experimental/target cell without antibody]×100].

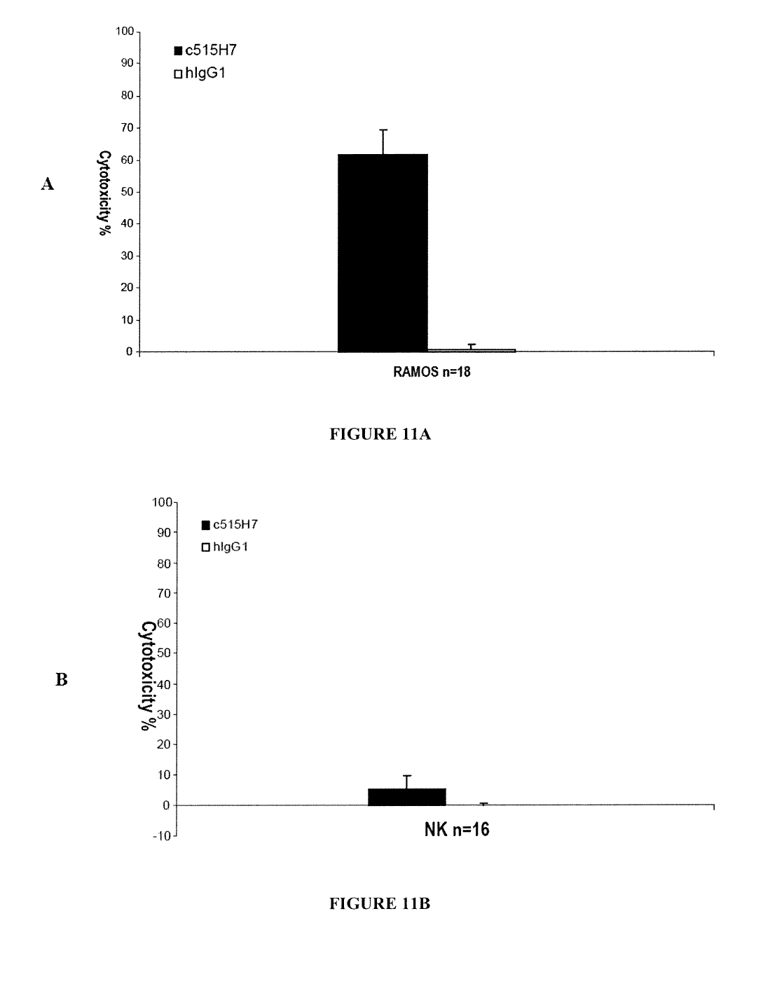

FIG. 13 shows CDC on Ramos cell line expressing high level of CXCR4. Black columns: c515H7 (10 μg/mL), white columns: isotype control hIgG1 (10 μg/mL). No effect was observed when cells were incubated with the hIgG1 isotype control (FIG. 13). In contrast, c515H7 Mab was able to induce significant CDC (34%) on RAMOS cells (FIG. 13)

### Example 9

### Complement Dependent Cytotoxicity (CDC) Dose Effect of hz515H7VH1D76NVL2 and c515H7 Mabs on Ramos Cells Expressing High Level of CXCR4

CDC assay was based on ATP measurement using CellTiter Glo reagent (Promega, Madison, Wis., USA).

Briefly, 10 000 Ramos cells were plated in 96-well flat bottom plates in presence of Mabs. Following incubation at room temperature for 10 minutes, pooled human serum from healthy donors was added at a final concentration of 10%. After 1 h at 37° C., viability was determined by measuring the amount of ATP. Percent of cytotoxicity was calculated as follows: % Cytotoxicity=100−[[experimental/target cell without antibody]×100].

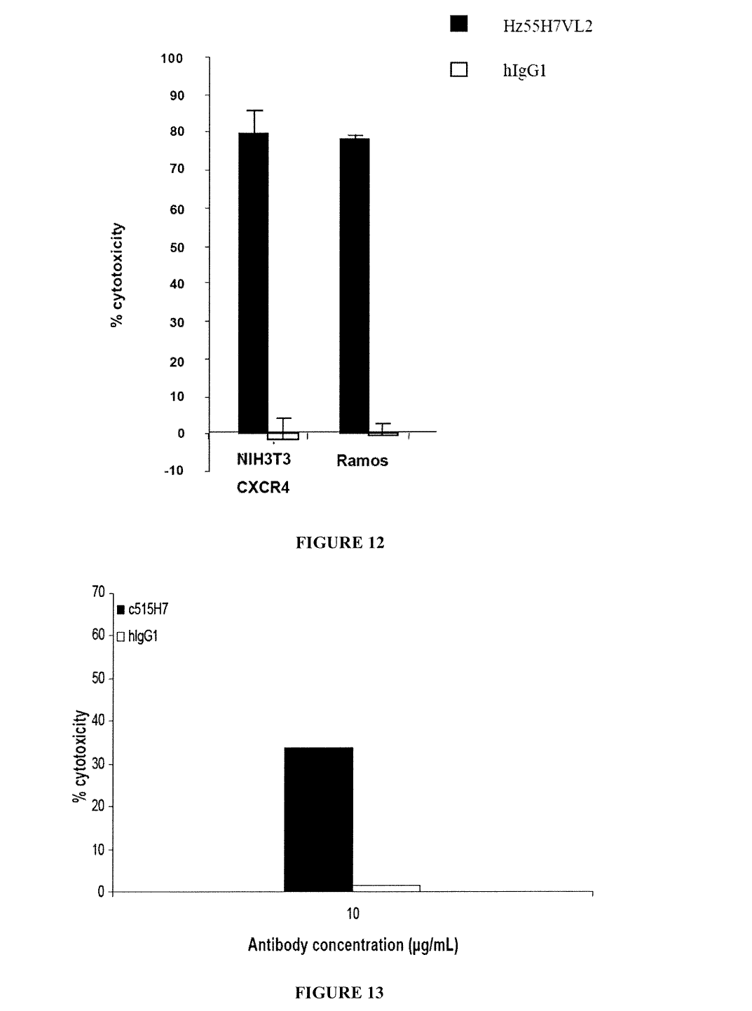

FIG. 14 show CDC on Ramos cell line expressing high level of CXCR4. Black columns: either hz515H7VH1D76NVL2 (hz515H7VL2) (FIG. 14A) or c515H7 (FIG. 14B) (10 μg/mL), white columns: isotype control hIgG1 (10 μg/mL). No effect was observed when cells were incubated with the hIgG1 isotype control (FIGS. 14A and 14B). In contrast, hz515H7VH1D76NVL2 (FIG. 14A) and c515H7 (FIG. 14B) Mabs were able to induce significant CDC on Ramos cells with CDC max of 74% and 34%, respectively, with EC₅₀ of 0.033 μg/mL and 0.04 μg/mL, respectively.

### Example 10

### Study of the Interaction Between hz515H7VH1D76NVL2 Mab and h-FcγRI, h-FcγRIIIA, m-FcγRI and m-FcγRIII by Real Time Surface Plasmon Resonance

The experiments were carried out using a Biacore X device. The soluble forms of the four Fc □gamma receptors used in this study were purchased from R&D Systems:

1—Recombinant human FcγRI [CD64] corresponds to the Gln16-Pro288 fragment with a C-terminal 6-His tag [catalog number: 1257-FC]. The molecular weight of 50 kDa (specified by the supplier) used in this study corresponds to the mean of the molecular weight defined by SDS-PAGE in reducing condition.

2—Recombinant human FcγRIIIA variant V [CD16a] corresponds to the Gln17-Gln208 fragment with a C-terminal 6-His tag [catalog number: 4325-FC]. The molecular weight of 45 kDa (specified by the supplier) used in this study corresponds to the mean of the molecular weight defined by SDS-PAGE in reducing condition.

3—Recombinant mouse FcγRI [CD64] corresponds to the Gln25-Pro297 fragment with a C-terminal 6-His tag [catalog number: 2074-FC]. The molecular weight of 55 kDa (specified by the supplier) used in this study corresponds to the mean of the molecular weight defined by SDS-PAGE in reducing condition.

4—Recombinant mouse FcγRIII [CD16] corresponds to the Ala31-Thr215 fragment with a C-terminal 10-His tag [catalog number: 1960-FC]. The molecular weight of 37.5 kDa (specified by the supplier) used in this study corresponds to the mean of the molecular weight defined by SDS-PAGE in reducing condition.

The other reagents were supplied by Biacore (GE Healthcare).

1964 RU of hz515H7VH1D76NVL2 Mab were immobilized using the amine coupling kit chemistry on the second flowcell (FC2) of a CM4 sensorchip. The first flowcell (FC1) activated by NHS and EDC mixture and des-activated by ethanolamine served as the reference surface to check and subtract the non specific interaction between the analyte (Fc gamma receptors) and the sensorchip matrix.

The kinetic experiments were carried out at 25° Celsius at a flow rate of 30 μl/min. The HBS-EP buffer was used either as the running buffer or for the preparation of analyte solutions. The analyte solutions were injected during 90 seconds (association phase) with a 90 seconds delay (dissociation phase). An injection of running buffer as analyte was used as a double reference. All the sensorgrams were corrected by this double reference sensorgram.

After each injection of the analyte, the sensorchip was regenerated by injection of either 20 mM NaOH solution after h-FcγRI and m-FcγRIII or 10 mM NaOH after h-FcγRIIIA and m-FcγRI.

Two mathematical models were used to analyze the sensorgrams: the “Langmuïr” and the “heterogeneous ligand” models.

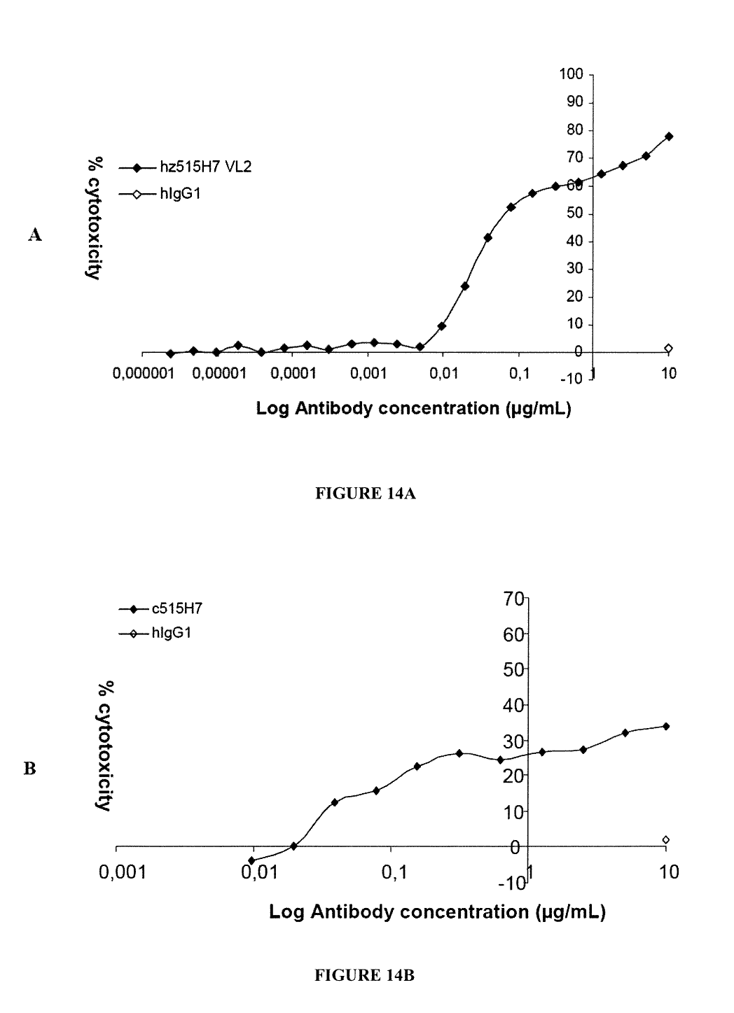

Sensorgrams obtained with h-FcγRI [FIG. 15] were not perfectly fitted by the Langmuïr model (5%<Chi2/Rmax<20%) but the “heterogeneous ligand” model did not improve the quality of the fitting. Using the Langmuïr model, the constant of dissociation was in the nanomolar range (0.9±0.1 nM).

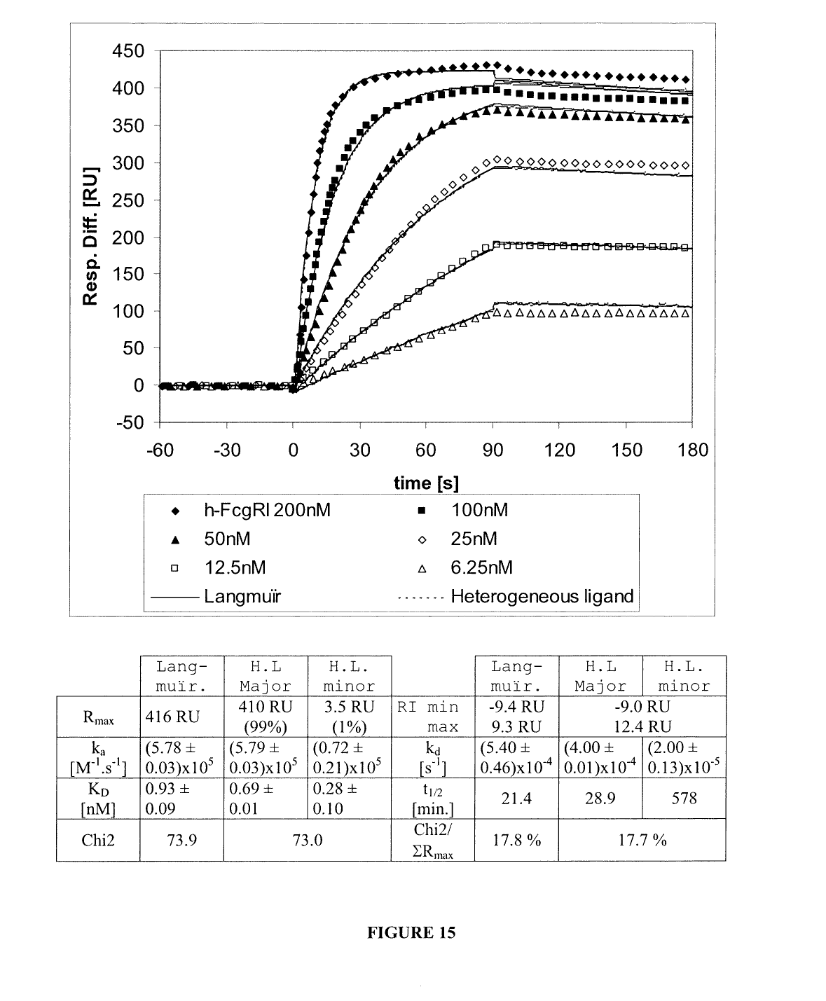

Sensorgrams obtained with h-FcγRIIIA [FIG. 16] were clearly not fitted by the Langmuïr model (Chi2/Rmax>20%). The “heterogeneous ligand” model improved significantly the quality of the fitting (Chi2/Rmax<5%). According to this model, the hz515H7VH1D76NVL2 Mab Fc domain may be regarded as a mixture of two components. The major one representing 79% of the total amount showed a constant of dissociation between 300 and 350 nM, the minor one (21%) showed a constant of dissociation between 27 and 32 nM. According to the literature, the heterogeneity observed with h-FcγRIIIA was probably linked to the glycosylation heterogeneity on the Mab Fc domain.

A plot representing a mean of the response in RU (close to Req) at the end of the association phase versus the h-FcγRIIIA concentration (C) can be fitted with the mathematical model:

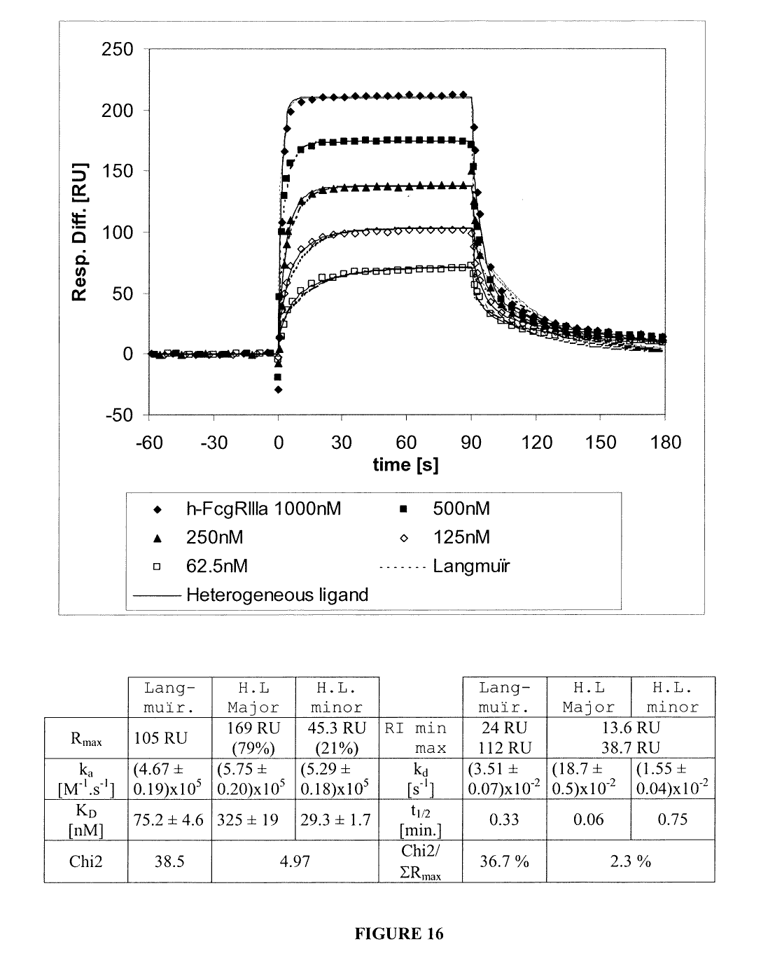

Req=(K_{A}·C·Rₘₐₓ 1/K_{A}·C·n+1), with n=1 [FIG. 17]. The constant of dissociation K_{D} corresponding to 1/K_{A} is the equal to 176 nM.

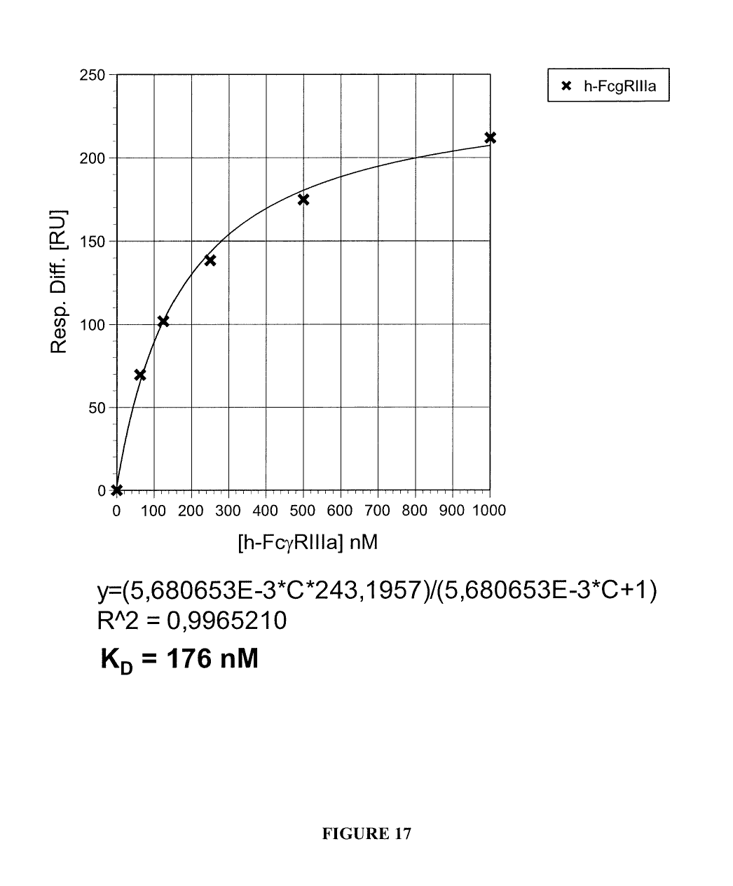

Sensorgrams obtained with m-FcγRI [FIG. 18] may be fitted by the Langmuïr model (5%<Chi2/Rmax<10%) but the “heterogeneous ligand” model improved significantly the quality of the fitting (Chi2/Rmax<1%). According to this model, the hz515H7VH1D76NVL2 Mab Fc domain may be regarded as a mixture of two components. The major one representing 82% of the total amount showed a constant of dissociation between 75 and 80 nM, the minor one (18%) showed a constant of dissociation around 90 nM. Even if the constant of dissociation were close, the kinetics rates were significantly different (the association rate was 5.7 time better for the major component but its dissociation rate was 4.8 time quicker).

A plot representing a mean of the response in RU (close to Req) at the end of the association phase versus the m-FcγRI concentration (C) can be fitted with the mathematical model:

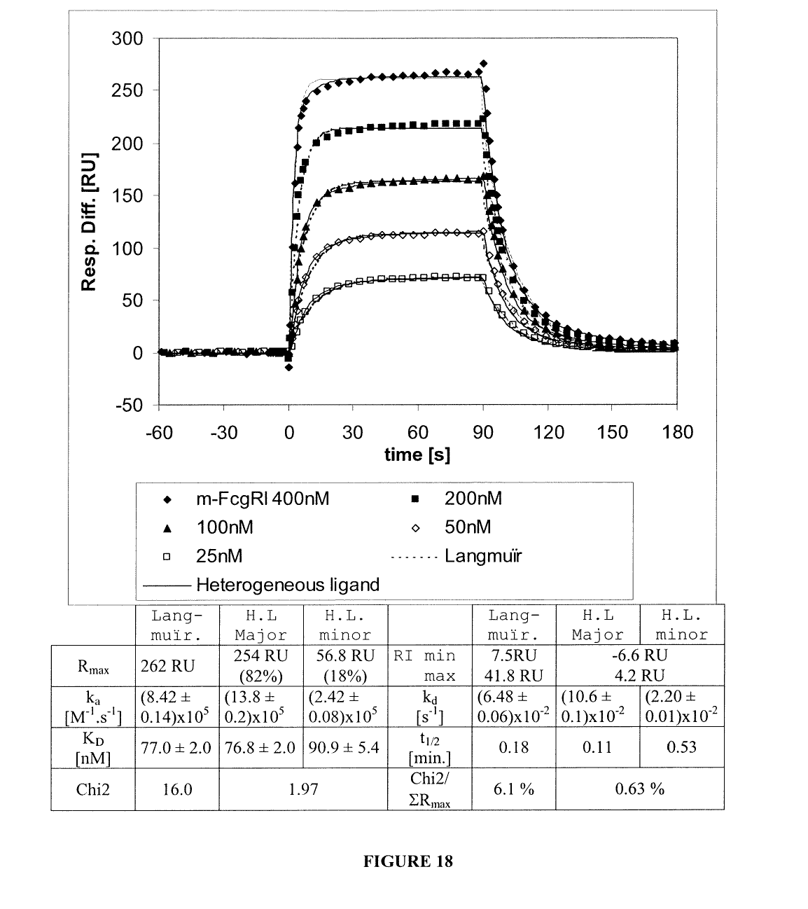

Req=(K_{A}·C·Rₘₐₓ)/(K_{A}·C·n+1) with n=1 [FIG. 19]. The constant of dissociation K_{D} corresponding to 1/K_{A} is the equal to 95 nM.

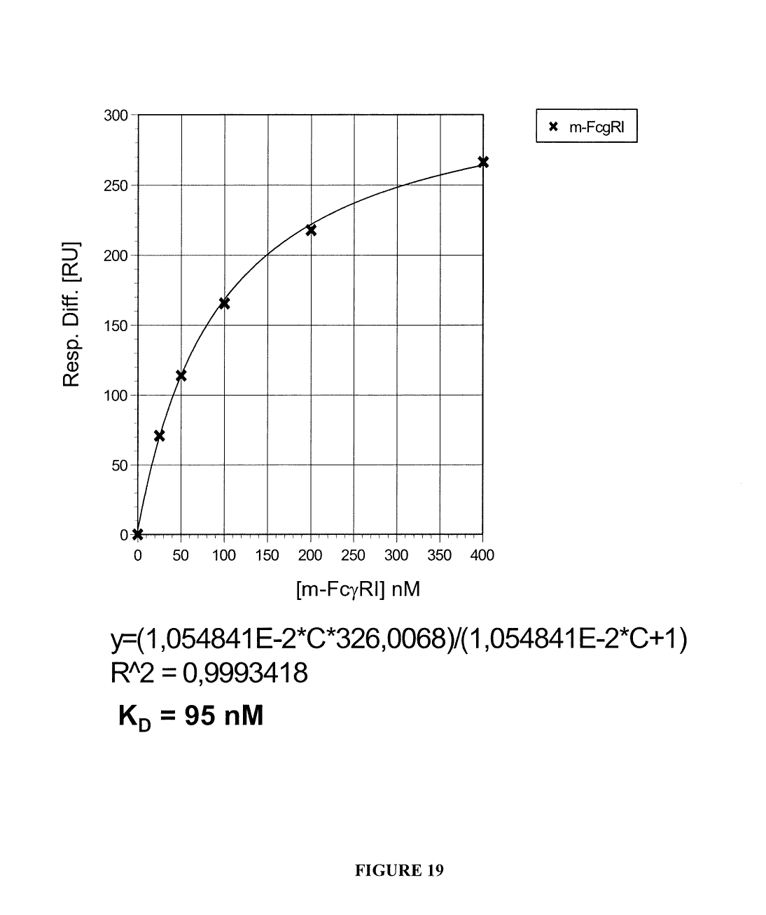

Sensorgrams obtained with m-FcγRIII [FIG. 20] were not perfectly fitted by the Langmuïr model (5%<Chi2/Rmax<20%) but the “heterogeneous ligand” model did not improve the quality of the fitting. Using the Langmuïr model the constant of dissociation was around 17 and 18 nM.

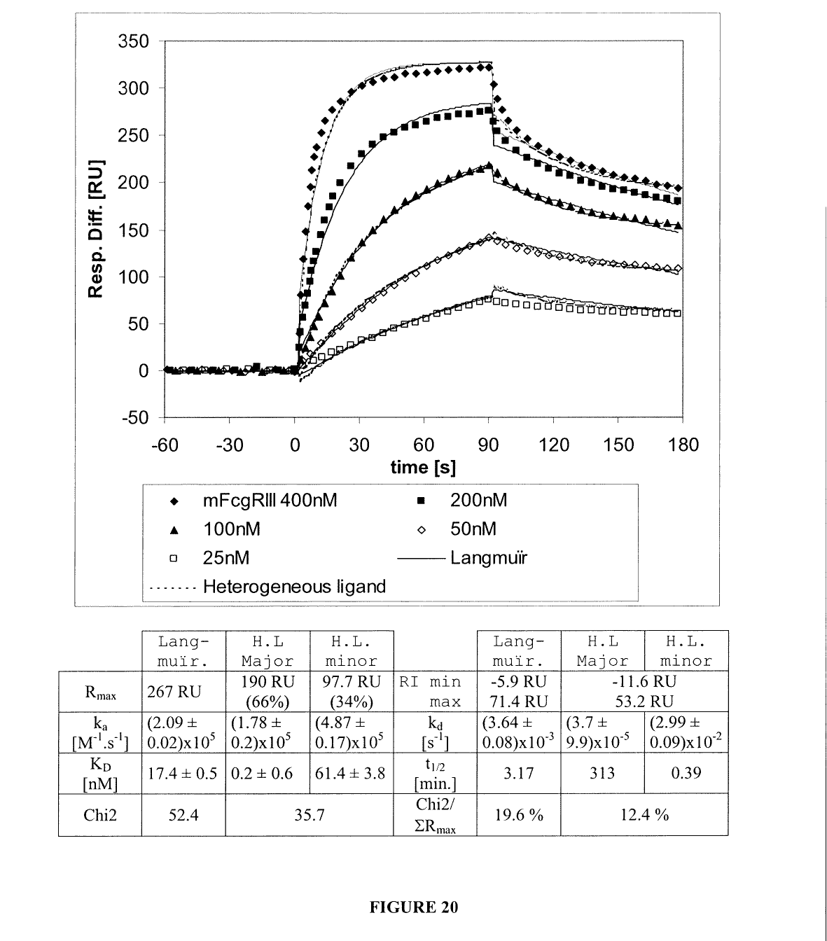

A ranking of the four Fc gamma receptors is presented in FIG. 21 representing Kd plot in function of the half-life of the complex. In accordance with the literature, h-FcγRI binds with high affinity and h-FcγRIIIA with a lower affinity to the Fc part of a human IgG1 isotype antibody. m-FcγRIII binds with an intermediate affinity between the affinity of the major component of hz-515H7VH1D76NVL2 Mab for h-FcγRIIIA and the affinity of h-FcγRI. Both components of the hz515H7VH1D76NVL2 Mab interact with m-FcγRI with an intermediate affinity between the affinities of both components of hz515H7VH1D76NVL2 for h-FcγRIIIA

These experiments clearly showed that the hz515H7VH1D76NVL2 Mab Fc domain interacts significantly with the four FcγR tested.

### Example 11

### Study of the Interaction Between c515H7 Mab and h-FcγRI, h-FcγRIIIA, m-FcγRI and m-FcγRIII by Real Time Surface Plasmon Resonance

The experiments were carried out using a Biacore X device. The soluble forms of the four Fc □gamma receptors used in this study were purchased from R&D Systems:

1—Recombinant human FcγRI [CD64] corresponds to the Gln16-Pro288 fragment with a C-terminal 6-His tag [catalog number: 1257-FC]. The molecular weight of 50 kDa (specified by the supplier) used in this study corresponds to the mean of the molecular weight defined by SDS-PAGE in reducing condition.

2—Recombinant human FcγRIIIA variant V [CD16a] corresponds to the Gln17-Gln208 fragment with a C-terminal 6-His tag [catalog number: 4325-FC]. The molecular weight of 45 kDa (specified by the supplier) used in this study corresponds to the mean of the molecular weight defined by SDS-PAGE in reducing condition.

3—Recombinant mouse FcγRI [CD64] corresponds to the Gln25-Pro297 fragment with a C-terminal 6-His tag [catalog number: 2074-FC]. The molecular weight of 55 kDa (specified by the supplier) used in this study corresponds to the mean of the molecular weight defined by SDS-PAGE in reducing condition.

4—Recombinant mouse FcγRIII [CD 16] corresponds to the Ala31-Thr215 fragment with a C-terminal 10-His tag [catalog number: 1960-FC]. The molecular weight of 37.5 kDa (specified by the supplier) used in this study corresponds to the mean of the molecular weight defined by SDS-PAGE in reducing condition.

The other reagents were supplied by Biacore (GE Healthcare).

2017 RU of c515H7 Mab were immobilized using the amine coupling kit chemistry on the second flowcell (FC2) of a CM4 sensorchip. The first flowcell (FC1) activated by NHS and EDC mixture and des-activated by ethanolamine served as the reference surface to check and subtract the non specific interaction between the analyte (Fc gamma receptors) and the sensorchip matrix.

The kinetic experiments were carried out at 25° Celsius at a flow rate of 30 μl/min. The HBS-EP buffer was used either as the running buffer or for the preparation of analyte solutions. The analyte solutions were injected during 90 seconds (association phase) with a 90 seconds delay (dissociation phase). An injection of running buffer as analyte was used as a double reference. All the sensorgrams were corrected by this double reference sensorgram.

After each injection of the analyte, the sensorchip was regenerated by injection of 20 mM NaOH, 75 mM NaCl solution.

Two mathematical models were used to analyze the sensorgrams: the “Langmuïr” and the “heterogeneous ligand” models.

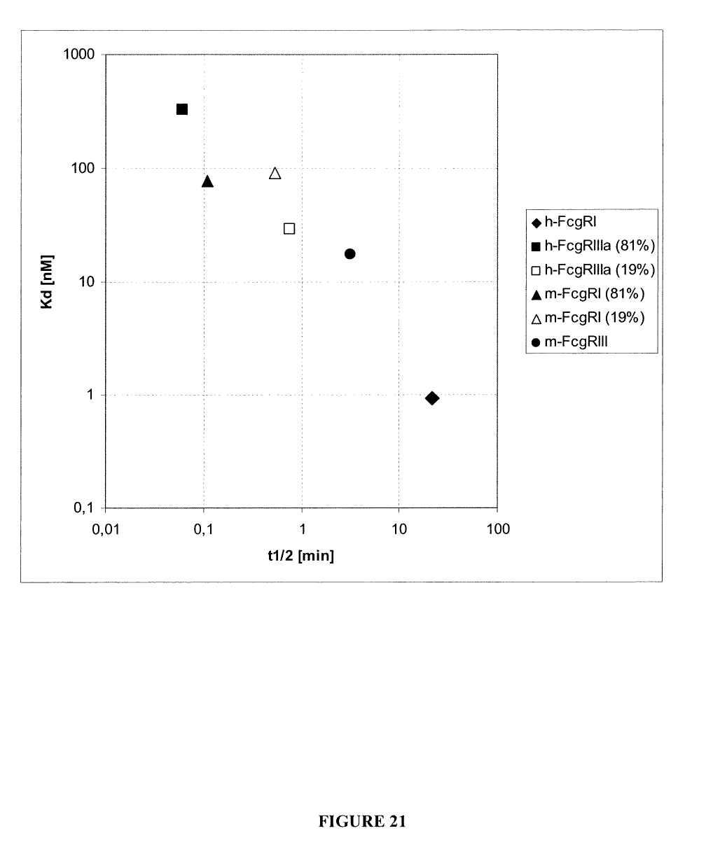

Sensorgrams obtained with h-FcγRI [FIG. 22] were not perfectly fitted by a Langmuïr model (Chi2/Rmax>10%) but the “heterogeneous ligand” model did not improve the quality of the fitting. Using the Langmuïr model, the constant of dissociation was close to the nanomolar range (1.2±0.1 nM).

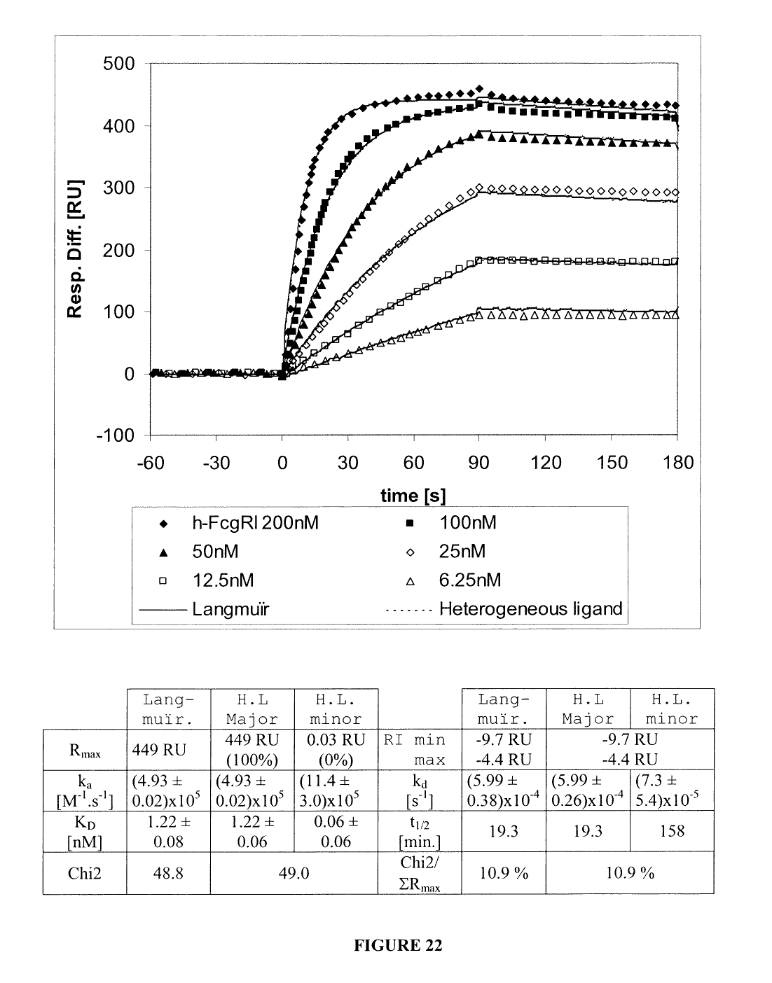

Sensorgrams obtained with h-FcγRIIIA [FIG. 23] were clearly not fitted by a Langmuïr model (Chi2/Rmax>20%). The “heterogeneous ligand” model improved significantly the quality of the fitting (Chi2/Rmax<5%). According to this model the c515H7 Mab Fc domain may be regarded as a mixture of two components. The major one representing 81% of the total amount shows a constant of dissociation between 380 and 450 nM, the minor one (19%) showed a constant of dissociation between 32 and 37 nM. According to the literature the heterogeneity observed with h-FcγRIIIA was probably linked to the glycosylation heterogeneity on the Mab Fc domain. The end of the association phase was close to reach the steady-state. A plot representing a mean of the response in RU (close to Req) at the end of the association phase versus the h-FcγRIIIA concentration (C) can be fitted with the mathematical model:

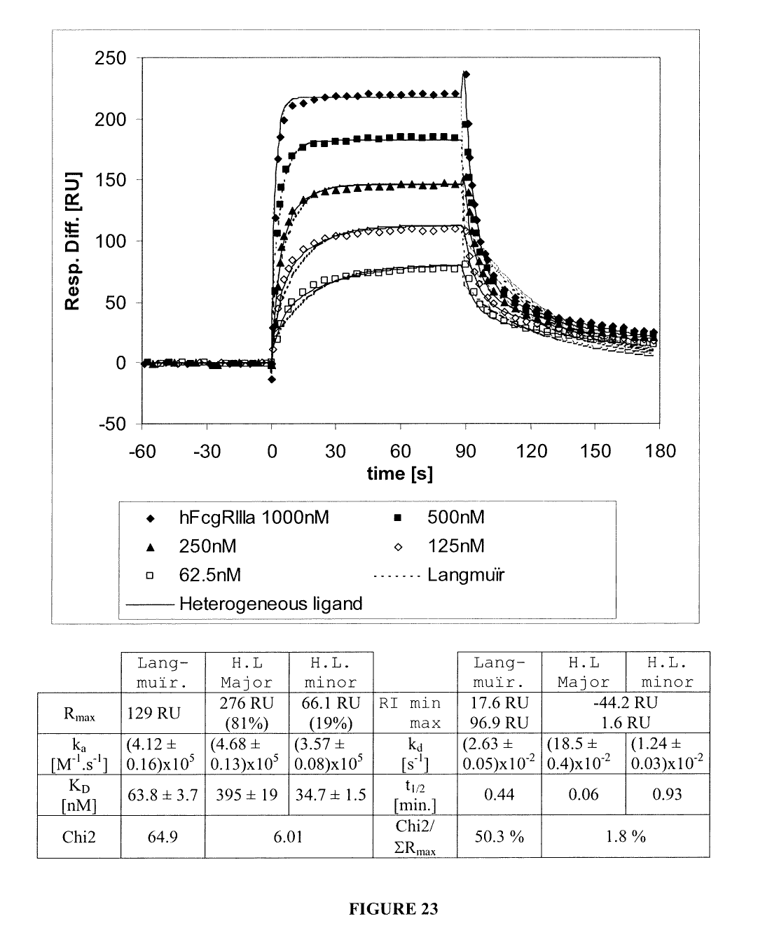

Req=(K_{A}·C·Rₘₐₓ)/K_{A}·C·n+1) with n=1 [FIG. 24]. The constant of dissociation K_{D} corresponding to 1/K_{A} was 160 nM.

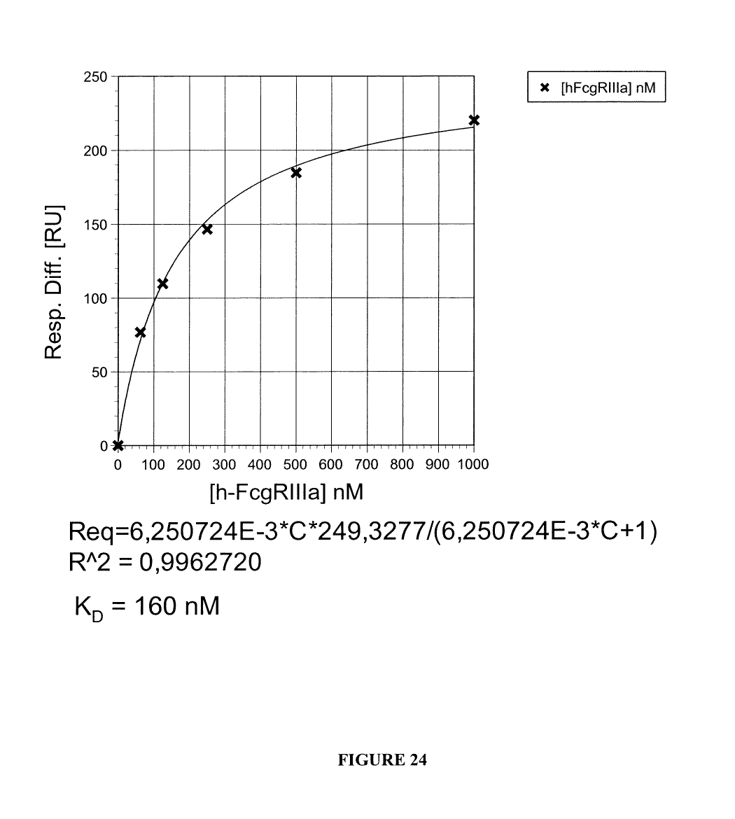

Sensorgrams obtained with m-FcγRI [FIG. 25] may be fitted by a Langmuïr model (5%<Chi2/Rmax<10%) but the “heterogeneous ligand” model improved significantly the quality of the fitting (Chi2/Rmax<2%). According to this model, the c515H7 Mab Fc domain may be regarded as a mixture of two components. The major one representing 81% of the total amount showed a constant of dissociation around 380 and 450 nM, the minor one (19%) showed a constant of dissociation between 32 and 37 nM.

The end of the association phase was close to reach the steady-state. A plot representing a mean of the response in RU (close to Req) at the end of the association phase versus the m-FcγRI concentration (C) can be fitted with the mathematical model:

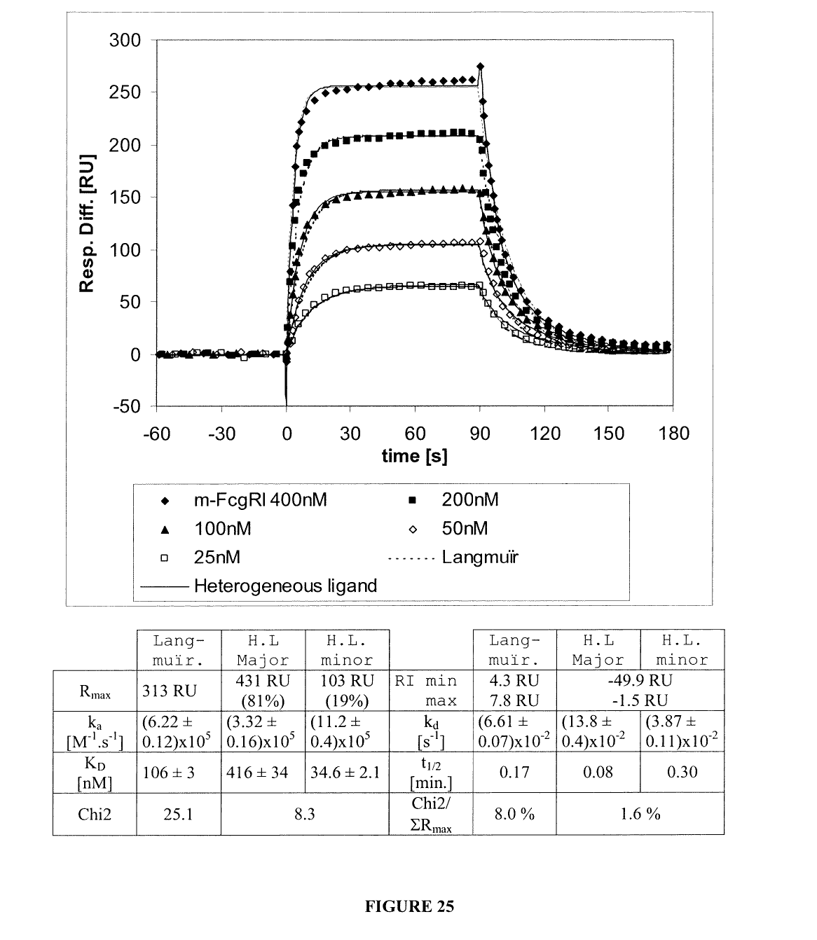

Req=(K_{A}·C·Rₘₐₓ)/K_{A}·C·n+1) with n=1 [FIG. 26]. The dissociation constant K_{D} corresponding to 1/K_{A} is the equal to 107 nM.

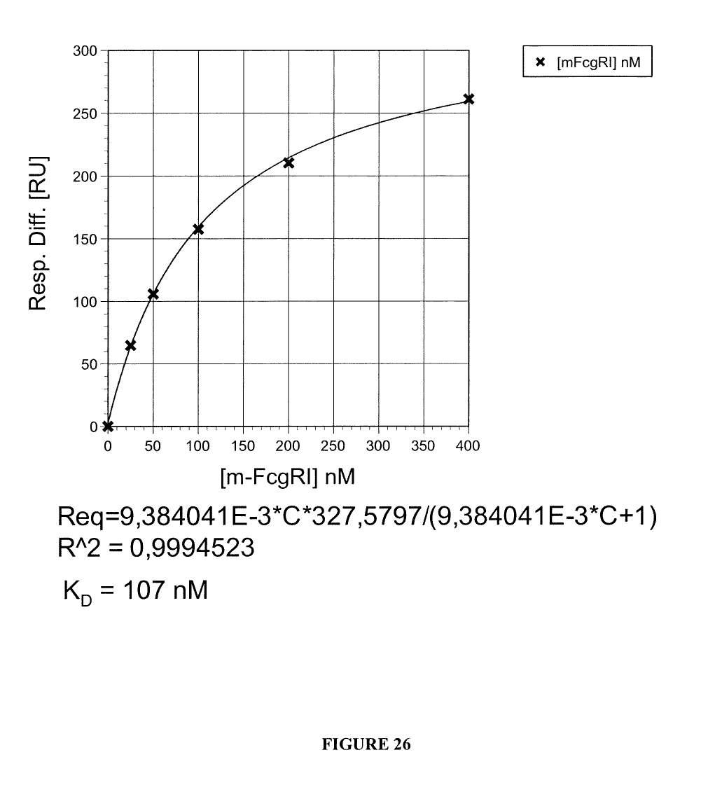

Sensorgrams obtained with m-FcγRIII [FIG. 27] were not perfectly fitted by a Langmuïr model (10%<Chi2/Rmax<20%) but the “heterogeneous ligand” model did not improve the quality of the fitting. Using the Langmuïr model the constant of dissociation was around 20 nM.

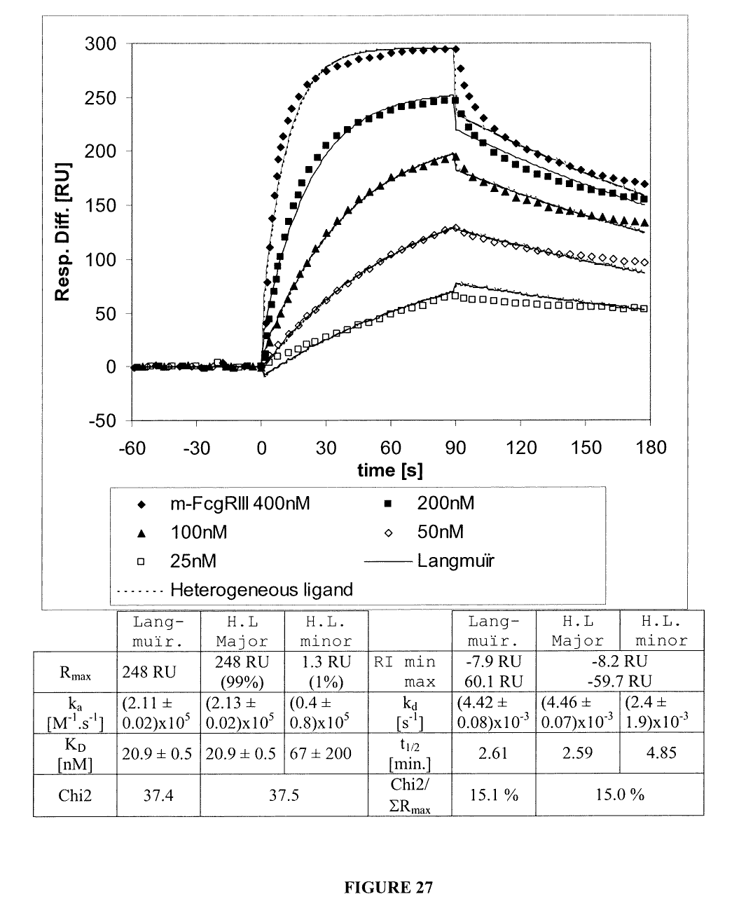

A ranking of the four Fc gamma receptors is presented in FIG. 28 representing Kd plot in function of the half-life of the complex. In accordance with the literature, h-FcγRI binds with high affinity and h-FcγRIIIA with a lower affinity to the Fc part of a human IgG1 isotype antibody. m-FcγRIII binds with an intermediate affinity between the affinity of the major component of c515H7 Mab for h-FcγRIIIA and the affinity for h-FcγRI. Both components of the c515H7 Mab interact with m-FcγRI in a similar way than with h-FcγRIIIA

### Example 12

### Antibody Dependant Cellular Cytotoxicity (ADCC) Effect of hz515H7VH1D76NVL2 (hz515H7) Mab on Cells Expressing CXCR4

ADCC was measured using the lactate dehydrogenase (LDH) release assay described above (see example 5).

Briefly, human PBMCs were isolated from volunteer healthy donors' blood using a Ficoll density gradient. NK cells were purified from the PBMCs fraction according to the Human NK Cell Enrichment Kit manufacturer's protocol. NK cells, used as effector cells (E), were mixed with RAMOS (lymphoma), DAUDI (lymphoma) or HeLa (cervix cancer) tumor target cells (T) at an E:T ratio of 50:1, said target cells having been previously pre-incubated for 10 minutes at room temperature with the hz515H7VH1D76NVL2 (hz515H7) antibody (10 μg/ml). After 4 hours incubation at 37° C., specific cell lysis was determined by measuring the amount of LDH released with the Cytotoxicity Detection Kit^{PLUS} according to the manufacturer's instructions.). Percent of cytotoxicity was calculated as follows: % lysis=[experimental release−effector and target spontaneous release]/[target maximum release−target spontaneous release]×100.

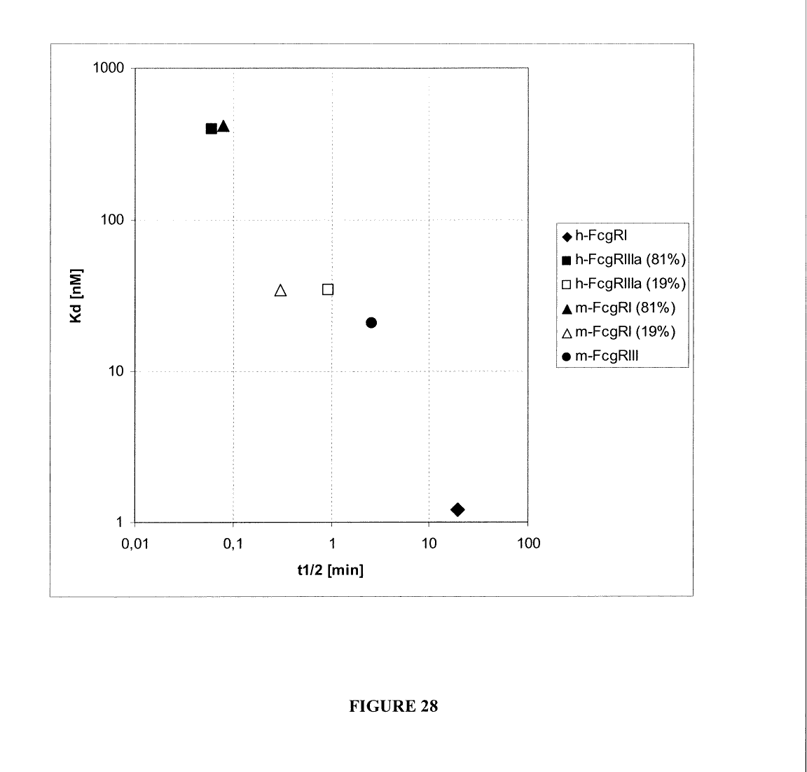

FIG. 29 shows ADCC on cells expressing CXCR4: RAMOS, DAUDI and HeLa cells. No effect was observed when cells were incubated with the hIgG1 isotype control (10 μg/ml). In contrast, hz515H7 Mab (10 μg/ml) was capable of inducing significant ADCC (around 40%) on RAMOS, DAUDI and HeLa cells.

### Example 13

### Complement Dependant Cytotoxicity (CDC) Effect of hz515H7VH1D76NVL2 (hz515H7) Mab on Cells Expressing CXCR4

CDC assay was based on ATP measurement using CellTiter Glo reagent (Promega, Madison, Wis., USA), as described in example 7.

Briefly, 10000 target cells were plated in 96-well flat bottom plates in presence of hz515H7VH1D76NVL2 (hz515H7) Mab. Following incubation at room temperature for 10 minutes, pooled human serum from healthy donors was added at a final concentration of 10%. After 1 h at 37° C., viability was determined by measuring the amount of ATP. Percent of cytotoxicity was calculated as follows: % Cytotoxicity=100−[[experimental/target cell without antibody]×100].

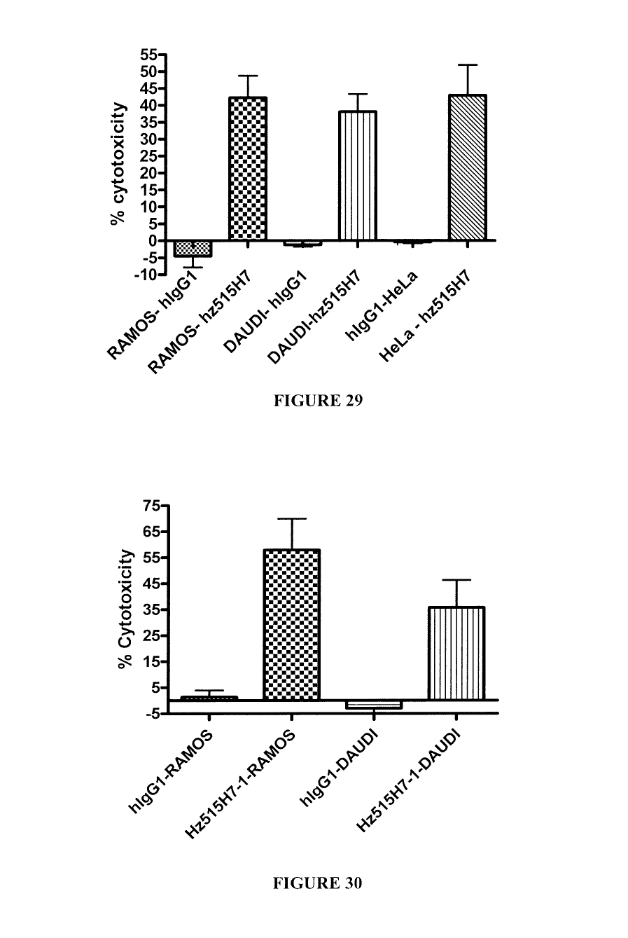

FIG. 30 shows CDC on cell lines expressing CXCR4: RAMOS and DAUDI cells. No effect was observed when cells were incubated with the hIgG1 isotype control (10 μg/mL). In contrast, hz515H7VH1D76NVL2 (hz515H7-1) Mab (10 μg/mL) was able to induce significant CDC: around 58% for RAMOS cells and 36% for DAUDI cells.

1. A humanized antibody binding to CXCR4, or a CH2-containing binding fragment thereof, said humanized antibody comprising a heavy chain variable domain selected from the sequences SEQ ID No. 7 to 10 and a light chain variable domain selected from the sequences SEQ ID No. 11 to 17; for use in a method of treatment of cancer by killing a CXCR4 expressing cancer cell by induction of at least one effector function, in the presence of effector cells or complement components.

2. A humanized antibody according to claim 1, characterized in that said effector function consists of the antibody-dependent cell cytotoxicity (ADCC).

3. A humanized antibody according to claim 1, characterized in that said effector function consists of the complement dependent cytotoxicity (CDC).

4. A humanized antibody according to claim 1, characterized in that said effector functions consist of the antibody-dependent cell cytotoxicity (ADCC) and the complement dependent cytotoxicity (CDC).

5. A humanized antibody according to any one of claims 1 to 4, wherein the said humanized antibody is selected in the group constituting of:
a humanized antibody comprising a heavy chain variable domain of sequence SEQ ID No. 8 and a light chain variable domain selected from the sequences SEQ ID No. 11 to 17;
a humanized antibody comprising a heavy chain variable domain selected from the sequences SEQ ID No. 7 to 10 and a light chain variable domain of sequence SEQ ID No. 13;
a humanized antibody comprising a heavy chain variable domain of sequence SEQ ID No. 8 and a light chain variable domain of sequence SEQ ID No. 13;
a humanized antibody comprising a heavy chain selected from the sequences SEQ ID No. 18 to 21 and/or a light chain selected from the sequences SEQ ID No. 22 to 28;
a humanized antibody comprising a heavy chain of sequence SEQ ID No. 19 and/or a light chain selected from the sequences SEQ ID No. 22 to 28;
a humanized antibody comprising a heavy chain selected from the sequences SEQ ID No. 18 to 21 and/or a light chain of sequence SEQ ID No. 24; and
a humanized antibody comprising a heavy chain of sequence SEQ ID No. 19 and/or a light chain of sequence SEQ ID No. 24.

6. A humanized antibody according to one of claims 1 to 7, characterized in that said antibody is an IgG1.

7. A humanized antibody according to one of the claims 1 to 6, characterized in that said CXCR4 expressing cancer cell consists of a malignant hematological cell.

8. A humanized antibody according to claim 7, characterized in that said CXCR4 malignant hematological cell is selected from the group comprising lymphoma cell, leukemia cell or multiple myeloma cell.

9. A humanized antibody according to one of claims 1 to 8, characterized in that said effector cells comprise NK cells, macrophages, monocytes, neutrophils or eosinophils.

10. A humanized antibody according to one of the claims 1 to 9, characterized in that it induces ADCC level on RAMOS lymphoma cells, after an incubation period of 4 hours, of at least 40%.

11. A humanized antibody according to one of the claims 1 to 10, characterized in that no significant ADCC is induced on NK cells.

12. A humanized antibody according to one of the claims 1 to 11, characterized in that said complement components comprise at least the C1q.

13. A humanized antibody according to one of the claims 1 to 12, characterized in that it induces CDC level on RAMOS lymphoma cells, after an incubation period of 1 hour, of at least 30%, preferentially of at least 50% and most preferably of at least 70%.

14. A humanized antibody according to one of the claims 1 to 13, characterized in that it induces CDC level on NIH3T3 CXCR4 cells, after an incubation period of 1 hour, of at least 30%, preferentially of at least 50% and most preferably of at least 70%.

15. A humanized antibody according to one of the claims 1 to 14, characterized in that the humanized antibody, or CH2-containing binding fragment thereof binds at least one human FcγRs.

16. A humanized antibody according to the claim 15, characterized in that said at least one FcγRs is human FcγRI.

17. A humanized antibody according to the claim 16, characterized in that it binds said FcγRI with a constant of dissociation (KD), according to the Langmuïr model, between 1 and 10 nM.

18. A humanized antibody according to the claim 17, characterized in that said at least one FcγRs is human FcγRIIIA.

19. A humanized antibody according to the claim 18, characterized in that it binds said FcγRIIIA with a constant of dissociation (KD), according to the heterogeneous ligand model, between 200 and 1000 nM.

20. A humanized antibody binding to CXCR4, or a CH2-containing binding fragment thereof, for use in a method of treatment of cancer by killing CXCR4 expressing cancer cells; said human or humanized antibody comprising a heavy chain variable domain selected from the sequences SEQ ID No. 7 to 10 and a light chain variable domain selected from the sequences SEQ ID No. 11 to 17; wherein at least one effector function of the said human or humanized antibody is induced, in the presence of effector cells or complement components.

21. A humanized antibody according to claim 20, characterized in that said cancer consists of lymphoma.

22. A method for the screening of humanized antibodies binding to CXCR4, or CH2-containing binding fragments thereof, for use in killing a CXCR4 expressing cancer cell by induction of at least one effector function, in the presence of effector cells or complement components, wherein said method comprises at least one selection step selected from:
selecting antibodies inducing an ADCC level on RAMOS lymphoma cells, after an incubation period of 4 hours, of at least 40%;
selecting antibodies inducing a CDC level on RAMOS lymphoma cells, after an incubation period of 1 hour, of at least 30%, preferentially of at least 50% and most preferably of at least 70%;
selecting antibodies inducing a CDC level on NIH3T3 CXCR4 cells, after an incubation period of 1 hour, of at least 30%, preferentially of at least 50% and most preferably of at least 70%;
selecting antibodies binding FcγRI with a constant of dissociation (KD), according to the Langmuïr model, between 1 and 10 nM;
selecting antibodies binding FcγRIIIA with a constant of dissociation (KD), according to the heterogeneous ligand model, between 200 and 1000 nM.
